(19) United States
(12) Patent Application Publication
(10) Pub. No.: US 2024/0100072 A1
(43) Pub. Date: Mar. 28, 2024

(54) SUBCUTANEOUSLY ADMINISTERED TREATMENTS FOR ADVANCED PARKINSON'S DISEASE

(71) Applicant: AbbVie Inc., North Chicago, IL (US)

(72) Inventors: Maurizio F. Facheris, Chicago, IL (US); Michael Gold, Deerfield, IL (US); Weining Z. Robieson, Hawthorn Woods, IL (US); Melissa Vos, Chicago, IL (US); Amy M. Spiegel, Grayslake, IL (US); Nahome Tezera Fisseha, Arlington, VA (US); Janet A. Benesh, Kennesaw, GA (US); George Liossis, Downers Grove, IL (US); Kumar Budur, Chicago, IL (US)

(21) Appl. No.: 17/975,267

(22) Filed: Oct. 27, 2022

Related U.S. Application Data
(60) Provisional application No. 63/327,441, filed on Apr. 5, 2022.
(60) Provisional application No. 63/318,567, filed on Mar. 10, 2022.
(60) Provisional application No. 63/297,513, filed on Jan. 7, 2022.
(60) Provisional application No. 63/291,207, filed on Dec. 17, 2021.
(60) Provisional application No. 63/272,574, filed on Oct. 27, 2021.

(30) Foreign Application Priority Data
Apr. 25, 2022 (CA) 3156401

(51) Int. Cl. A61K 31/661; A61K 9/00; A61P 25/16

(52) U.S. Cl. CPC A61K 31/661; A61K 9/0019; A61P 25/16

(57) ABSTRACT

The present disclosure relates to safe and effective subcutaneously administered treatments for advanced Parkinson's disease.

### CROSS-REFERENCE TO RELATED APPLICATIONS

This application claims the benefit of priority to U.S. Provisional Application No. 63/272,574, filed Oct. 27, 2021; U.S. Provisional Application No. 63/291,207, filed Dec. 17, 2021; U.S. Provisional Application No. 63/297,513, filed Jan. 7, 2022; U.S. Provisional Application No. 63/318,567, filed Mar. 10, 2022; U.S. Provisional Application No. 63/327,441, filed Apr. 5, 2022; and Canadian Application No. 3,156,401, filed Apr. 25, 2022. The entire content of each of these applications is incorporated herein by reference in its entirety.

### FIELD OF THE INVENTION

The present disclosure relates to safe, tolerable, and effective subcutaneously administered treatments for advanced Parkinson's disease.

### BACKGROUND

Parkinson's disease is a chronic and progressive neurodegenerative condition characterized by reduced levels in the brain of the neurotransmitter dopamine (i.e., 3,4-dihydroxyphenethylamine). Administration of levodopa (i.e., L-3,4-dihydroxyphenylalanine) currently is the most effective therapy for treating Parkinson's disease patients. Co-administration of carbidopa with levodopa inhibits the peripheral metabolism of levodopa to dopamine, which significantly reduces the levodopa dose required for a therapeutically effective clinical response and reduces the associated side effects.

To date, there remains a need for safe, tolerable, and effective subcutaneously administered treatments for the treatment of advanced Parkinson's disease.

### SUMMARY OF THE INVENTION

The present disclosure relates to safe, tolerable, and effective subcutaneously administered therapies for the treatment of advanced Parkinson's disease.

Stable liquid formulations comprising a combination of a levodopa phosphate prodrug “foslevodopa” and a carbidopa phosphate prodrug “foscarbidopa,” which are suitable for use in the present disclosure are described in International Publication No. WO 2020/102628, which is hereby expressly incorporated by reference in its entirety.

As disclosed herein, the present disclosure relates to the following embodiments.

Embodiment 1. A method of treating advanced Parkinson's disease in a subject by subcutaneous administration of a composition comprising foslevodopa and foscarbidopa, wherein the composition achieves an increase in “On” time without troublesome dyskinesia.

Embodiment 2. A method of treating advanced Parkinson's disease by subcutaneous administration of a composition comprising foslevodopa and foscarbidopa, wherein the composition achieves a decrease in “Off” time without troublesome dyskinesia.

Embodiment 3. A method of treating advanced Parkinson's disease by subcutaneous administration of a composition comprising foslevodopa and foscarbidopa, wherein the composition achieves a decrease in sleep symptoms as assessed by the Parkinson's Disease Sleep Scale-2 (PDSS-2) total score.

Embodiment 4. A method of treating advanced Parkinson's disease by subcutaneous administration of a composition comprising foslevodopa and foscarbidopa, wherein the composition achieves an increase in PD-related quality of life as assessed by the Parkinson's Disease Questionnaire-39 item (PDQ-39) summary index.

Embodiment 5. A method of treating advanced Parkinson's disease by subcutaneous administration of a composition comprising foslevodopa and foscarbidopa, wherein the composition achieves an increase from baseline to final visit in health-related quality of life as assessed by the EQ-5D-5L summary index.

Embodiment 6. A method of treating advanced Parkinson's disease by subcutaneous administration of a composition comprising foslevodopa and foscarbidopa wherein the composition achieves a decrease from baseline to final visit in PD symptoms as assessed by the wearable motion sensor device.

Embodiment 7. The method of any one of embodiments 1-6, wherein the composition is administered in an amount effective for reducing parkinsonian symptoms by at least 46% from baseline.

Embodiment 8. The method of any one of embodiments 1-6, wherein the composition is administered in an amount effective for reducing the baseline MDS-UPDRS total score by at least 9 units.

Embodiment 9. The method of any one of embodiments 1-6, wherein the composition is administered in an amount effective for reducing the baseline PDQ-39 score by at least 6.9 units.

Embodiment 10. The method of any one of embodiments 1-6, wherein the composition is administered in an amount effective for reducing the baseline PDSS-2 total score by at least 2 units.

Embodiment 11. A method of any one of embodiments 1-6, wherein the treatment duration is at least 10 days with no incidence of developing skin nodules.

Embodiment 12. A method of reducing incidences of “Off” time of parkinsonian symptoms in a subject compared with the subject receiving oral administration of tablets containing levodopa and carbidopa, according to any one of embodiments 1 to 6.

Embodiment 13. A method of embodiment 12, wherein the incidences of “Off” time of parkinsonian symptoms in a subject are reduced while increasing “On” time without troublesome dyskinesia.

Embodiment 14. A method of improving average daily normalized “Off” time as assessed by the PD diary in a subject compared with the subject receiving oral administration of tablets containing levodopa and carbidopa, according to any one of embodiments 1-6.

Embodiment 15. A method of improving Motor Aspects of Experiences of Daily Living as assessed by the MDS-UPDRS Part II score in a subject compared with the subject receiving oral administration of tablets containing levodopa and carbidopa, according to any one of embodiments 1-6.

Embodiment 16. A method of reducing morning akinesia in a subject compared with the subject receiving oral administration of tablets containing levodopa and carbidopa, according to any one of embodiments 1-6.

Embodiment 17. A method of improving health-related quality of life as assessed by the EQ-5D-5L summary index in a subject compared with the subject receiving oral administration of tablets containing levodopa and carbidopa, according to any one of embodiments 1-6.

Embodiment 18. A method of improving median bradykinesia score (BK50) as assessed by the wearable motion sensor device in a subject compared with the subject receiving oral administration of tablets containing levodopa and carbidopa, according to any one of embodiments 1-6.

Embodiment 19. A method of improving interquartile range of bradykinesia score (BK75-BK25) as assessed by the wearable motion sensor device in a subject compared with the subject receiving oral administration of tablets containing levodopa and carbidopa, according to any one of embodiments 1-6.

Embodiment 20. A method of improving median dyskinesia score (DK50) as assessed by the PKG wearable device in a subject compared with the subject receiving oral administration of tablets containing levodopa and carbidopa, according to any one of embodiments 1-6.

Embodiment 21. A method of improving of tremor and daytime somnolence as assessed by the wearable motion sensor device in a subject compared with the subject receiving oral administration of tablets containing levodopa and carbidopa, according to any one of embodiments 1-6.

Embodiment 22. A method of improving MDS-UPDRS Part I score, Part III score, Part IV score, and total score of Parts I-III in a subject compared with the subject receiving oral administration of tablets containing levodopa and carbidopa, according to any one of embodiments 1-6.

Embodiment 23. A method of improving “On” time with non-troublesome dyskinesia, and “On” time with troublesome dyskinesia as assessed by a PD Diary in a subject compared with the subject receiving oral administration of tablets containing levodopa and carbidopa, according to any one of embodiments 1-6 to a patient in need of treatment for Parkinson's disease.

Embodiment 24. A method of improving average daily normalized “Off” time, “On” time without troublesome dyskinesia, “On” time without dyskinesia, “On” time with non-troublesome dyskinesia, and “On” time with troublesome dyskinesia as assessed by the PD Diary in a subject compared with the subject receiving oral administration of tablets containing levodopa and carbidopa, according to any one of embodiments 1-6 to a patient in need of treatment for Parkinson's disease.

Embodiment 25. A method of improving average daily absolute “Off” time, “On” time without troublesome dyskinesia, “On” time without dyskinesia, “On” time with non-troublesome dyskinesia, “On” time with troublesome dyskinesia, and “Asleep” time as assessed by the PD Diary without normalizing in a subject compared with the subject receiving oral administration of tablets containing levodopa and carbidopa, according to any one of embodiments 1-6.

Embodiment 26. A method of improving PDSS-2 domain scores in a subject compared with the subject receiving oral administration of tablets containing levodopa and carbidopa, according to any one of embodiments 1-6.

Embodiment 27. A method of improving PDQ-39 domain scores in a subject compared with the subject receiving oral administration of tablets containing levodopa and carbidopa, according to any one of embodiments 1-6.

Embodiment 28. A method of improving EQ-5D-5L visual analogue scale (VAS) score in a subject compared with the subject receiving oral administration of tablets containing levodopa and carbidopa, according to any one of embodiments 1-6.

Embodiment 29. The method of any one of embodiments 1-28, wherein the weight ratio of foslevodopa to foscarbidopa is about 20:1.

Embodiment 30. The method of any one of embodiments 1-29, wherein the composition comprises about 240 mg/mL of foslevodopa and about 12 mg/mL of foscarbidopa.

Embodiment 31. The method of any one of embodiments 1-30, wherein the composition has a pH of between about 6.6 and about 8.1.

Embodiment 32. The method of any one of embodiments 1-6, wherein the composition has an osmolality up to about 2700 mOsmol/kg.

Embodiment 33. The method of any one of embodiments 1-6, wherein the composition has an osmolality between about 2200 and about 2500 mOsmol/kg.

Embodiment 34. A method of improving “On” time without troublesome dyskinesia as assessed by a PD Diary in a subject compared with the subject receiving oral administration of tablets containing levodopa and carbidopa, according to any one of embodiments 1-6 to a patient in need of treatment for Parkinson's disease, wherein the average time to reach “On” time is between 10-60 minutes.

Embodiment 35. The method of embodiment 34, wherein the average time to reach “On” time is from about 15 minutes to about 50 minutes.

Embodiment 35A The method of embodiment 35, wherein the average time to reach “On” time is from about 20 minutes to about 40 minutes, or about 30 minutes.

Embodiment 35B. The method of embodiment 35A, wherein the time to reach “On” time is 28.9 minutes.

Embodiment 36. A method of improving “On” time with non-troublesome dyskinesia as assessed by a PD Diary in a subject compared with the subject receiving oral administration of tablets containing levodopa and carbidopa, according to any one of embodiments 1-6 to a patient in need of treatment for Parkinson's disease, wherein the frequency of “On” time is about twice as frequent relative to the subject receiving oral administration of tablets containing levodopa and carbidopa.

Embodiment 37. A method of improving “Off” time with non-troublesome dyskinesia as assessed by a PD Diary in a subject compared with the subject receiving oral administration of tablets containing levodopa and carbidopa, according to any one of embodiments 1-6 to a patient in need of treatment for Parkinson's disease, wherein the frequency of “Off” time within 30 minutes of waking is about one-fourth less frequent relative to the subject receiving oral administration of tablets containing levodopa and carbidopa.

Embodiment 38. The method of any one of embodiments 1-6, wherein the frequency of “Off” time is sustained for at least 24 hours.

Further benefits of the present disclosure will be apparent to one skilled in the art from reading this patent application. The embodiments of the disclosure described in the following paragraphs are intended to illustrate the invention and should not be deemed to narrow the scope of the invention.

### BRIEF DESCRIPTION OF THE DRAWINGS

FIG. 1 is a study design schematic.

FIG. 2 is a graphical representation showing distribution of Modal Daily Dose for Oral CD/LD Group.

FIG. 3 is a graphical representation showing distribution of Modal Daily Dose for foslevodopa and foscarbidopa Group.

FIG. 4 is a graphical representation showing LS Mean Change from Baseline (±SE) in Average Daily Normalized “On” Time without Troublesome Dyskinesia by Treatment Group Over the DB Treatment Period (Full Analysis Set).

FIG. 5 is a graphical representation showing S Mean Change from Baseline (±SE) in Average Daily Normalized “Off” Time by Treatment Group Over the DB Treatment Period (Full Analysis Set).

FIG. 6 is a graphical representation showing LS Mean Change from Baseline (±SE) in MDS-UPDRS Part II Score by Treatment Group Over the DB Treatment Period (Full Analysis Set).

FIG. 7 is a graphical representation showing Distribution of Average Daily Normalized “Off” and “On” times Over Time (Full Analysis Set).

FIG. 8 is a graphical representation showing Distribution of Early Morning Non-Sleep Symptoms Over Time (Full Analysis Set).

FIG. 9 shows results of Study Blinding Assessment: Safety Analysis Set.

FIG. 10 is a graphical representation showing normalized “Off” and “On” times: number of hours and as a percent of waking day.

FIG. 11 shows an exemplary Parkinson's disease diary.

FIG. 12 is a graphical representation of the percentage of subjects by the highest grade received on any of the 10 Days for the pharmaceutical composition (levodopa 4′-monophosphate and carbidopa 4′-monophosphate at a 20:1 ratio) and placebo on the Infusion Site Evaluation Numeric Scale.

FIG. 13 is a graphical representation of the percentage of subjects by the highest grade received on any of the 10 Days for a pharmaceutical composition of levodopa 4′-monophosphate and carbidopa 4′-monophosphate at a 20:1 ratio and placebo on the Infusion Site Evaluation Letter Grade Scale.

FIG. 14A a graphical representation of the study design for Clinical Study A to evaluate the safety, efficacy and tolerability of a 12-week continuous subcutaneous infusion of a pharmaceutical composition of levodopa 4′-monophosphate and carbidopa 4′-monophosphate at a 20:1 ratio in patients.

FIG. 14B is a graph showing the results for Clinical Study A of the infusion site grading using the numeric scale in patients.

FIG. 14C is a graph showing the results for Clinical Study A of the infusion site grading using the letter scale in patients.

FIG. 14D is a graph showing Mean (SD) Change from Baseline in MDS-UPDRS Total Scores for Clinical Study A in patients.

FIG. 14E is a graph showing Mean (SD) Change from Baseline in “Off” time at Regularly Scheduled Visits for Clinical Study A in patients.

FIG. 15 is a plasma time-concentration profile of levodopa plasma levels (±standard deviation) in healthy human volunteers after subcutaneous administration of a bolus dose of a pharmaceutical composition of levodopa 4′-monophosphate and carbidopa 4′-monophosphate at a ratio of 20:1 followed by a continuous subcutaneous dose of the pharmaceutical composition for 24 hours (Clinical Study B).

FIG. 16 shows the percentage of patients experiencing “Off” time during a day receiving the pharmaceutical composition compared with patients receiving oral Sinemet© (Clinical Study B).

FIG. 17A shows Mean Levodopa Exposure (±SD) following 24-hour foslevodopa and foscarbidopa Infusion and 16-hour Duopa infusion followed by Oral LD/CD Doses at 18 and 21 hours.

FIG. 17B shows Mean Levodopa Exposure (±SD) following 24-hour foslevodopa and foscarbidopa Infusion and 24 hour Duopa infusion.

FIG. 18 shows a Study Design Schematic. D=Day; V=Visit; W=Week.

FIG. 19 shows an illustration of Narrowing of Levodopa Therapeutic Window as Parkinson's disease (PD) Progresses

FIG. 20 shows a schematic of the Study Design.

FIG. 21 shows observed mean over time in average daily normalized “Off” time, “On” time with troublesome dyskinesia, and “On” time without troublesome dyskinesia.

FIG. 22 shows average daily normalized times from PD diary, mean hours and as percentage of waking day.

FIG. 23 shows modal total daily dose (mg LD by molecular weight)

FIG. 24 shows change from baseline to 6 Months in efficacy outcomes. * P<0.05; **P<0.01. MDS-UPDRS II, Movement Disorder Society Unified Parkinson's Disease Rating Scale part II; PD, Parkinson's disease; PDQ-39, 39-item Parkinson's disease Questionnaire.

FIG. 25 shows adjusted percentage of responders (≥1-hour) and robust responders (≥3-hour) for “Off”-time and “On”-time without troublesome dyskinesia (ONwoTD) between foslevodopa and foscarbidopa vs levodopa and carbidopa immediate release oral tablets (LCIR) at week 12.

FIG. 26 shows adjusted percentage of responders (≥1-hour) and robust responders (≥3-hour) for “Off”-time and “On”-time without troublesome dyskinesia (ONwoTD) for foslevodopa and foscarbidopa at week 13 and 52.

FIG. 27 shows change in “On”-time without troublesome dyskinesia (“Good On”) from baseline to week 12 for foslevodopa and foscarbidopa vs levodopa and carbidopa immediate release oral tablets (LCIR).

FIG. 28 shows change in “Off”-time from baseline to week 12 for foslevodopa and foscarbidopa vs levodopa and carbidopa immediate release oral tablets (LCIR).

FIG. 29 shows Sankey diagrams of motor-state transition categories for baseline and week 12 visits for (a) levodopa and carbidopa immediate release oral tablets (LCIR) and (b) foslevodopa and foscarbidopa patients.

FIG. 30 shows Proportion of Patients and Number or Parkinson's Disease Medication Administered in Example 14.

### DETAILED DESCRIPTION OF THE INVENTION

The present disclosure describes the unexpected discovery that subcutaneous administration of a pharmaceutical composition, foscarbidopa/foslevodopa, at 12 mg/240 mg per ml solution for subcutaneous infusion, to advanced Parkinson's disease patients who are not adequately controlled with current medical therapy, is safe, tolerable, and effective for the treatment of motor fluctuations.

As used herein, the terms “levodopa phosphate prodrug,” “levodopa-4′-monophosphate,” and “foslevodopa” are used interchangeably to refer to the compound:

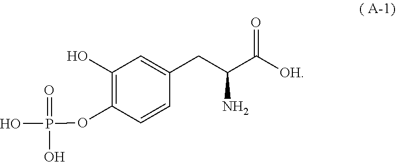 [SMILES: N[C@@H](Cc1ccc(OP(=O)(O)O)c(O)c1)C(=O)O]

which has been assigned the CAS registry number 97321-87-4.

As used herein, the terms “carbidopa phosphate prodrug,” “carbidopa-4′-monophosphate,” and “foscarbidopa” are used interchangeably to refer to the compound:

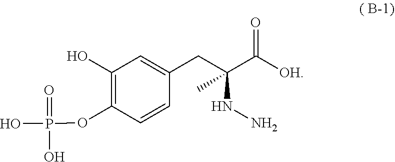 [SMILES: C[C@@](Cc1ccc(OP(=O)(O)O)c(O)c1)(NN)C(=O)O]

which has been assigned the CAS registry number 28860-95-9. As used herein, the terms “foscarbidopa/foslevodopa for subcutaneous infusion,” “foscarbidopa/foslevodopa,” and “the product of the present disclosure” may be used interchangeably.

Where a numeric range is recited herein, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated.

The singular forms “a,” “an” and “the” include plural referents unless the context clearly dictates otherwise.

The term “and/or” as used in a phrase such as “A and/or B” herein is intended to mean “A and B”, “A or B”, “A” or “B”.

The term “about” generally refers to a range of numbers that one of skill in the art would consider equivalent to the recited value (i.e., having the same function or result). In many instances, the term “about” may include numbers that are rounded to the nearest significant figure. In certain instances, the term “about” may be used to denote values falling within ±20% of the recited values, e.g. within ±15%, ±10%, ±7.5%, ±5%, ±4%, ±3%, ±2% or ±1% of the recited values.

The term “baseline” means the first measurement of the targeted variable just before the administration of the studied therapy.

The term “container” means any coated or uncoated suitable container made using any suitable material, and includes, but is not limited to vials, cartridges, syringes, bottles, and materials such as glass, plastic, and/or any combinations thereof.

Unless the context requires otherwise, the terms “comprise,” “comprises,” and “comprising” are used on the basis and clear understanding that they are to be interpreted inclusively, rather than exclusively, such that they indicate the inclusion of the recited feature but without excluding one or more other such features.

The term “patient”, “subject”, “individual” and the like refers to humans.

The term “carrier” used in connection with a pharmaceutical excipient refers to any and all solvents, dispersion media, preservatives, coatings, isotonic and absorption delaying agents, and the like, that are compatible with pharmaceutical administration.

As used herein, the term ““Off” time” refers to periods of the day when medications are not working well, causing reappearance or worsening of parkinsonian symptoms (including, for example, tremor, rigidity, bradykinesia, as well as non-motor symptoms such as depression, pain, anxiety).

As used herein, the term ““On” time” refers to periods of adequate control of symptoms.

As used herein, the term “dyskinesia” refers to neurological syndromes, natural and drug induced, consisting of spontaneous and largely uncontrollable movements or paucity of movement. Subjects, when evaluating their dyskinesia determine whether it is “troublesome” (e.g., involuntary movements) or “not troublesome.”

As described herein, the pharmaceutical composition can have a final pH of about 5 to 11, including pH values increasing in increments of 0.1 in between 5 to 11. In certain embodiments that pH may be between 6.5 to about pH 9.2, including pH values increasing in increments of 0.1 in between 6.5 and 9.2. In one embodiment, the pharmaceutical composition has a final pH (e.g. after reconstitution with water) of between about 6.8 and about 7.8. Thus, the pharmaceutical composition may have a final pH selected from about 6.8, 6.9, 7.0, 7.1, 7.2, 7.3, 7.4, 7.5, 7.6, 7.7, or 7.8. In one embodiment, the pharmaceutical composition has final pH of between about 7.0 and about 7.5. Thus, the pharmaceutical composition may have a final pH selected from about 7.0, 7.1, 7.2, 7.3, 7.4, or 7.5.

This disclosure also relates to therapeutic methods in which the present pharmaceutical compositions are used. In one aspect, the disclosure provides a method of treating advanced Parkinson's disease in a subject, comprising subcutaneously administering a pharmaceutical composition as disclosed herein to a patient in need thereof. In some aspects and embodiments, the disclosure provides methods in which specific measures of advanced Parkinson's disease are improved. Thus, the disclosure provides a method of improving “Off” time of parkinsonian symptoms in a subject, the method comprising subcutaneously administering a pharmaceutical composition as disclosed herein to a patient in need of treatment for advanced Parkinson's disease in an amount effective for reducing parkinsonian symptoms by at least 46% from baseline.

The disclosure also provides a method of treating Parkinson's disease in a subject, comprising subcutaneously administering a pharmaceutical composition as disclosed herein to a subject in need of treatment for Parkinson's disease with a baseline Movement Disorder Society-Unified Parkinson's disease Rating Scale (MDS-UPDRS) total score in an amount effective for reducing the baseline MDS-UPDRS total score by about 5 units to about 15 units. The disclosure also provides a method of treating Parkinson's disease in a subject, comprising subcutaneously administering a pharmaceutical composition as disclosed herein to a subject in need of treatment for Parkinson's disease with a baseline Movement Disorder Society-Unified Parkinson's disease Rating Scale (MDS-UPDRS) total score in an amount effective for reducing the baseline MDS-UPDRS total score by at least 9 units. The disclosure also provides a method of treating Parkinson's disease in a subject, comprising subcutaneously administering a pharmaceutical composition as disclosed herein to a subject in need of treatment for Parkinson's disease with a baseline Parkinson's disease Questionnaire-39 items (PDQ-39) summary index score in an amount effective for reducing the baseline PDQ-39 score by about 2 units to about 10 units. The disclosure also provides a method of treating Parkinson's disease in a subject, comprising subcutaneously administering a pharmaceutical composition as disclosed herein to a subject in need of treatment for Parkinson's disease with a baseline Parkinson's disease Questionnaire-39 items (PDQ-39) summary index score in an amount effective for reducing the baseline PDQ-39 score by at least 6.9 units.

The disclosure also provides a method of treating Parkinson's disease in a subject, comprising subcutaneously administering a pharmaceutical composition as disclosed herein to a subject in need of treatment for Parkinson's disease with a baseline Parkinson's disease Sleep Scale-2 (PDSS-2) total score in an amount effective for reducing the baseline PDSS-2 total score by about 1 units to about 10 units. The disclosure also provides a method of treating Parkinson's disease in a subject, comprising subcutaneously administering a pharmaceutical composition as disclosed herein to a subject in need of treatment for Parkinson's disease with a baseline Parkinson's disease Sleep Scale-2 (PDSS-2) total score in an amount effective for reducing the baseline PDSS-2 total score by at least 2 units. In certain embodiments, the treatments disclosed herein can be continued for at least 10 days with no incidence of the subject developing skin nodules. The disclosure also provides a method of reducing incidences of “Off” time of parkinsonian symptoms in a subject compared with the subject receiving oral administration of tablets containing levodopa and carbidopa, comprising subcutaneously administering a pharmaceutical composition as disclosed herein to a patient in need of treatment for advanced Parkinson's disease.

### EXAMPLES

### Example 1: A Randomized, Double-Blind, Double-Dummy, Active-Controlled Study Comparing the Efficacy, Safety and Tolerability of foscarbidopa/foslevodopa for subcutaneous infusion to Oral CD/LD in Advanced Parkinson's Disease (aPD) Patients (Phase 3)

### Clinical Study A

### Study Design and Objectives

Clinical Hypothesis: The 24-hour/day CSCI of foscarbidopa/foslevodopa for subcutaneous infusion will increase “On” time without troublesome dyskinesia (“On” time without dyskinesia plus “On” time with non-troublesome dyskinesia), reduce “Off” time, and improve the Motor Aspects of Experiences of Daily Living compared to CD/LD immediate release (IR) tablets in patients with aPD whose motor fluctuations are inadequately controlled by their current Parkinson's Disease PD medications.

The study was a Phase 3, randomized, double-blind, double-dummy, 12-week, parallel group, active-controlled, multicenter study assessing the efficacy, safety, and tolerability of 24-hour/day continuous subcutaneous infusion (CSCI) of foscarbidopa/foslevodopa for subcutaneous infusion in the treatment of subjects with aPD whose motor fluctuations are inadequately controlled by their current medications.

Primary objective: Demonstrate the superiority of continuous subcutaneous infusion (CSCI) of foscarbidopa/foslevodopa for subcutaneous infusion over oral carbidopa/levodopa (CD/LD) immediate release (IR) tablets for the treatment of motor fluctuations in subjects with advanced Parkinson's disease (aPD) after 12 weeks of therapy.

Secondary objective: Assess the local and systemic safety and tolerability of foscarbidopa/foslevodopa for subcutaneous infusion subcutaneous infusion delivered as a CSCI for 24 hours daily for 12 weeks.

Study Design:

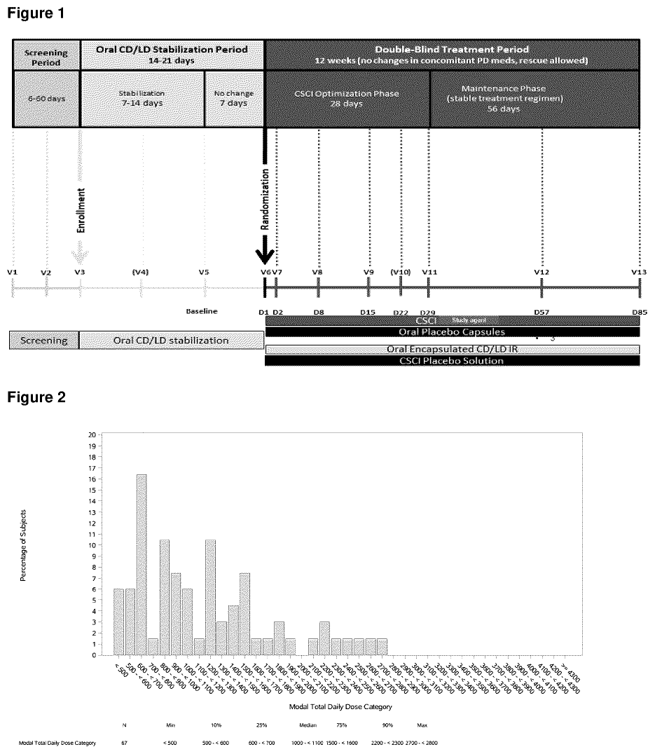

The schematic of the study is shown in FIG. 1, where CD/LD=carbidopa/levodopa; CSCI=continuous subcutaneous infusion; D=Day; IR=Immediate Release; PD=Parkinson's Disease; V=Visit.
a. V3 can occur at least 6 days after V1 to allow for V1 results to be fully available for review and for adequate infusion delivery system training and familiarity.
b. Screening Period activities will last at least 6 days; PD Diaries will be collected for at least 3 consecutive days during the Screening Period prior to V3; collection of PD Diaries may be repeated only once for 3 additional consecutive days leading up to V3 prior to the start of the Oral CD/LD Stabilization Period.
c. The activities of the Oral CD/LD Stabilization Period are expected to take 14 to 21 days to accommodate scheduling, additional unscheduled visits, and, when allowed, repeated assessments. PD Diaries will be collected for at least 3 consecutive days before Day −1.
d. If a subject prematurely discontinues study participation or completes the study and does not enter the open-label extension Study M20-098, a follow-up visit or follow-up phone call will be completed 30 days after the last day of use of the product of the disclosure (foscarbidopa/foslevodopa for subcutaneous infusion), if the subject is willing, to ensure all treatment-emergent AEs/SAEs have resolved.

The primary objective was to demonstrate the superiority of CSCI of foscarbidopa/foslevodopa for subcutaneous infusion over oral CD/LD IR tablets for the treatment of motor fluctuations in subjects with aPD after 12 weeks of therapy.

The secondary objective was to assess the local and systemic safety and tolerability of the product of the present disclosure (foscarbidopa/foslevodopa for subcutaneous infusion) delivered as a CSCI for 24 hours daily for 12 weeks.

### Screening Period

This 6- to 60-day period consists of 2 screening visits (V1 and V2).

### Oral CD/LD Stabilization Period

This 14-to 21-day period consists of 3 visits (V3, V4, and V5). V4 is an optional visit. V5 is the Baseline Visit for the majority of assessments.

### Double-Blind Treatment Period

This 12-week period starts with initiation of blinded foscarbidopa/foslevodopa for subcutaneous infusion and blinded oral capsule and consists of 2 parts: a 4-week CSCI Optimization Phase and an 8-week Maintenance Phase.

### Post-Treatment Period Activities

There are no post-treatment activities except for subjects who prematurely discontinue study participation.

### Treatment Assignment and Blinding

At the end of the Oral CD/LD Stabilization Period, eligible subjects were randomized in a 1:1 ratio to one of the two treatment arms for the 12-week Double-Blind Treatment Period:
24-hour/day CSCI of the product of the present disclosure (foscarbidopa/foslevodopa for subcutaneous infusion) plus oral placebo capsules for CD/LD IR (also referred to as investigational group or foscarbidopa/foslevodopa for subcutaneous infusion group)
OR
24-hour/day CSCI of a placebo solution for the product of the present disclosure plus oral encapsulated CD/LD IR tablets (also referred to as active control group or Oral CD/LD group)

Randomization was stratified by study site with a block size of 2.

The first day of the blinded administration of foscarbidopa/foslevodopa for subcutaneous infusion is defined as Day 1.

To help evaluate maintenance of the blind, an exit interview was conducted when subjects prematurely discontinued from the study or when subjects completed the study. The data collected during the exit interview was summarized.

### Sample Size Determination

Assuming that the difference in change from Baseline to Week 12 in average daily normalized “On” time without troublesome dyskinesia is 1.86 hours between the investigational group and the active control group, and the common standard deviation is 2.9 hours, a sample size of 52 subjects per arm will have 90% power to detect a statistically significant difference between the 2 treatment arms with a 2-sided significance level of 0.05 (using nQuery Version 8.4.0.0). Approximately 130 subjects will be randomized assuming that approximately 20% of subjects will prematurely discontinue blinded foscarbidopa/foslevodopa for subcutaneous infusion during the Double-Blind Treatment Period. This sample size also has approximately 90% power for key secondary endpoints of change from baseline in average daily normalized “Off” time, Movement Disorder Society-Unified Parkinson's Disease Rating Scale (MDS-UPDRS) Part II score and presence of morning akinesia at Week 12 (“Off” status as the first morning symptom upon awakening).

An adequate number of subjects will be enrolled in the Oral CD/LD Stabilization Period to meet the randomization goal of the study.

### Endpoints

### Primary Endpoint

The primary efficacy endpoint is the change from Baseline to Week 12 of the Double-Blind Treatment Period in average daily normalized “On” time without troublesome dyskinesia (hours) as assessed by the PD Diary.

### Secondary Endpoints

The following key secondary and other secondary endpoints of the Double-Blind Treatment Period will be included in multiplicity adjustment of the Type I error to control the familywise error rate (FWER) at 2-side significance level of 0.05 for the entire study. These variables will be tested in the following fixed sequence as a gatekeeping procedure.
Key secondary endpoints:
   Change from Baseline to Week 12 in hours of average daily normalized “Off” time as assessed by the PD diary.
   Change from Baseline to Week 12 in Motor Aspects of Experiences of Daily Living as assessed by the MDS-UPDRS Part II score.
   Presence of morning akinesia at Week 12 (defined as reporting “Off” status as the first morning symptom upon awakening) as assessed by the PD Diary.
Other secondary endpoints included in Family-wise error rate (FWER) control:
   Change from Baseline to Week 12 in hours of average daily normalized “On” time without dyskinesia as assessed by the PD Diary.
   Change from Baseline to Final Visit in sleep symptoms as assessed by Parkinson's Disease Sleep Scale-2 (PDSS-2) total score
   Change from Baseline to Final Visit in PD-related quality of life as assessed by the Parkinson's Disease Questionnarie-39 Item (PDQ-39) summary index
   Change from Baseline to Final Visit in health-related quality of life as assessed by the EQ-5D-5L summary index
   Change from Baseline to Week 12 in median bradykinesia score (BK50) as assessed by the Parkinson's KinetiGraph™/Personal KinetiGraph™ (PKG) wearable device
   Change from Baseline to Week 12 in interquartile range of bradykinesia score (BK75-BK25) as assessed by the PKG wearable device
   Change from Baseline to Week 12 in median dyskinesia score (DK50) as assessed by the PKG wearable device
   Change from Baseline to Week 12 in interquartile range of dyskinesia score (DK75-DK25) as assessed by the PKG wearable device

### Other Efficacy Endpoints

The primary and secondary efficacy endpoints included in multiplicity adjustment of the FWER are listed in above. The additional efficacy endpoints during the Double-Blind Treatment Period are:
Percent change from Baseline to Week 12 in time of tremor and daytime somnolence as assessed by the PKG wearable device
Change from Baseline to Week 12 in MDS-UPDRS Part I score, Part Ill score, Part IV score, and total score of Parts I-III
Change from Baseline to Week 12 in average daily normalized “On” time with non-troublesome dyskinesia, and “On” time with troublesome dyskinesia as assessed by the PD Diary
Percent change from Baseline to Week 12 in average daily normalized “Off” time, “On” time without troublesome dyskinesia, “On” time without dyskinesia, “On” time with non-troublesome dyskinesia, and “On” time with troublesome dyskinesia as assessed by the PD Diary
Change from Baseline to Week 12 in average daily absolute “Off” time, “On” time without troublesome dyskinesia, “On” time without dyskinesia, “On” time with non-troublesome dyskinesia, “On” time with troublesome dyskinesia, and “Asleep” time as assessed by the PD Diary without normalizing
Change from Baseline to Final Visit in PDSS-2 domain scores
Change from Baseline to Final Visit in PDQ-39 domain scores
Change from Baseline to Final Visit in EQ-5D-5L visual analogue scale (VAS) score

### Safety Endpoints

Safety endpoints are:
Adverse events (AEs), serious AEs (SAEs) and AEs of special interest (AESIs)
Local tolerability as measured by the Infusion Site Evaluation scale
Clinical laboratory values
Vital signs
Electrocardiograms (ECG)
The Columbia-Suicide Severity Rating Scale (C-SSRS)
The Questionnaire for Impulsive-Compulsive Disorders in Parkinson's Disease-Rating Scale (QUIP-RS)

Endpoints During the Oral CD/LD Stabilization PeriodThe following variables for the Oral CD/LD Stabilization Period will be summarized:
Change from Screening value to study Baseline (prior to randomization) in average daily normalized “Off” time, “On” time without troublesome dyskinesia, “On” time without dyskinesia, “On” time with non-troublesome dyskinesia, and “On” time with troublesome dyskinesia as assessed by the PD Diary
Change from Screening value to study Baseline (prior to randomization) in PD symptoms (including tremor, bradykinesia, dyskinesia, and daytime somnolence) as assessed by the PKG wearable device

### Analysis Populations

The following analysis sets will be used for the analyses.

The Oral CD/LD Analysis Set includes all subjects who received at least 1 dose of open-label CD/LD IR tablets during the Oral CD/LD Stabilization Period. The Oral CD/LD Analysis Set will be used to summarize premature discontinuations, and adverse events during the Oral CD/LD Stabilization Period.

The Full Analysis Set (FAS) includes all randomized subjects who received any dose of the product of the present disclosure during the Double-Blind Treatment Period and who have baseline and at least 1 post-baseline observation for at least 1 efficacy assessment. The FAS will be used for all efficacy analyses unless stated otherwise. Subjects will be included in the analysis according to the treatment group to which they were randomized.

The Safety Analysis Set consists of all subjects who received any dose of foscarbidopa/foslevodopa for subcutaneous infusion during the Double-Blind Treatment Period. The Safety Analysis Set will be used for all demographic, baseline, and safety analyses unless stated otherwise. Subjects will be included in the analysis according to the foslevodopa and foscarbidopa that they actually received regardless of randomization. If a subject received both active treatments (foscarbidopa/foslevodopa for subcutaneous infusion and oral CD/LD), they will be included in the treatment group that they received during the majority (>50% cumulative) of their foscarbidopa/foslevodopa for subcutaneous infusion exposure time during the Double-Blind Treatment Period. If a subject received both active treatments for equal amounts of time, or if a subject received both forms of placebo treatment (placebo solution CSCI and oral placebo capsule) for any amount of time, the subject was included in the treatment group to which they were randomized.

### Demographic, Baseline Characteristics, and Subject Disposition

Most of the demographic, baseline characteristics, and safety data in the study are presented by dose category (low dose<1800 mg/day LD or high dose≥1800 mg/day LD) within the randomized groups of foscarbidopa/foslevodopa for subcutaneous infusion and oral CD/LD. The LD dose for each subject was based on their individual need. The categorization of each subject into low dose or high dose group are based on the actual modal (most frequent) LD total daily dose received during the Double-blind Treatment Period as recorded on subject dosing diary. The dose of foscarbidopa/foslevodopa for subcutaneous infusion is presented as LD equivalents by converting foslevodopa to LD based on molecular weight (100 mg of foslevodopa is equivalent to 71 mg of LD).

### Subject Disposition

The total number of subjects who were screened, entered Oral CD/LD Stabilization Period, randomized, and received blinded foscarbidopa/foslevodopa for subcutaneous infusion was summarized. Reasons for exclusion, including screen failure, were summarized.

A summary of subject accountability was provided where the number of subjects in each of the following categories will be summarized by dose subgroup (low dose or high dose) within each treatment group:
Subjects enrolled in the Oral CD/LD Stabilization Period
Subjects who prematurely discontinued during the Oral CD/LD Stabilization Period (all reasons and primary reason)
Subjects randomized
Subjects who took any dose of blinded foscarbidopa/foslevodopa for subcutaneous infusion
Subjects who completed the Double-Blind Treatment Period
Subjects who prematurely discontinued blinded foscarbidopa/foslevodopa for subcutaneous infusion during the Double-Blind Treatment Period (all reasons and primary reason)
Subjects who prematurely discontinued study participation during the Double-Blind Treatment Period (all reasons and primary reason)
Subjects in each analysis set

Each subject was categorized to a dose subgroup based on the modal total daily dose over the treatment period. The calculation of modal total daily dose is defined in Section titled “Analysis of Daily Levodopa Dose.”

A listing of all subjects affected by the COVID-19 related study disruption by subject number and by site ID, and a description of how the subject's participation was altered because of Covid-19:
Subjects prematurely discontinued foscarbidopa/foslevodopa for subcutaneous infusion
Subjects prematurely discontinued from study
foscarbidopa/foslevodopa for subcutaneous infusion interrupted
Subjects missed a visit
Subjects conducted virtual visit

The study was conducted at 63 sites in the United States and Australia. A total of 270 subjects were screened, 174 subjects were enrolled into the Oral CD/LD Stabilization Period, 145 subjects were randomized to double-blind treatment, 141 subjects received blinded foscarbidopa/foslevodopa for subcutaneous infusion and are included in the Safety Analysis Set, and 110 subjects completed the study.

### Duration and Compliance

For the Safety Analysis Set, duration of treatment was summarized by dose subgroup (low dose or high dose) within each treatment group and for both treatment groups combined. Duration (days) of the double-blind treatment was defined for each subject as last dose date minus first dose date plus 1. Duration of treatment was summarized using the number of subjects treated, mean, standard deviation, median, minimum, and maximum. In addition, the number and percentage of subjects in each treatment duration interval (1 to 7, 8 to 14, 15 to 28, 29 to 56, and >56 days) was summarized.

For each subject, percent treatment compliance for both foscarbidopa/foslevodopa for subcutaneous infusion and foslevodopa and foscarbidopa capsules will be calculated and is defined as:
foslevodopa and foscarbidopa solution (foslevodopa and foscarbidopa CSCI or placebo solution CSCI): calculated as the number of hours the pump is infusing relative to the 24-hour/day goal and averaged over valid dosing diary days
evodopa and carbidopa capsules (placebo capsules or encapsulated CD/LD 25/100 mg IR tablets): calculated as the actual levodopa and carbidopa capsules taken relative to the number prescribed and averaged over valid dosing diary days

A valid dosing diary day was defined as one in which the pump infuses (foslevodopa and foscarbidopa or placebo solution) 80% of the entire 24-hour period (i.e., 19.2 hours).

Percent treatment compliance was summarized for the entire Double-Blind Treatment Period by treatment group and for both groups combined for the Safety Analysis Set using the number of subjects with non-missing observations, mean, standard deviation, median, minimum, and maximum.

Rescue medication was defined as the use of open-label CD/LD IR tablets while the subjects were on blinded foscarbidopa/foslevodopa for subcutaneous infusion during the Double-Blind Treatment Period. The average daily levodopa dose from blinded foscarbidopa/foslevodopa for subcutaneous infusion, from rescue medication, and the total of blinded foscarbidopa/foslevodopa for subcutaneous infusion and rescue medication was calculated for each visit that the dosing diary is collected. The average daily levodopa equivalent dose (LED) from all PD medications (blinded foscarbidopa/foslevodopa for subcutaneous infusion, loading dose, rescue medication, and concomitant PD medications) was also calculated using conversion factors for each PD medication (Tomlinson et al 2010) for each visit that the dosing diary is collected. The average daily levodopa dose and average daily LED was summarized by treatment group and for both groups combined for the Safety Analysis Set using the number of subjects with non-missing dosing diaries, mean, standard deviation, median, minimum, and maximum.

### Demographic and Baseline Clinical Characteristics, Medical History, Prior/Concomitant Medications and Dosing

Demographic and baseline clinical characteristics, medical history, and prior and concomitant medications were summarized by dose subgroup (low dose or high dose) within each treatment group and the Safety Analysis Set overall. Each subject was categorized to a dose subgroup based on the modal total daily dose over the treatment period. The calculation of modal total daily dose is defined in Section titled “Analysis of Daily Levodopa Dose.”

Categorical variables were summarized with the number and percentage of subjects; percentages will be calculated based on the number of non-missing observations. Continuous variables were summarized with descriptive statistics (number of non-missing observations, mean and standard deviation, median, minimum, and maximum).

A majority of subjects were male (70.2%), and a majority of subjects were white (92.9%). The overall mean and median age were 66.4 and 68 years, respectively. The demographics and clinical characteristics were generally balanced between treatment groups.

### Demographic and Baseline Clinical Characteristics

Continuous demographic and clinical variables are age, weight, height, body mass index (BMI), Mini-Mental State Examination (MMSE) score, baseline levodopa dose, and baseline LED. Categorical demographic and clinical variables are sex, ethnicity, race, age category (<50, 50 to <65, 65 to <75, or 75 years), BMI (<18.5, 18.5-<25.0, 25.0-<30.0, ≥30.0), country, tobacco user (current, former, never, unknown), alcohol user (current, former, never, unknown), and all Brief Neurological Examination variables.

### Medical History

Medical history data will be coded using the Medical Dictionary for Regulatory Activities (MedDRA). The actual version of the MedDRA used was specified in the statistical tables and clinical study report. The number and percentage of subjects in each medical history category (by MedDRA system organ class [SOC] and preferred term [PT]) was summarized overall and by treatment group. The SOC was presented in alphabetical order, and PTs were presented in alphabetical order within each SOC. Subjects who reported more than one condition/diagnosis were counted only once in each row (SOC or PT).

Parkinson's Disease history was summarized with the following variables:
Continuous variables: age at PD onset (years), duration of PD since symptom onset (years), age at PD diagnosis (years), duration of PD since diagnosis (years), age at onset of motor fluctuation (years), duration since onset of motor fluctuation (years).
Categorical variables: duration of PD since diagnosis (<10 years, 10 years), history of levodopa induced dyskinesia (Yes, No), levodopa response for more than 5 years (Yes, No), and Hoehn and Yahr Stage.

### Prior and Concomitant Medications

Prior and concomitant PD medications and non-PD medications were summarized separately. A prior medication is defined as any medication taken prior to the start of the Oral CD/LD Stabilization Period. A concomitant medication during the Double-Blind Treatment Period is defined as any medication that started prior to the date of the first dose of blinded foscarbidopa/foslevodopa subcutaneous infusion and continued to be taken after the first dose of foscarbidopa/foslevodopa subcutaneous infusion or any medication that started on or after the date of the first dose of foscarbidopa/foslevodopa subcutaneous infusion, but not after the date of the last dose of foscarbidopa/foslevodopa subcutaneous infusion. The number and percentage of subjects taking medications were summarized by ATC level 3, ATC level 4, and by generic drug name based on the World Health Organization (WHO) Drug Dictionary for both prior and concomitant PD medications. The number and percentage of subjects taking medications were summarized by ATC level 3 and by generic drug name based on the WHO Drug Dictionary for non-PD medications.

PD medications used at Screening (on the day prior to the start of open-label foscarbidopa/foslevodopa subcutaneous infusion of the Oral CD/LD Stabilization Period) and at Baseline (on Day −2) were summarized by the number of classes of PD medications taken for all subjects overall and by treatment group.

### Analysis of Dosing

Based on molecular weight, 100 mg of foslevodopa is equivalent to 71 mg of levodopa. Unless otherwise stated, all foslevodopa and foscarbidopa doses were presented as LD equivalents.

Unless otherwise stated, all analyses of dosing diary data were performed on valid dosing diary days.

### Analysis of Daily Prescribed Dose

Summary statistics (mean, SD, median, minimum, and maximum) were provided for prescribed dose information, including loading dose and continuous infusion rates for the investigation group, and oral levodopa dose (from encapsulated CD/LD IR) for the active control group.

When available, the summary statistics will be provided at initial prescription, first maintenance prescription following the Optimization Phase, final prescription in the 8-week Maintenance Phase, and final prescription in study. Change from initial to first maintenance, initial to maintenance final, initial to final and maintenance first to final will also be summarized.

### Analysis of Initial Optimization

Duration of initial optimization of pump infusion rate is defined as the number of days from Day 1 to the first day where there is no change to the infusion rate setting on the pump for at least 7 days.

Duration of initial optimization will be summarized by the following interval (days): 1, 2, 3, 4, 5, 6, 7, 8-14, 15-21, 22-28, and >28. When applicable, the summary statistics include mean, SD, median, minimum, and maximum.

### Analysis of Daily Levodopa Dose

Daily levodopa dose was summarized for blinded foscarbidopa/foslevodopa subcutaneous infusion and open-label oral CD/LD IR tablet at each protocol defined visit. Summary statistics are mean, SD, median, minimum, and maximum.

The modal total daily levodopa dose during the study was determined for each subject by first assigning the subject's total daily levodopa doses to narrow dose ranges in 100-mg increments (e.g., 1000 to <1100 mg, 1100 to <1200 mg) and then selecting the most frequent narrow dose range for the subject. If 2 or more dose ranges are of the same highest frequency, the highest dose range was selected.

The modal continuous pump infusion rate for the foscarbidopa/foslevodopa subcutaneous infusion treatment group was similarly defined for each subject.

The modal total daily dose was summarized for the Safety Analysis Set. The modal pump infusion rate was summarized for the foscarbidopa/foslevodopa subcutaneous infusion treatment group in the Safety Analysis Set.

### Other Dosing Analysis

The frequency of subjects taking rescue medication will be summarized by 0 time, 1 time, 2 times and ≥3 times categories and by the following study day intervals: 1-7, 8-14, 15-21, 22-28, 29-42, 43. In addition, the number of subjects with at least one occurrence and the total number of occurrences were summarized by mean, SD, median, minimum, and maximum.

### Modal (Most Frequent) Dose

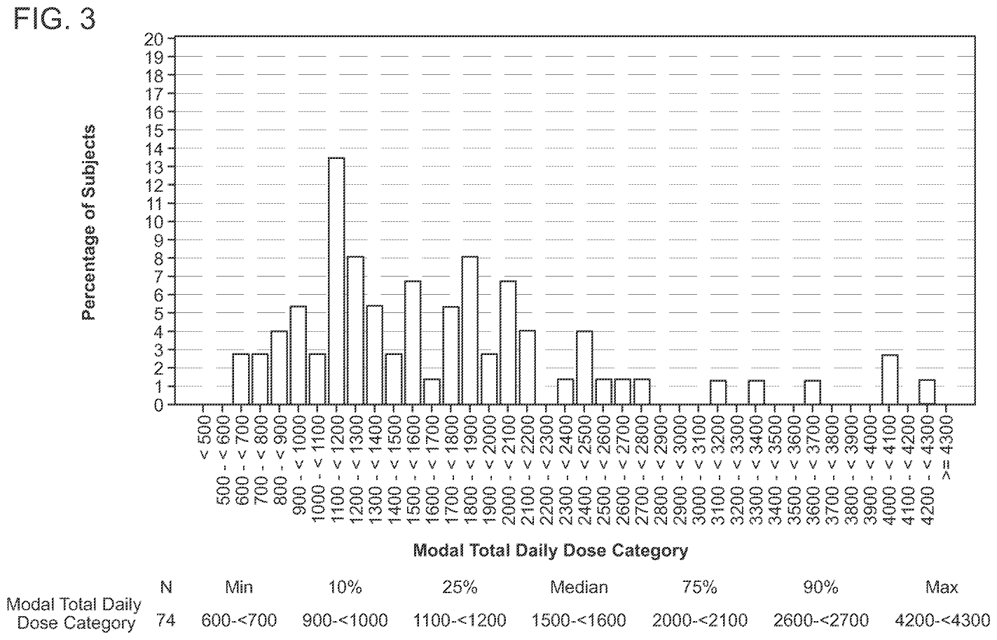

FIGS. 2 and 3 present the distribution of the modal total daily dose of levodopa for the 2 treatment groups. The median total daily dose was 1000-1100 mg for the Oral CD/LD group and 1500-1600 mg for the foscarbidopa/foslevodopa subcutaneous infusion group (Table 1).

TABLE 1
Summary of Modal Daily Dose: Safety Analysis Set

| | | foscarbidopa/foslevodopa |
|---|---|---|
| | Oral CD/LD | subcutaneous infusion |
| Modal Daily Dose (mg LD) | (N = 67) | (N = 74) |
| Min | <500 | 600-<700 |
| 10% | 500-<600 | 900-<1000 |
| 25% | 600-<700 | 1100-<1200 |
| Median | 1000-<1100 | 1500-<1600 |
| 75% | 1500-<1600 | 2000-<2100 |
| 90% | 2200-<2300 | 2600-<2700 |
| Max | 2700-<2800 | 4200-<4300 |

### Other Dosing Analysis

The frequency of subjects taking rescue medication was summarized by 0 time, 1 time, 2 times and ≥3 times categories and by the following study day intervals: 1-7, 8-14, 15-21, 22-28, 29-42, ≥43. In addition, the number of subjects with at least one occurrence and the total number of occurrences was summarized by mean, SD, median, minimum, and maximum.

### Efficacy

### General Considerations

All efficacy analyses were conducted on the FAS. All tests were 2-sided at an alpha level of 0.05.

The Primary Analysis was performed after all subjects had completed the Double-Blind Treatment Period and the database had been locked.

Unless otherwise specified, continuous variables were analyzed using the Mixed-Effect Model Repeat Measurement (MMRM) method.

Baseline for PD Diary variables was the average of the 3 valid diaries completed before Day −1. Baseline for all efficacy measures, other than the PD Diary, was defined as the last non-missing observation that is before the day of initiation of double-blinded foscarbidopa/foslevodopa subcutaneous infusion.

Post-baseline efficacy and safety measures, other than AE and Infusion Site Evaluation, collected more than 1 day after the last dose of double-blind foscarbidopa/foslevodopa subcutaneous infusion were not used for efficacy and safety analyses because data after discontinuation of foscarbidopa/foslevodopa subcutaneous infusion may be confounded by the various PD treatments that the subjects may be on after discontinuing study treatment.

Variables Derived from PD Diary

The primary, two of the key secondary, and several other secondary efficacy variables were obtained from the PD Diary. On PD Diary recording days, the subject was instructed to make an entry upon awakening from time asleep and every 30 minutes during their normal waking time for a full 24-hour period of each day from 12:00 am to 11:30 pm (48 entries, with each entry representing 0.5 hours). Each entry could be in one of 5 categories: Asleep, “Off,” “On” without dyskinesia, “On” with non-troublesome dyskinesia, and “On” with troublesome dyskinesia. For each diary day, the absolute time spent in each category was summed. The daily awake time was the sum of the absolute time spent in the 4 non-asleep categories. Daily “Off” and “On” times were normalized to a typical waking day (16 hours) to account for different sleep patterns across subjects, e.g., Normalized “On” time without troublesome dyskinesia=(Absolute “On” time without troublesome dyskinesia/Awake time)×16.

Daily normalized “Off” and “On” times are averaged over valid PD Diary days for each visit to obtain the average daily normalized “Off” and “On” times. A valid PD Diary day is defined in Section titled “Handling of Missing Items in Efficacy Instruments.”

First morning status upon awakening is determined by examining the PD Diary entries between 0:00 am and 12:00 pm on the last valid PD Diary day. First morning status upon awakening is defined as the first non-sleep and non-missing entry on the PD Diary after at least 4 consecutive entries of “Asleep” (i.e., at least 2 hours of continuous sleep).

### Handling of Missing Data:

### PD Diary

A valid PD Diary day is defined as one within 7 days prior to a clinical visit but not on or after the day of the visit and with no more than 2 hours of missing data (4 or fewer missing 30-minute entries) for the entire 24-hour diary.

For Baseline, a valid PD Diary day also cannot be on the day prior to the Randomization Visit (V6) because the subjects are asked not to take any PD medications for at least 12 hours before V6.

An invalid PD Diary day was not used in the calculation of the average daily normalized or absolute “Off” or “On” times for the visit it is associated with.

If more than 3 valid PD Diary days were available for Baseline or post-baseline visits, the 3 days closest to the clinical visit were used. If only 2 valid PD Diary days were available prior to a clinic visit, data from the 2 days were used to calculate the average daily normalized “Off” or “On” times. If only 1 valid PD Diary day was available, the value from the 1 valid PD Diary day was the visit value. If no valid PD Diary day was available for a visit, the average daily normalized “Off” or “On” times was missing for that visit.

### MDS-UPDRS

The MDS-UPDRS total score and score of each part was calculated as long as no more than 15% of the answers are missing for that assessment. The missing item was imputed as the average of the non-missing items from the same MDS-UPDRS assessment. Imputation for Part I, Part II, Part Ill, or Part IV scores used the non-missing items within the particular part, but the imputation for the total score of Parts I-III used the non-missing items from all 59 items across the 3 parts.

### PDSS-2

There was no imputation of missing responses for the PDSS-2. If any item score was missing, the total score and the corresponding domain score was not calculated.

### PDQ-39

The PDQ-39 summary index was calculated as long as no more than 15% (i.e., 5) of the answers were missing for that assessment. It was imputed as the average of the non-missing items from the same PDQ-39 assessment. The domain score was only calculated if all the questions were answered.

### EQ-5D-5L

The EQ-5D-5L summary index was only calculated if answers were provided for all 5 individual questions. The EQ-5D-5L VAS is a single value collected and there is no imputation if the VAS value is missing.

### Handling of Intercurrent Events and Missing Visit Values in Efficacy Endpoints

Intercurrent events and missing data will be handled using the following methods for efficacy analyses:
The primary approach for handling missing data for all efficacy endpoints used Mixed-Effect Model Repeat Measurement (MMRM). All observations during the period from Day 1 to 1 day after the last dose of double-blind foscarbidopa/foslevodopa subcutaneous infusion (regardless of rescue medication use during this time period) were included. Rescue medication is defined in Section titled “foslevodopa and foscarbidopa Duration and Compliance.” The repeated measures analysis was conducted using a mixed model including observed measurements at all visits. The mixed model included the categorical fixed effects of treatment, country and visit, treatment-by-visit and treatment-by-baseline interactions, and the continuous fixed covariate of baseline measurement. An unstructured variance covariance matrix was used. MMRM was the primary approach in the analysis of continuous variables in the Double-Blind Treatment Period.

### Primary Efficacy Endpoint Analyses

The primary analysis will use MMRM and include data on change from Baseline to each post-baseline visit of the Double-Blind Treatment Period in average daily normalized “On” time without troublesome dyskinesia obtained from the PD Diary. The mixed model includes the categorical fixed effects of treatment, country and visit, treatment-by-visit and treatment-by-baseline interactions, and the continuous fixed covariate of baseline measurement. Although randomization is stratified by study site, the MMRM model will not include the “site” effect due to the large number of sites planned compared to the number of subjects that will be randomized in the study. If the final number of sites that randomized at least 1 subject is less than or equal to 30, the “site” effect may be added to the model. The primary approach for handling intercurrent events and missing data will be based on the MMRM method as specified in Section titled “Handling of Intercurrent Events and Missing Visit Values in Efficacy Endpoints.”

An unstructured variance covariance matrix will be used. Due to the short half-life (˜1.5 hours) of foscarbidopa/foslevodopa subcutaneous infusion, the compound symmetry variance covariance matrix will be used if the model does not converge with unstructured matrix. Parameter estimation is based on the REML method. The primary comparison will be the contrast on change from Baseline between the investigational and active control groups at Week 12.

Change from Baseline in average daily normalized “On” time without troublesome dyskinesia conducted on MMRM will be displayed by investigational group and active control group in a figure.

The proportion of subjects who met average daily normalized ‘On’ time without troublesome dyskinesia responder criteria at Week 12 will be summarized by investigational group and active control group at the thresholds from 0% to 100% with 10% as an increment. The P-value for test difference in the distribution between the 2 groups will be provided by conducting the Monte Carlo exact Kolmogorov-Smirnov test. The percentage of subjects who achieved at least one certain percent reduction in average daily normalized “On” time without troublesome dyskinesia at endpoint will be displayed by investigational group and active control group in a figure. The same analysis will also be conducted for average daily normalized “Off” time.

### Additional Analyses of the Primary Efficacy Endpoints

Two sensitivity analyses will be conducted on the primary efficacy endpoint to account for missing data.
Sensitivity analysis #1: A “jump-to-reference” analytic approach to account for missing data due to subjects who prematurely discontinue blinded foscarbidopa/foslevodopa subcutaneous infusion during the Double-Blind Treatment Period will be implemented as described in Section titled “Handling of Intercurrent Events and Missing Visit Values in Efficacy Endpoints.”
Sensitivity analysis #2: The change from Baseline to the last available value in average daily normalized “On” time without troublesome dyskinesia will be analyzed using an ANCOVA model with the categorical fixed effects of treatment and country, and baseline score as a covariate. If the final number of sites that randomized at least 1 subject is less than or equal to 30, the “site” effect may be added to the model. Missing Week 12 data will be handled using the last available value approach as specified in Section titled “Handling of Intercurrent Events and Missing Visit Values in Efficacy Endpoints.”

A summary of the primary and 2 sensitivity analyses of the primary efficacy endpoint is presented in Error! Reference source not found.

TABLE 2
Summary of Primary and Sensitivity Analysis for the Primary
Efficacy Endpoint of Change from Baseline to Week 12 in Average
Daily Normalized “On” Time Without Troublesome Dyskinesia

| | Dependent | Data | Analysis |
|---|---|---|---|
| Analysis | Variable | Included | Method |
| Primary | Change from | During the period from Day | MMRM with categorical fixed |
| Analysis | baseline to | 1 to 1 day after the last | effects of treatment, country and |
| | post-baseline | dose of double-blind | visit, treatment-by-visit and |
| | visits through | foscarbidopa/foslevodopa | treatment-by-baseline |
| | Week 12 | subcutaneous infusion, | interactions, and the continuous |
| | | regardless of rescue | fixed covariate of baseline |
| | | medication use during this | measurement. Missing data will |
| | | time period. | be handled using MMRM as |
| | | | specified in Section titled |
| | | | “Handling of Intercurrent Events |
| | | | and Missing Visit Values in |
| | | | Efficacy Endpoints.” |
| Sensitivity | | | Jump-to-Reference will be used |
| Analysis | | | to handle missing data in the |
| #1 | | | investigational arm. The details |
| | | | for this approach are described |
| | | | in Section titled “Handling of |
| | | | Intercurrent Events and Missing |
| | | | Visit Values in Efficacy |
| | | | Endpoints.” |
| Sensitivity | Change from | | ANCOVA model with the |
| Analysis | baseline to | | categorical fixed effects of |
| #2 | the Week 12 | | treatment and country, and |
| | visit | | baseline score as a covariate. |
| | | | Missing Week 12 data will be |
| | | | imputed using last available |
| | | | value. |

### Secondary Efficacy Analyses

### Key Secondary and Other Secondary Efficacy Analyses

Key secondary efficacy endpoints and other secondary endpoints included in FWER control are described in the Section titled “Secondary Endpoints.”

Change from Baseline to Week 12 in average daily normalized “Off” time, MDS-UPDRS Part II score, average daily normalized “On” time without dyskinesia, and PKG variables will be analyzed using the same MMRM model as the primary efficacy analysis.

Change from Baseline to Final Visit in the PDSS-2 total score, PDQ-39 summary index, and EQ-5D-5L summary index will be analyzed using an ANCOVA model with categorical fixed effects of treatment and country, and baseline score as a covariate. If the final number of sites that randomized at least 1 subject is less than or equal to 30, the “site” effect may be added to the model.

The percent of subjects in each category of first morning status upon awakening (“Off,” “On” without dyskinesia, “On” with non-troublesome dyskinesia, and “On” with troublesome dyskinesia) will be presented by visit and treatment group.

The first morning status upon awakening (“Off” or not “Off”) on the last valid PD Diary day at each post-baseline visit will be analyzed using a generalized linear mixed model (GLMM) with a logit link function to compare the probability of having morning akinesia between the treatment groups. The model will include fixed, categorical effects of treatment, country, visit, treatment-by-visit interaction, and baseline first morning status upon awakening. An unstructured variance covariance matrix will be used. The compound symmetry variance covariance matrix will be used if the model does not converge with unstructured matrix. The odds ratio for treatment comparison at Week 12 and their associated 95% CIs will be estimated from the GLMM.

### Other Efficacy Analyses

Other supportive efficacy variables are described in Section titled “Other Efficacy Endpoints”. Variables derived from the PD diary, MDS-UPDRS, and PKG wearable device will be analyzed using the same MMRM model as the primary efficacy analysis. Variables derived from the PDSS-2, PDQ-39, and EQ-5D-5L will be analyzed with the same ANCOVA model described in Section titled “Key Secondary and Other Secondary Efficacy Analyses.”

Distribution of time spent in different motor symptoms based on PD Diary data will be displayed in pie charts for the Baseline Visit and the Week 12 Visit.

### Efficacy Analyses During the Oral CD/LD Stabilization Period

Additional efficacy variables for the Oral CD/LD Stabilization Period are described in Section titled Safety Endpoints. These variables will be summarized by investigational and active control groups and for all subjects in the FAS. No statistical testing will be performed.

### Efficacy Subgroup Analyses

Subgroup analyses of change from Baseline to Final Visit of the Double-Blind Treatment Period in average daily normalized “On” and “Off” times will be conducted for the following subgroups:
Age category (<65 years or 65 years)
Sex (male or female)
Race (white or other)
Country (US or Australia)
Duration of PD (time since diagnosis to randomization) (<10 years or ≤10 years)
Concomitant dopamine agonist use (yes or no)
Dose category (low or high levodopa dose). Each subject will be categorized to a dose subgroup based on the modal total daily dose over the treatment period. The calculation of modal total daily dose is defined in Section Error! Reference source not found. “Analysis of Daily Levodopa Dose.”

A subgroup analysis will be conducted on dose subgroup for the key secondary and other secondary endpoints described in Section titled “Secondary Endpoints” except for the variables that were derived from the PD diaries, which were analyzed by all subgroups described in the above paragraph.

A subgroup analysis may not be conducted if one stratum of the subgroup variable comprises of <20% of the FAS Analysis Set.

Subgroup analyses was performed on the FAS using an ANCOVA model with the terms of treatment, subgroup variable, the treatment-by-subgroup variable interaction, and Baseline as a covariate. The statistical comparison of the investigational and active control groups within each subgroup stratum was performed when the statistical significance of the treatment-by-subgroup interaction term was achieved at 0.100 level.

### Results

A multiple testing procedure was used to provide strong control of the Type I error rate at alpha=0.05 (2-sided) across analyses comparing the foscarbidopa/foslevodopa subcutaneous infusion group to the Oral CD/LD group with respect to the primary efficacy endpoint and ranked secondary endpoints. Specifically, testing utilized a sequence of hypothesis testing for the primary endpoint followed by the ranked secondary endpoints. Treatment with foscarbidopa/foslevodopa subcutaneous infusion resulted in statistically significant and clinically meaningful improvements compared with oral CD/LD in the primary efficacy endpoint and the first key secondary endpoint.

TABLE 3
Primary, Key Secondary and Other Secondary Endpoints Included
in the Multiplicity Control: Full Analysis Set

| | | | | | | |
|---|---|---|---|---|---|---|
| | | Base- | | LS | LS Mean | |
| | | line | Mean | Mean | of Dif- | |
| Treatment | | Mean | Change | Change | ference | P |
| Group | N | (SD) | (SD) | (SE) | (SE) | value |

Primary: Change from Baseline to Week 12 in hours of average
daily normalized “On” time without troublesome dyskinesia^{a}

| | | | | | | |
|---|---|---|---|---|---|---|
| Oral CD/LD | 67 | 9.49 | 0.85 | 0.97 | | |
| | | (2.62) | (3.46) | (0.50) | | |
| foscarbidopa/ | 73 | 9.20 | 3.36 | 2.72 | 1.75 | 0.0083 |
| foslevodopa | | (2.42) | (3.62) | (0.52) | (0.65) | |
| subcutaneous | | | | | | |
| infusion | | | | | | |

Key Secondary #1: Change from Baseline to Week 12 in hours of
average daily normalized “Off” time^{a}

| | | | | | | |
|---|---|---|---|---|---|---|
| Oral CD/LD | 67 | 5.91 | −0.93 | −0.96 | | |
| | | (1.88) | (3.31) | (0.49) | | |
| foscarbidopa/ | 73 | 6.34 | −3.41 | −2.75 | −1.79 | 0.0054 |
| foslevodopa | | (2.27) | (3.76) | (0.50) | (0.63) | |
| subcutaneous | | | | | | |
| infusion | | | | | | |

Key Secondary #2: Change from Baseline to Week 12
MDS-UPDRS Part II score^{a}

| | | | | | | |
|---|---|---|---|---|---|---|
| Oral CD/LD | 67 | 13.27 | −0.48 | −1.06 | | |
| | | (6.37) | (4.91) | (0.79) | | |
| foscarbidopa/ | 74 | 15.31 | −3.76 | −2.65 | −1.58 | 0.1318 |
| foslevodopa | | (6.93) | (6.98) | (0.82) | (1.05) | |
| subcutaneous | | | | | | |
| infusion | | | | | | |

| | | | | | |
|---|---|---|---|---|---|
| | | | | LS Mean of | |
| | | | | Odds Ratio | |
| Treatment | | Baseline | Week 12 | (SE) at | P |
| Group | N | n/N (%) | n/N (%) | Week 12 | value |

Key Secondary #3: Presence of morning Akinesia at
Week 12^{b} (defined as reporting “Off” status as the
first morning symptom upon awakening)

| | | | | | |
|---|---|---|---|---|---|
| Oral CD/LD | 67 | 51/67 | 38/60 | | |
| | | (76.1) | (63.3) | | |
| foscarbidopa/ | 71 | 56/71 | 8/47 | 0.12 | ≤0.001^{d} |
| foslevodopa | | (78.9) | (17.0) | (0.49) | |
| subcutaneous | | | | | |
| infusion | | | | | |

| | | | | | | |
|---|---|---|---|---|---|---|
| | | | | | LS | |
| | | Base- | | LS | Mean | |
| | | line | Mean | Mean | of Dif- | |
| Treatment | | Mean | Change | Change | ference | P |
| Group | N | (SD) | (SD) | (SE) | (SE) | value |

Change from Baseline to Week 12 in hours of average daily
normalized “On” time without dyskinesia^{a}

| | | | | | | |
|---|---|---|---|---|---|---|
| Oral CD/LD | 67 | 7.47 | 1.15 | 1.32 | | |
| | | (3.72) | (3.63) | (0.53) | | |
| foscarbidopa/ | 73 | 7.23 | 3.96 | 3.13 | 1.81 | 0.0091^{d} |
| foslevodopa | | (3.14) | (3.77) | (0.54) | (0.68) | |
| subcutaneous | | | | | | |
| infusion | | | | | | |

Change from Baseline to Final Visit in PDSS-2 total score^{c}

| | | | | | | |
|---|---|---|---|---|---|---|
| Oral CD/LD | 59 | 18.71 | −1.64 | −2.52 | | |
| | | (8.77) | (6.22) | (1.12) | | |
| foscarbidopa/ | 44 | 21.70 | −8.34 | −7.92 | −5.40 | ≤0.001^{d} |
| foslevodopa | | (9.04) | (8.86) | (1.18) | (1.32) | |
| subcutaneous | | | | | | |
| infusion | | | | | | |

Change from Baseline to Final Visit in PDQ-39 summary index^{c}

| | | | | | | |
|---|---|---|---|---|---|---|
| Oral CD/LD | 59 | 26.52 | −0.57 | −2.28 | | |
| | | (13.89) | (8.79) | (1.75) | | |
| foscarbidopa/ | 45 | 29.31 | −5.66 | −6.38 | −4.10 | 0.0470^{d} |
| foslevodopa | | (15.84) | (13.71) | (1.83) | (2.04) | |
| subcutaneous | | | | | | |
| infusion | | | | | | |

Change from Baseline to Final Visit in EQ-5D-5L summary index^{c}

| | | | | | | |
|---|---|---|---|---|---|---|
| Oral CD/LD | 59 | 0.747 | 0.014 | 0.002 | | |
| | | (0.125) | (0.128) | (0.021) | | |
| foscarbidopa/ | 44 | 0.748 | 0.061 | 0.051 | 0.049 | 0.0566 |
| foslevodopa | | (0.131) | (0.164) | (0.022) | (0.025) | |
| subcutaneous | | | | | | |
| infusion | | | | | | |

Change from Baseline to Week 12 in median bradykinesia score
(BK50) as assessed by the PKG wearable device^{a}

| | | | | | | |
|---|---|---|---|---|---|---|
| Oral CD/LD | 59 | 26.63 | −0.36 | −0.34 | | |
| | | (7.68) | (3.32) | (0.52) | | |
| foscarbidopa/ | 66 | 25.97 | 1.26 | 1.38 | 1.72 | 0.0184^{d} |
| foslevodopa | | (7.65) | (3.98) | (0.56) | (0.72) | |
| subcutaneous | | | | | | |
| infusion | | | | | | |

Change from Baseline to Week 12 in interquartile range of bradykinesia
score (BK75-BK25) as assessed by the PKG wearable device a

| | | | | | | |
|---|---|---|---|---|---|---|
| Oral CD/LD | 59 | 17.80 | −0.02 | 0.13 | | |
| | | (4.26) | (3.76) | (0.49) | | |
| foscarbidopa/ | 66 | 17.13 | 0.01 | 0.31 | 0.18 | 0.7989 |
| foslevodopa | | (4.18) | (3.50) | (0.54) | (0.69) | |
| subcutaneous | | | | | | |
| infusion | | | | | | |

Change from Baseline to Week 12 in median dyskinesia score
(DK50) as assessed by the PKG wearable device^{a}

| | | | | | | |
|---|---|---|---|---|---|---|
| Oral CD/LD | 59 | 3.70 | 1.58 | 1.02 | | |
| | | (5.45) | (13.53) | (1.38) | | |
| foscarbidopa/ | 66 | 4.46 | −2.36 | −1.71 | −2.73 | 0.1656 |
| foslevodopa | | (10.14) | (6.97) | (1.41) | (1.96) | |
| subcutaneous | | | | | | |
| infusion | | | | | | |

Change from Baseline to Week 12 in interquartile range of dyskinesia
score (DK75-DK25) as assessed by the PKG wearable device^{a}

| | | | | | | |
|---|---|---|---|---|---|---|
| Oral CD/LD | 59 | 11.31 | 3.30 | 2.72 | | |
| | | (16.61) | (25.67) | (2.59) | | |
| foscarbidopa/ | 66 | 12.31 | −3.79 | −2.77 | −5.49 | 0.1347 |
| foslevodopa | | (20.36) | (9.18) | (2.64) | (3.65) | |
| subcutaneous | | | | | | |
| infusion | | | | | | |

ANCOVA = analysis of covariance;
CD/LD = carbidopa/levodopa;
FAS = Full Analysis Set;
GLMM = generalized linear mixed model;
MDS-UPDRS = Movement Disorder Society-Unified Parkinson's Disease Rating Scale;
MMRM = mixed-effect model for repeat measures;
PDSS-2 = Parkinson's Disease Sleep Scale 2;
PDQ-39 = Parkinson's Disease Questionnaire-39 item;
PKG = Parkinson's KinetiGraph ® or Personal KinetiGraph ® (United States);
SE = standard error
^{a}Analyzed with an MMRM.
^{b}Analyzed with a GLMM.
^{c}Analyzed with an ANCOVA model.
^{d}Even though the nominal p-value is ≤0.05, statistical significance cannot be claimed due to MDS-UPDRS Part II not being significant.

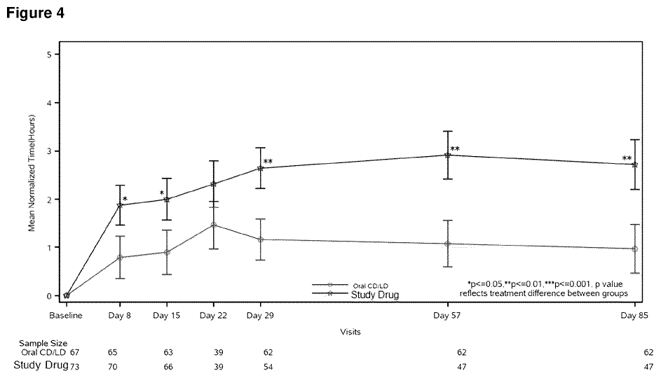

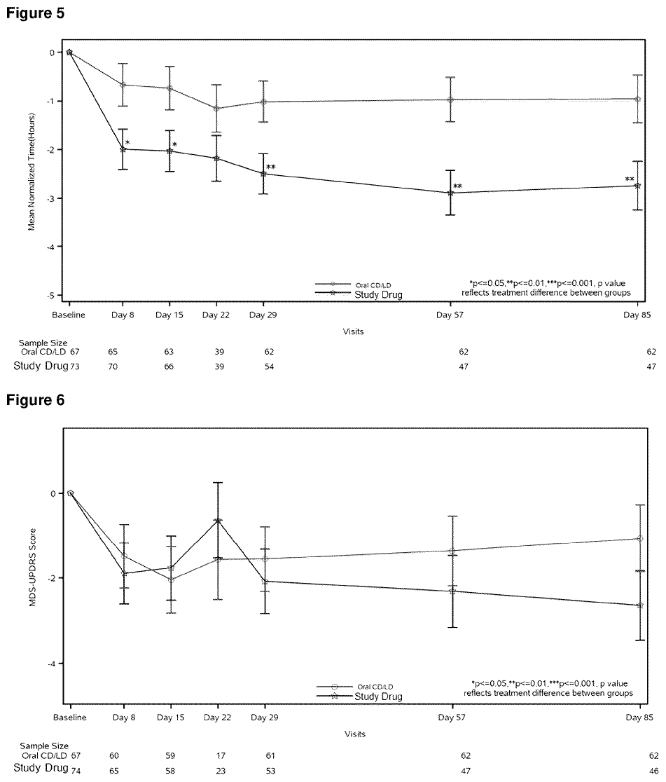

FIGS. 4-5 present the longitudinal treatment profile of the primary and first key secondary endpoint. Improvements (nominal p-value≤0.05) were observed as early as Week 1 (Day 8), the first timepoint when post-baseline PD Diary was collected and persisted through the end of the Double-Blind Treatment Period at Week 12 with the exception of Week 3 (Day 22) which was an optional visit and the sample size was lower.

FIG. 6 presents the longitudinal treatment profile of MDS-UPDRS Part II score.

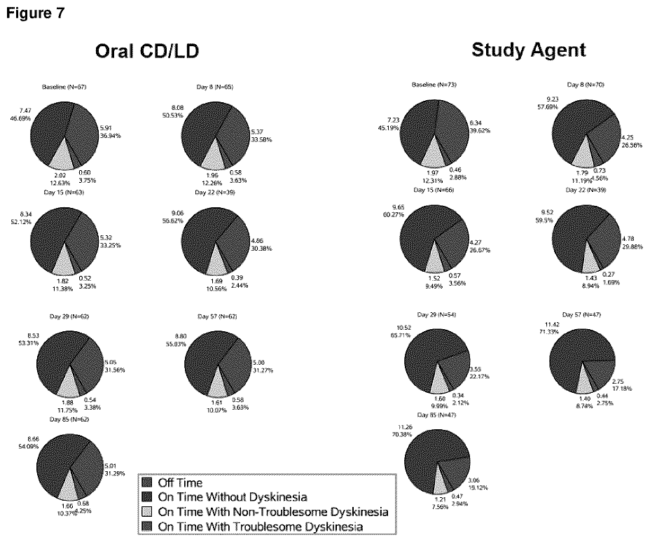

FIG. 7 showed that foscarbidopa/foslevodopa subcutaneous infusion was superior to Oral CD/LD in:
Reducing:
   “Off” time
   “On” time with troublesome dyskinesia
   Even the relatively good state of “On” time with non-troublesome dyskinesia
Complementary increases in the most desirable “On” time without dyskinesia

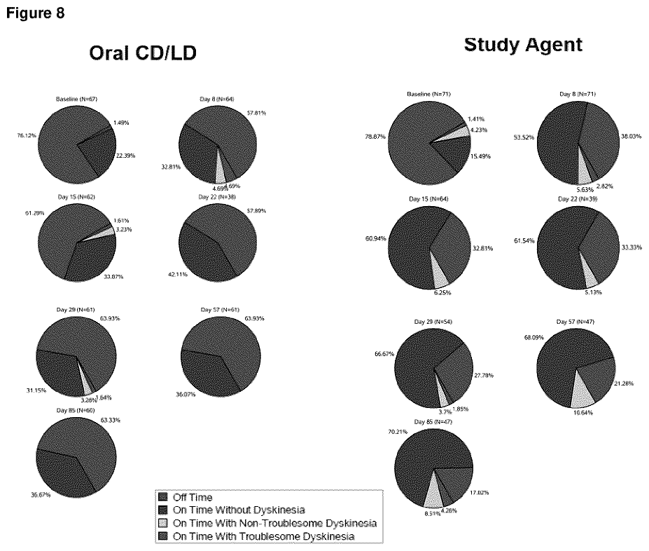

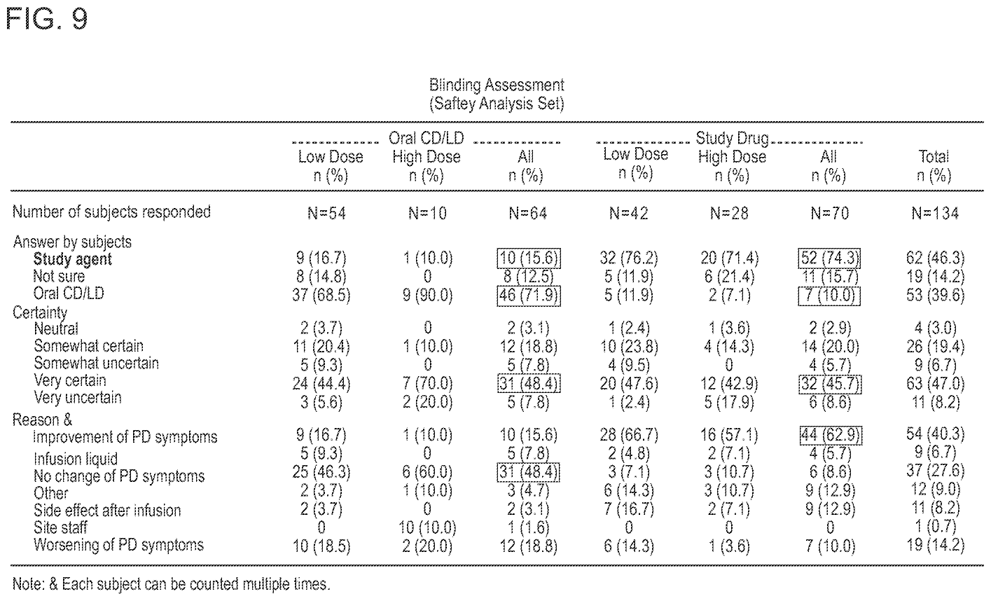

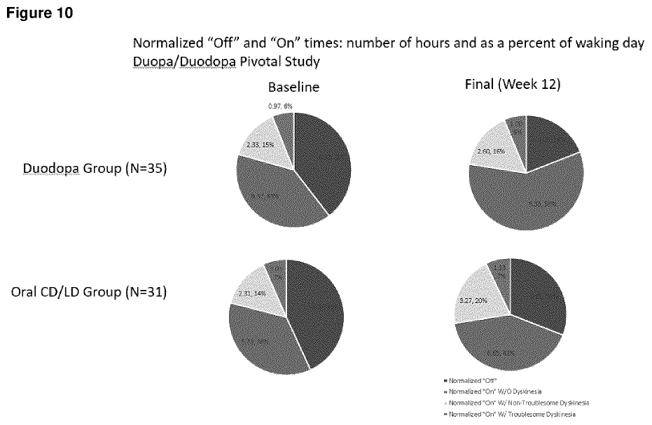

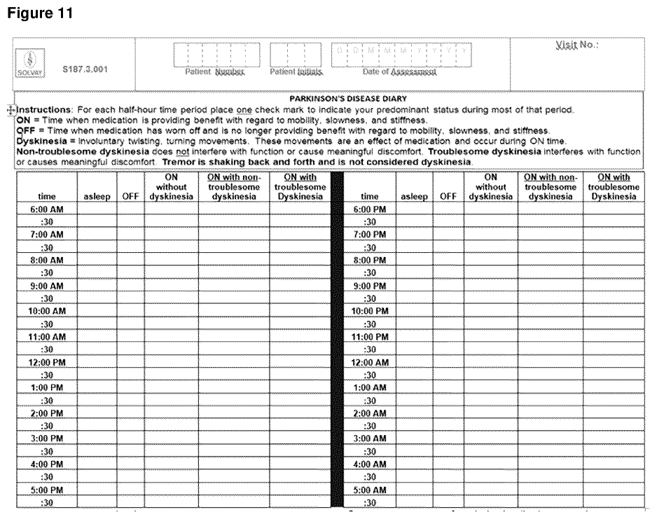

FIG. 8 demonstrated that in the foscarbidopa/foslevodopa (study agent) subcutaneous infusion group, the percentage of subjects reporting morning akinesia (awakening in “Off” state) dramatically decreased from 80% at baseline to 30% at the end of the 12-week treatment period, with 70% of the subjects reporting awakening in “On” state without dyskinesia at the end of the study.

The increase in “On” time at week 12 was 2.72 hours for the foscarbidopa/foslevodopa subcutaneous infusion versus 0.97 hours for oral levodopa/carbidopa (LD/CD) (p=0.0083). Improvements versus baseline in “On” time were observed as early as the first week and persisted throughout the 12 weeks of the study.

It was also observed that an improvement from baseline in hours of average daily normalized “Off” time followed a similar pattern with reductions versus placebo observed after the first week and persisting through Week 12. Decreases in “Off” time after 12 weeks were 2.75 hours for the foscarbidopa/foslevodopa subcutaneous infusion versus 0.96 hours for oral LD/CD (p=0.0054).

### Safety

The foscarbidopa/foslevodopa subcutaneous infusion was generally safe and well tolerated.
The systemic safety profile of foscarbidopa/foslevodopa subcutaneous infusion was generally consistent with the well-established safety profile of CD/LD drugs.
Most (>90%) of the AEs reported were non-serious and mild/moderate in severity
Incidence of SAEs was comparable between foscarbidopa/foslevodopa subcutaneous infusion and oral CD/LD drugs.
Incidence of Infusion site events was higher in the foscarbidopa/foslevodopa subcutaneous infusion group than the Oral CD/LD group.
The rates of premature discontinuations and TEAEs leading to discontinuation were higher in the foscarbidopa/foslevodopa subcutaneous infusion group than in the Oral CD/LD group.
Infusion site events observed with foscarbidopa/foslevodopa subcutaneous infusion were mostly nonserious, were mild or moderate in severity, and resolved with or without treatment.
Incidence of hallucination/psychosis events was higher in the foscarbidopa/foslevodopa subcutaneous infusion group than in the Oral CD/LD group.
Incidence of falls and associated injuries was higher in the Oral CD/LD group than the foscarbidopa/foslevodopa subcutaneous infusion group.
Incidences of other AESIs (weight loss, somnolence and polyneuropathy) were low and comparable between the two treatment groups.

TABLE 4
Adverse Reactions in Study 1 for foscarbidopa/foslevodopa subcutaneous
infusion in Patients with Advanced Parkinson's disease

| | foscarbidopa/foslevodopa | Oral immediate-release |
|---|---|---|
| | subcutaneous infusion | carbidopa-levodopa |
| | (n = 74) | (n = 67) |
| Preferred Term | % | % |
| Infusion site | 62 | 8 |
| reaction^{a} | | |
| Infusion site | 28 | 3 |
| infection^{b} | | |
| Dyskinesia | 11 | 6 |
| On and off | 8 | 0 |
| phenomenon | | |
| Fall | 8 | 18 |
| Hallucination^{c} | 12 | 2 |

### Labs, Vital Signs, ECG and C-SSRS Findings

No clinically meaningful changes from baseline or trends were observed based on the review of laboratory results, vital signs, and ECG for either treatment group, and no safety concerns were identified. There was no evidence of increased suicidality for either treatment group based on the review of C-SSRS data.

### Primary and Key Secondary Endpoints: Full Analysis Set

TABLE 5

| | | | | | | |
|---|---|---|---|---|---|---|
| | | Base- | | LS | LS Mean | |
| | | line | Mean | Mean | of Dif- | |
| Treatment | | Mean | Change | Change | ference | P |
| Group | N | (SD) | (SD) | (SE) | (SE) | value |

Primary: Change from Baseline to Week 12 in hours of average
daily normalized “On” time without troublesome dyskinesia

| | | | | | | |
|---|---|---|---|---|---|---|
| Oral CD/LD | 67 | 9.49 | 0.85 | 0.97 | | |
| | | (2.62) | (3.46) | (0.50) | | |
| foscarbidopa/ | 73 | 9.20 | 3.36 | 2.72 | 1.75 | 0.0083 |
| foslevodopa | | (2.42) | (3.62) | (0.52) | (0.65) | |
| subcutaneous | | | | | | |
| infusion | | | | | | |

Key Secondary #1: Change from Baseline to Week 12 in
hours of average daily normalized “Off” time

| | | | | | | |
|---|---|---|---|---|---|---|
| Oral CD/LD | 67 | 5.91 | −0.93 | −0.96 | | |
| | | (1.88) | (3.31) | (0.49) | | |
| foscarbidopa/ | 73 | 6.34 | −3.41 | −2.75 | −1.79 | 0.0054 |
| foslevodopa | | (2.27) | (3.76) | (0.50) | (0.63) | |
| subcutaneous | | | | | | |
| infusion | | | | | | |

Key Secondary #2: Change from Baseline to Week 12
MDS-UPDRS Part II score (Motor experiences of daily living)

| | | | | | | |
|---|---|---|---|---|---|---|
| Oral CD/LD | 67 | 13.27 | −0.48 | −1.06 | | |
| | | (6.37) | (4.91) | (0.79) | | |
| foscarbidopa/ | 74 | 15.31 | −3.76 | −2.65 | −1.58 | 0.1318 |
| foslevodopa | | (6.93) | (6.98) | (0.82) | (1.05) | |
| subcutaneous | | | | | | |
| infusion | | | | | | |

TABLE 6

| | | | | | |
|---|---|---|---|---|---|
| | | Baseline | Week 12 | Week 12 LS | |
| | | n/N | n/N | Mean of Odds | P |
| Treatment Group | N | (%) | (%) | Ratio (SE) | value |

Key Secondary #3: Presence of morning Akinesia at Week 12 (defined as reporting
“Off” status as the first morning symptom upon awakening)

| | | | | | |
|---|---|---|---|---|---|
| Oral CD/LD | 67 | 51/67 | 38/60 | | |
| | | (76.1) | (63.3) | | |
| foscarbidopa/foslevodopa | 71 | 56/71 | 8/47 | 0.12 | ≤0.001^{a} |
| subcutaneous infusion | | (78.9) | (17.0) | (0.49) | |

^{a}Even though the nominal p-value is ≤ 0.05, statistical significance cannot be claimed due to MDS-UPDRS Part II not being significant
MDS-UPDRS = Movement Disorder Society-Unified Parkinson's Disease Rating Scale, lower score indicating less impairment

### Other Secondary Endpoints Included in Multiplicity Control Full Analysis Set

TABLE 7

| | | | | | | |
|---|---|---|---|---|---|---|
| | | Base- | | LS | LS Mean | |
| | | line | Mean | Mean | of Dif- | |
| Treatment | | Mean | Change | Change | ference | |
| Group | N | (SD) | (SD) | (SE) | (SE) | P value |

Change from Baseline to Week 12 in hours of average daily
normalized “On” time without dyskinesia

| | | | | | | |
|---|---|---|---|---|---|---|
| Oral CD/LD | 67 | 7.47 | 1.15 | 1.32 | | |
| | | (3.72) | (3.63) | (0.53) | | |
| foscarbidopa/ | 73 | 7.23 | 3.96 | 3.13 | 1.81 | 0.0091^{a} |
| foslevodopa | | (3.14) | (3.77) | (0.54) | (0.68) | |
| subcutaneous | | | | | | |
| infusion | | | | | | |

Change from Baseline to Final Visit in PDSS-2 total score

| | | | | | | |
|---|---|---|---|---|---|---|
| Oral CD/LD | 59 | 18.71 | −1.64 | −2.52 | | |
| | | (8.77) | (6.22) | (1.12) | | |
| foscarbidopa/ | 44 | 21.70 | −8.34 | −7.92 | −5.40 | ≤0.001^{a} |
| foslevodopa | | (9.04) | (8.86) | (1.18) | (1.32) | |
| subcutaneous | | | | | | |
| infusion | | | | | | |

Change from Baseline to Final Visit in PDQ-39 summary index

| | | | | | | |
|---|---|---|---|---|---|---|
| Oral CD/LD | 59 | 26.52 | −0.57 | −2.28 | | |
| | | (13.89) | (8.79) | (1.75) | | |
| foscarbidopa/ | 45 | 29.31 | −5.66 | −6.38 | −4.10 | 0.0470^{a} |
| foslevodopa | | (15.84) | (13.71) | (1.83) | (2.04) | |
| subcutaneous | | | | | | |
| infusion | | | | | | |

Change from Baseline to Final Visit EQ-5D-5L summary index

| | | | | | | |
|---|---|---|---|---|---|---|
| Oral CD/LD | 59 | 0.747 | 0.014 | 0.002 | | |
| | | (0.125) | (0.128) | (0.021) | | |
| foscarbidopa/ | 44 | 0.748 | 0.061 | 0.051 | 0.049 | 0.0566 |
| foslevodopa | | (0.131) | (0.164) | (0.022) | (0.025) | |
| subcutaneous | | | | | | |
| infusion | | | | | | |

^{a}Even though the nominal p-value is ≤0.05, statistical significance cannot be claimed due to MDS-UPDRS Part II not being significant
PDSS-2 = Parkinson's Disease Sleep Scale 2, total score range 0 to 60 with lower score indicating less impairment
PDQ-39 = Parkinson's Disease Questionnaire-39 item, summary index value range 0 to 100 with lower score indicating less impairment
EQ-5D-5L = EuroQol-5dimension-5level, summary index range −0.11 to 1 with higher value indicating better quality of life

### PKG Wearable Device Endpoints Included in Multiplicity Control Full Analysis Set

TABLE 8

| | | | | | | |
|---|---|---|---|---|---|---|
| | | Base- | | | LS Mean | |
| | | line | Mean | LS Mean | of Dif- | |
| Treatment | | Mean | Change | Change | ference | P |
| Group | N | (SD) | (SD) | (SE) | (SE) | value |

Change from Baseline to Week 12 in median bradykinesia
score (BK50)

| | | | | | | |
|---|---|---|---|---|---|---|
| Oral CD/LD | 59 | 26.63 | −0.36 | −0.34 | | |
| | | (7.68) | (3.32) | (0.52) | | |
| (foscarbidopa/ | 66 | 25.97 | 1.26 | 1.38 | 1.72 | 0.0184^{a} |
| foslevodopa) | | (7.65) | (3.98) | (0.56) | (0.72) | |
| subcutaneous | | | | | | |
| infusion | | | | | | |

Change from Baseline to Week 12 in interquartile range of
bradykinesia score (BK75-BK25)

| | | | | | | |
|---|---|---|---|---|---|---|
| Oral CD/LD | 59 | 17.80 | −0.02 | 0.13 | | |
| | | (4.26) | (3.76) | (0.49) | | |
| (foscarbidopa/ | 66 | 17.13 | 0.01 | 0.31 | 0.18 | 0.7989 |
| foslevodopa) | | (4.18) | (3.50) | (0.54) | (0.69) | |
| subcutaneous | | | | | | |
| infusion | | | | | | |

Change from Baseline to Week 12 in median dyskinesia score (DK50)

| | | | | | | |
|---|---|---|---|---|---|---|
| Oral CD/LD | 59 | 3.70 | 1.58 | 1.02 | | |
| | | (5.45) | (13.53) | (1.38) | | |
| (foscarbidopa/ | 66 | 4.46 | −2.36 | −1.71 | −2.73 | 0.1656 |
| foslevodopa) | | (10.14) | (6.97) | (1.41) | (1.96) | |
| subcutaneous | | | | | | |
| infusion | | | | | | |

Change from Baseline to Week 12 in interquartile range of
dyskinesia score (DK75-DK25)

| | | | | | | |
|---|---|---|---|---|---|---|
| Oral CD/LD | 59 | 11.31 | 3.30 | 2.72 | | |
| | | (16.61) | (25.67) | (2.59) | | |
| (foscarbidopa/ | 66 | 12.31 | −3.79 | −2.77 | −5.49 | 0.1347 |
| foslevodopa) | | (20.36) | (9.18) | (2.64) | (3.65) | |
| subcutaneous | | | | | | |
| infusion | | | | | | |

Change from Baseline to Week 12 in median bradykinesia score (BK50)

| | | | | | | |
|---|---|---|---|---|---|---|
| Oral CD/LD | 59 | 26.63 | −0.36 | −0.34 | | |
| | | (7.68) | (3.32) | (0.52) | | |
| (foscarbidopa/ | 66 | 25.97 | 1.26 | 1.38 | 1.72 | 0.0184^{a} |
| foslevodopa) | | (7.65) | (3.98) | (0.56) | (0.72) | |
| subcutaneous | | | | | | |
| infusion | | | | | | |

Change from Baseline to Week 12 in interquartile range of
bradykinesia score (BK75-BK25)

| | | | | | | |
|---|---|---|---|---|---|---|
| Oral CD/LD | 59 | 17.80 | −0.02 | 0.13 | | |
| | | (4.26) | (3.76) | (0.49) | | |
| (foscarbidopa/ | 66 | 17.13 | 0.01 | 0.31 | 0.18 | 0.7989 |
| foslevodopa) | | (4.18) | (3.50) | (0.54) | (0.69) | |
| subcutaneous | | | | | | |
| infusion | | | | | | |

Change from Baseline to Week 12 in median dyskinesia score (DK50)

| | | | | | | |
|---|---|---|---|---|---|---|
| Oral CD/LD | 59 | 3.70 | 1.58 | 1.02 | | |
| | | (5.45) | (13.53) | (1.38) | | |
| (foscarbidopa/ | 66 | 4.46 | −2.36 | −1.71 | −2.73 | 0.1656 |
| foslevodopa) | | (10.14) | (6.97) | (1.41) | (1.96) | |
| subcutaneous | | | | | | |
| infusion | | | | | | |

Change from Baseline to Week 12 in interquartile range of
dyskinesia score (DK75-DK25)

| | | | | | | |
|---|---|---|---|---|---|---|
| Oral CD/LD | 59 | 11.31 | 3.30 | 2.72 | | |
| | | (16.61) | (25.67) | (2.59) | | |
| (foscarbidopa/ | 66 | 12.31 | −3.79 | −2.77 | −5.49 | 0.1347 |
| foslevodopa) | | (20.36) | (9.18) | (2.64) | (3.65) | |
| subcutaneous | | | | | | |
| infusion | | | | | | |

^{a}Even though the nominal p-value is ≤0.05, statistical significance cannot be claimed due to MDS-UPDRS Part II not being significant
PKG = Parkinson's KinetiGraph ® or Personal KinetiGraph ® (United States), lower value indicating less impairment

### Sensitivity Analysis for Primary and First Key Secondary Endpoint Full Analysis Set

Missing data in active-control Oral CD/LD group is assumed to be missing-at-random (MAR)
Missing data in foscarbidopa/foslevodopa subcutaneous infusion group is assumed to jump-to-reference (J2R) after foscarbidopa/foslevodopa subcutaneous infusion discontinuation
Analytic approach is used to estimate the standard error
The results from J2R sensitivity analysis support the results from the primary MMRM analysis

TABLE 9

| MMRM | | | J2R analytic approach | |
|---|---|---|---|---|
| | LS Mean of | | LS Mean of | |
| LS Mean | difference | p- | difference | p- |
| (SE) | (SE) | value | (SE) | value |

Primary: Change from Baseline to Week 12 in hours of average
daily normalized “On” time without troublesome dyskinesia

| | | | | | |
|---|---|---|---|---|---|
| Oral CD/LD | 0.97 (0.50) | | | | |
| foscarbidopa/foslevodopa | 2.72 (0.52) | 1.75 (0.65) | 0.0083 | 1.14 (0.44) | 0.0093 |
| subcutaneous infusion | | | | | |

Key Secondary #1: Change from Baseline to Week 12 in hours
of average daily normalized “Off” time

| | | | | | |
|---|---|---|---|---|---|
| Oral CD/LD | −0.96 (0.49) | | | | |
| foscarbidopa/foslevodopa | −2.75 (0.50) | −1.79 (0.63) | 0.0054 | −1.16 (0.42) | 0.0061 |
| subcutaneous infusion | | | | | |

### Labs, Vital Signs, ECG and C-SSRS Findings: Safety Analysis Set

No clinically meaningful changes or trends from baseline were observed based on the review of laboratory results, vital signs and ECG for either treatment group, and no safety concerns were identified.
There was no evidence of increased suicidality for either treatment group based on the review of C-SSRS data.

### Blinding Assessment

TABLE 10

| | | | foscarbidopa/foslevodopa | |
|---|---|---|---|---|
| | Oral CD/LD | | subcutaneous infusion | |
| | (N = 67) | | (N = 74) | |
| Number of subjects responded | 64 | | 70 | |
| Answer by subjects | | | | |
| foscarbidopa/foslevodopa | 10 | (15.6) | 52 | (74.3) |
| subcutaneous infusion | | | | |
| Not sure | 8 | (12.5) | 11 | (15.7) |
| Oral CD/LD | 46 | (71.9) | 7 | (10.0) |
| Certainty | | | | |
| Neutral | 2 | (3.1) | 2 | (2.9) |
| Somewhat certain | 12 | (18.8) | 14 | (20.0) |
| Somewhat uncertain | 5 | (7.8) | 4 | (5.7) |
| Very certain | 31 | (48.4) | 32 | (45.7) |
| Very uncertain | 5 | (7.8) | 6 | (8.6) |
| Reason (Each subject can be | | | | |
| counted multiple times) | | | | |
| Improvement of PD symptoms | 10 | (15.6) | 44 | (62.9) |
| Infusion liquid | 5 | (7.8) | 4 | (5.7) |
| No change of PD symptoms | 31 | (48.4) | 6 | (8.6) |
| Other | 3 | (4.7) | 9 | (12.9) |
| Side effect after infusion | 2 | (3.1) | 9 | (12.9) |
| Site staff | 1 | (1.6) | 0 | |
| Worsening of PD symptoms | 12 | (18.8) | 7 | (10.0) |

### Conclusions

Efficacy: treatment with foscarbidopa/foslevodopa subcutaneous infusion resulted in statistically significant and clinically meaningful improvements compared with oral CD/LD in
   the primary efficacy endpoint: “On” time without troublesome dyskinesia
   First key secondary endpoint: “Off” time

### Example 2. Safety, Tolerability, and Pharmacokinetics in Healthy Subjects

This example demonstrates that foslevodopa and foscarbidopa at a ratio of 20:1 is safe and also evaluates its pharmacokinetics.

### Methodology

To assess safety, tolerability, and pharmacokinetics of foslevodopa and foscarbidopa delivered as a single continuous subcutaneous infusion (CSCI) over 16 hours, a total of 8 healthy older (45-75-year-old) human subjects participated in a single-blind, placebo-controlled, 3 period cross-over design clinical study. Each dose of foslevodopa and foscarbidopa or placebo was administered in a single blind manner as a single CSCI to the abdomen delivered at a constant rate over a 16-hour period via an infusion set connected to an ambulatory pump. Regimens A, B, C, and D consisted of 640/160 mg levodopa 4′-monophosphate/carbidopa 4′-monophosphate, 640/64 mg levodopa 4′-monophosphate/carbidopa 4′-monophosphate CSCI, 640/32 mg levodopa 4′-monophosphate/carbidopa 4′-monophosphate CSCI, or placebo CSCI, respectively, delivered in the abdomen at a constant rate over 16 hours. Blood samples were collected for pharmacokinetic analysis prior to priming of catheter, prior to infusion (0 hour), and at 0.5, 1, 2, 4, 8, 12, 16, 20, 24, 26 and 28 hours after start of infusion from each of the of 8 healthy older (45-75-year-old) human subjects.

### Population

Qualified subjects were healthy male and female volunteers whose ages were between 45 and 75 years, inclusive. If female, subject must be postmenopausal for at least 1 year or surgically sterile. Exclusion criteria included a history of significant skin conditions or disorders (e.g., psoriasis, atopic dermatitis, etc.) or evidence of recent sunburn, acne, scar tissue, tattoo, open wound, branding, or colorations.

### Safety Results

No pattern was evident with regard to the nature or frequency of treatment-emergent adverse events following CSCI infusion or bolus injection of the levodopa 4′-monophosphate and carbidopa 4′-monophosphate composition compared to subjects who received placebo. All regimens tested were well tolerated by the subjects. No concerning patterns of adverse events or laboratory findings were reported. There were no notable observations on individual measurements for blood pressure or pulse rate, nor from quantitative measurements from the ECGs.

### Example 3. Continuous Subcutaneous Infusion of Levodopa and Carbidopa Prodrugs in Healthy Volunteers for 24 Hours: Safety and Tolerability Over 10 Days to Simulate 1 Year of Exposure

This study was designed to assess the safety and local tolerability of 24 hour continuous subcutaneous infusion of an aqueous pharmaceutical composition comprising foslevodopa and foscarbidopa. The study simulated one-year of exposure by administering the pharmaceutical composition for 10 consecutive days in a confined area of the abdomen of healthy volunteers.

### Methodology

The study was a phase 1, randomized, placebo-controlled, study of healthy volunteers receiving the pharmaceutical composition sufficient to provide 600 mg of levodopa and an equal volume of saline via 24-hour continuous subcutaneous infusion for 10 days simultaneously on opposite sides of the abdomen. The study consisted of 3 periods as shown in Table 11.

TABLE 11
Study Design Schematic of Safety and Tolerability
Study over 10 days to Simulate 1 year of Exposure

| | | | | | | | | | | | | | Fol- |
|---|---|---|---|---|---|---|---|---|---|---|---|---|---|
| | | | | | | | | | | | | | low- |
| | | | | | | | | | | | | | up |
| Screen- | | Confinement Period | | | | | | | | | | | (28 ± |
| ing (28 | | Co-administration of the Pharmaceutical | | | | | | | | | | | 3 |
| days) | | Composition and Placebo | | | | | | | | | | | days |
| | | ↓ | ↓ | ↓ | ↓ | ↓ | ↓ | ↓ | ↓ | ↓ | ↓ | | |
| DAY | −1 | 1 | 2 | 3 | 4 | 5 | 6 | 7 | 8 | 9 | 10 | 11 | 12 |
| | | ↑ | ↑ | ↑ | ↑ | ↑ | ↑ | ↑ | ↑ | ↑ | ↑ | | |

Middle row DAY indicates required confinement or clinical visit
Top and bottom row boxes indicate day with blinded infusion site evaluation
↓ 24-hour subcutaneous infusion of placebo to infusion site
↑ 24-hour subcutaneous infusion of the pharmaceutical composition to infusion site

As shown in Table 11, the screening period lasted for 28 days and was conducted to ensure patients met eligibility and criteria and also to collect medical history and baseline clinical assessments. The next period was a confinement period during which patients were confined to the study site for 13 days (Day −1 to Day 12). During the confinement period, the infusion was started at Day 1 where each patient was simultaneously administered the pharmaceutical composition and equal volumes of placebo (saline). This infusion was administered in two 5 cm diameter areas on opposite sides of the abdomen. Subjects and infusion site raters were blinded as to which treatment was administered on each side of the abdomen. Infusion sets were changed daily, and the catheter of each infusion set was placed within a 5-cm diameter area of the site used on the previous day. Infusion was continuous for 24 hours/day over a 10 days period in which subjects received a levodopa equivalent dose of about 600 to about 700 mg/day. Next, the follow up period of 28 days was conducted to allow ad hoc reporting of any adverse events. Infusion sets were changed and reapplied daily on the same skin surface.

### Infusion Site Rotation

Good clinical practice and anecdotal data recommended rotating injection sites regularly, keeping them at least 2.5 cm apart, to reduce risk of infection or irritation, fatty tissue build up (hypertrophy) and scar formation (fibrosis). The infusion set selected for this study (Smith Cleo 90) recommended changing the set every 3 days to preserve set sterility. In clinical practice, it is expected that patients will adopt a rotation scheme such as a clock that allows a rotation that allows using the same infusion site after 11 alternative sites have been used. Individuals adopt a rotation schedule for infusion sites around the navel (center). If rotation begins at the 12 o'clock position and proceeds clockwise, assuming that each infusion site is used of 3 days, patients will return to the infusion site at the 12 o'clock position after approximately 36 days −(12*3=) 36 days, with an average use of the same site of infusion 10 times/year. This study provided an accelerated simulation of longer-term use for assessment of local tolerability in healthy human volunteers for the number of repeat infusion sites that a patient would use over the course of a year.

### Population

The key inclusion criteria for the study were:
Adult male or female healthy human volunteers 45 to 75 years of age;
Body Mass Index (BMI) from 18.0 kg/m2 to 32.0 kg/m2, inclusive;
A condition of general good health, based upon medical history, physical examination, and no clinically significant laboratory values, Electrocardiogram (ECG), or vital parameters; and
No history of significant skin conditions or disorders the study investigator determines might interfere with study assessments.

### Assessments and Analysis

Systemic and local safety and tolerability were assessed daily. Notable skin reactions were defined apriorias events normally not associated with predictable reactions from the use of infusion sets (grades≥D or ≥6 on the Infusion Site Evaluation Scales). Both subjects and raters were blinded to the sides of the abdomen in which the aqueous pharmaceutical composition comprising the levodopa 4′-monophosphate and carbidopa 4′-monophosphate, and placebo were infused. Specifically, the local skin tolerability of 24-hours continuous subcutaneous infusion of the pharmaceutical composition in the abdomen for 10 days in 33 healthy human volunteers who completed the 10-day dosing period was assessed. Local skin tolerability was assessed by a blinded rater using the Infusion Site Evaluation 2-part scale (Table 12). This evaluation included numeric grading (0-7) and letter grading (A-G) scales. Notable skin reactions were defined a-priori as events normally not associated with predictable reactions from the use of infusion sets (grades≥D or ≥6). The primary endpoint was the number of healthy human volunteers who had a notable skin reaction at the pharmaceutical composition infusion site on >2 days of the 10-day infusion. A 95% upper confidence bound for the proportion of the population who would have a notable skin reaction on >2 days of a 10-day infusion was obtained by the Clopper-Pearson method. For each infusion site evaluation scale, a one-sided sign test was performed to test the hypothesis of no difference between the pharmaceutical composition and placebo at the final evaluation against the alternative hypothesis that the pharmaceutical composition is more likely to have a higher grade.

TABLE 12
Infusion Site Assessment Grading Scales

| Numeric Grading (Part 1) | | Letter Grading (Part 2) | |
|---|---|---|---|
| Grade | Description | Grade | Description |
| 0 | No evidence of irritation | A | No finding |
| 1 | Minimal erythema, barely perceptible | B | Slight glazed appearance |
| 2 | Moderate erythema, readily visible; or | C | Marked glazing |
| | minimal edema; or minimal papular | | |
| | response | | |
| 3 | Erythema and papules | D | Glazing with peeling and cracking |
| 4 | Definite erythema | E | Glazing with fissures |
| 5 | Erythema, edema, and papules | F | Film of dried serous exudates covering all |
| | | | or portion of the patch site |
| 6 | Vesicular eruption | G | Small petechial erosions and/or scabs |
| 7 | Strong reaction spreading beyond the test | | |
| | site | | |

Safety assessments included the following in addition to the infusion site evaluation: The percentage of subjects with treatment-emergent AEs and serious adverse events (SAEs); Change from baseline to end of study in clinical laboratory values, vital sign measurements, electrocardiograms (ECGs), and physical examination findings.

### Results

The safety dataset included 34 subjects as presented in Table 13.

TABLE 13
Baseline demographics

| Characteristic | Safety Population (N = 34) | |
|---|---|---|
| Gender, n (%) | | |
| Female | 9 | (26.5) |
| Male | 25 | (73.5) |
| Ethnicity, n (%) | | |
| Hispanic or Latino | 5 | (14.7) |
| Not Hispanic or Latino | 29 | (85.3) |
| Race, n (%) | | |
| White | 24 | (70.6) |
| Black or African American | 7 | (20.6) |
| Multiple | 3 | (8.8) |
| Age, years, | | |
| mean (SD) | 56.1 | (7.2) |
| range | 45-69 | |
| Weight (kg), mean (SD) | 81.6 | (10.1) |
| BMI (kg/m²), mean (SD) | 27.0 | (2.7) |

BMI = body mass index; SD = standard deviation.

### Tolerability

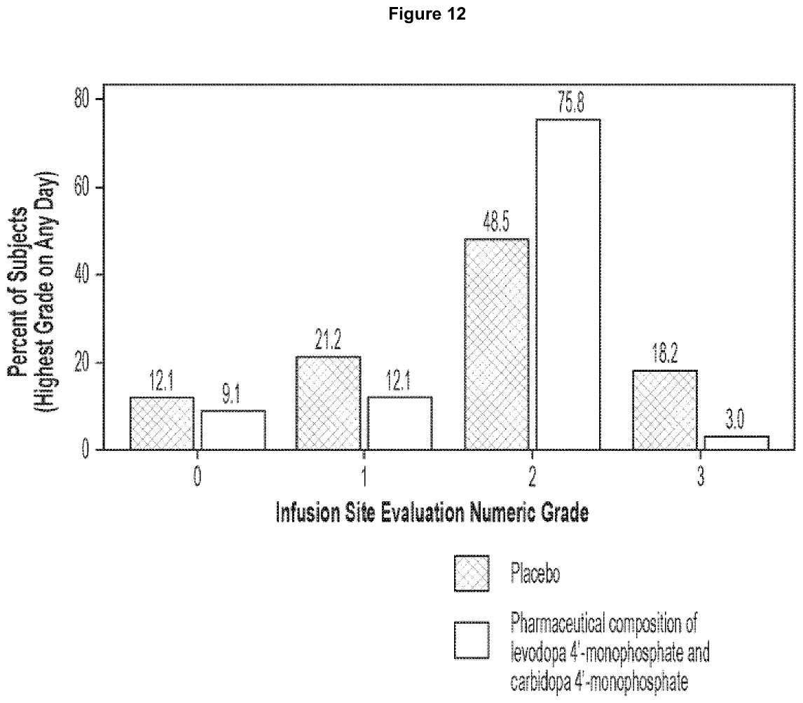

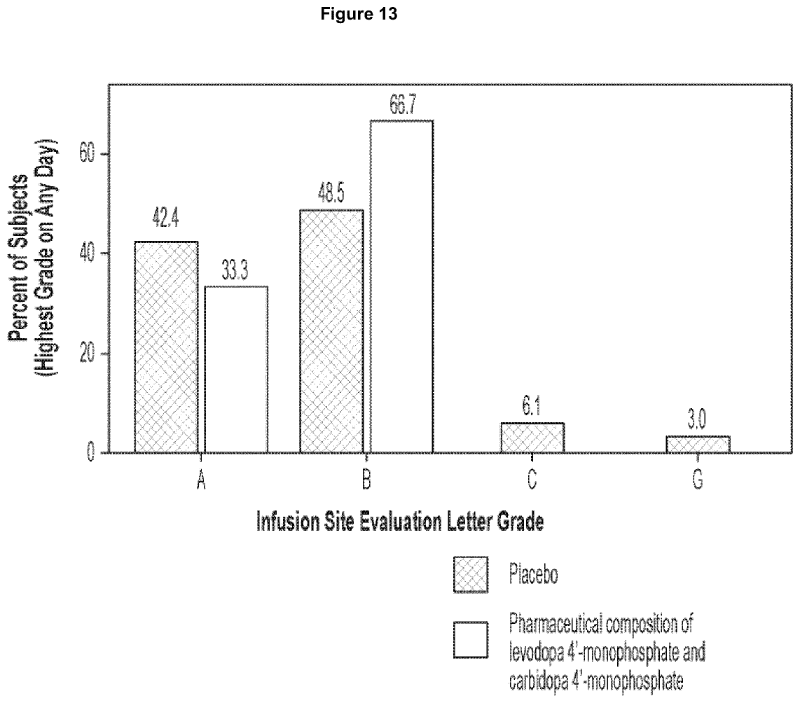

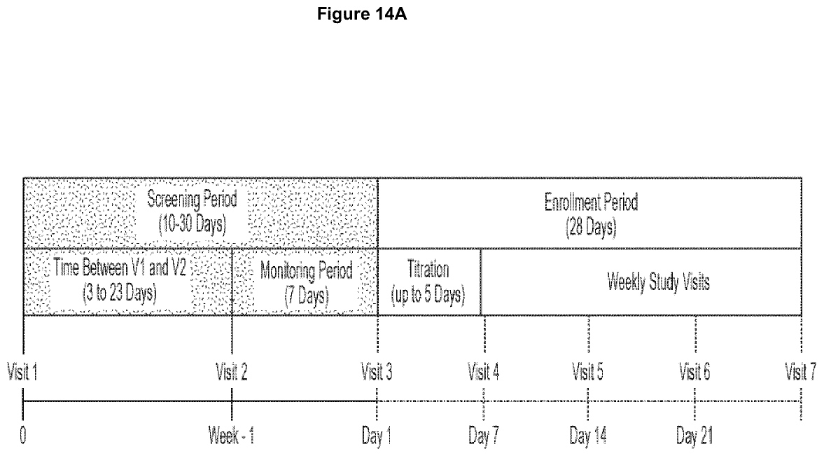

The highest grade reported for each healthy human volunteer subject (the pharmaceutical composition vs placebo sites) from Day 1 to Day 10 of the study is summarized graphically for the respective infusion site assessment scales in FIG. 12 and FIG. 13. Out of the subjects who completed the 10-day dosing, the percent of subjects who reported notable skin reactions was 0% (0/33) at the pharmaceutical composition infusion site and 3.0% (1/33) at the placebo infusion site. The difference between the pharmaceutical composition and placebo with respect to dermatologic assessment on Day 10 of infusion was not statistically significant for the numeric (P=0.828) or letter (P=0.363) grading scales. The 95% upper confidence bound for the proportion of the population that would have a notable skin reaction on more than 2 days of a 10-day infusion of the pharmaceutical composition as administered in this study is 0.087 (8.7% of the population). The 95% upper confidence bound for the proportion of the population that would have a notable skin reaction on more than 2 days of a 10-day placebo infusion as administered in this study is also 0.087 (8.7% of the population).

### Safety

There were no clinically significant laboratory values, vital signs, or ECG findings. Overall, 97% of subjects reported at least one adverse event (AE). There was one serious AE reported 4 days after study completion, which was considered not potentially related to foslevodopa and foscarbidopa. There were no discontinuations due to an AE (Table 14). The most frequently reported adverse events were infusion site erythema (91%), infusion site reaction (44%), and infusion site pain (32%) (Table 15). All infusion site AEs were mild or moderate in severity and resolved quickly.

TABLE 14
Treatment- Emergent Adverse Events
in Healthy Human Volunteer Subjects

| | Overall | |
|---|---|---|
| | N = 34 | |
| Subjects with Any Adverse Events | N (%) | |
| Adverse Event | 33 | (97.1) |
| AE with reasonable possibility | 17 | (50.0) |
| of being drug-related | | |
| Severe AE | 1 | (2.9)* |
| Serious AE | 1 | (2.9)* |

| | |
|---|---|
| AE leading to discontinuation | 0 |
| of foslevodopa and foscarbidopa | |

*Musculoskeletal pain judged as not potentially related to foslevodopa and foscarbidopa.
AE = adverse event

TABLE 15
Infusion Site Treatment-Emergent Adverse Events
in Healthy Human Volunteer Subjects

| | Overall | | Prodrug Combination | | Placebo | |
|---|---|---|---|---|---|---|
| | N = 34 | | N = 34 | | N = 34 | |
| | n (%) | | n (%) | | n (%) | |
| Infusion site AEs | 31 | (91.2) | 30 | (88.2) | 30 | (88.2) |
| of interest | | | | | | |
| Bruising | 4 | (11.8) | 2 | (5.9) | 2 | (5.9) |
| Erosion | 1 | (2.9) | 0 | | 1 | (2.9) |
| Erythema | 31 | (91.2) | 28 | (82.4) | 30 | (88.2) |
| Hemorrhage | 1 | (2.9) | 1 | (2.9) | 1 | (2.9) |
| Edema | 9 | (26.5) | 9 | (26.5) | 0 | |
| Pain | 11 | (32.4) | 11 | (32.4) | 2 | (5.9) |
| Reaction | 15 | (44.1) | 14 | (41.2) | 13 | (38.2) |

The pharmaceutical composition described herein comprising foslevodopa and foscarbidopa provides the broad range of levodopa exposure required to adequately control motor symptoms and to be an alternative therapeutic option for Parkinson's disease patients. This study demonstrated that the pharmaceutical composition was generally well tolerated and did not cause notable skin reactions at low, yet clinically relevant, doses administered subcutaneously in a confined area of the abdomen continuously for 10 consecutive days.

### Example 4. Design for a Phase 1b Study Evaluating the Safety and Tolerability of a 4-Week Continuous Subcutaneous Infusion of Levodopa and Carbidopa Prodrugs in Parkinson's Disease Patients

This study was designed to evaluate the safety and tolerability of a 4-week continuous subcutaneous infusion of an aqueous pharmaceutical composition foslevodopa and foscarbidopa in a w/w 20:1 ratio. In addition, the steady-state plasma levodopa levels achieved by the continuous subcutaneous infusion of the pharmaceutical composition were assessed and the exploratory efficacy were evaluated by the change from baseline in the endpoints listed in Table 21A.

### Method

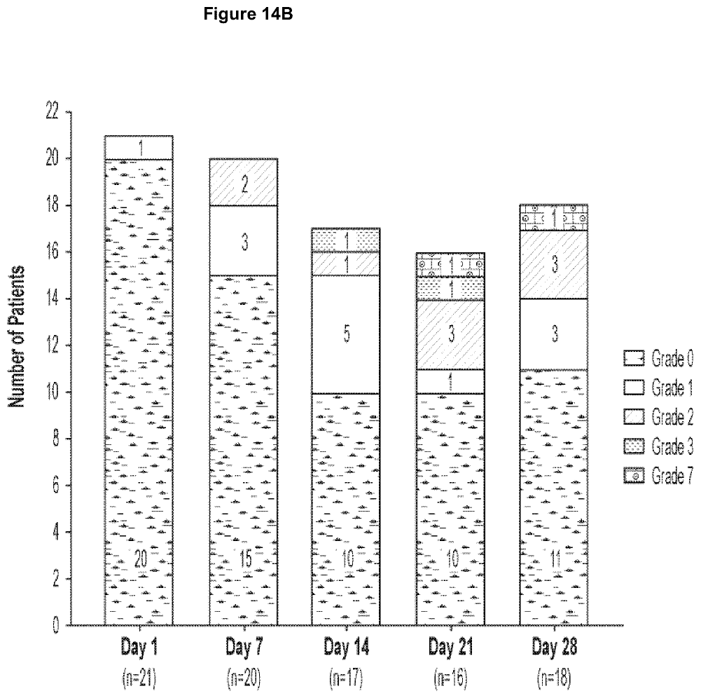

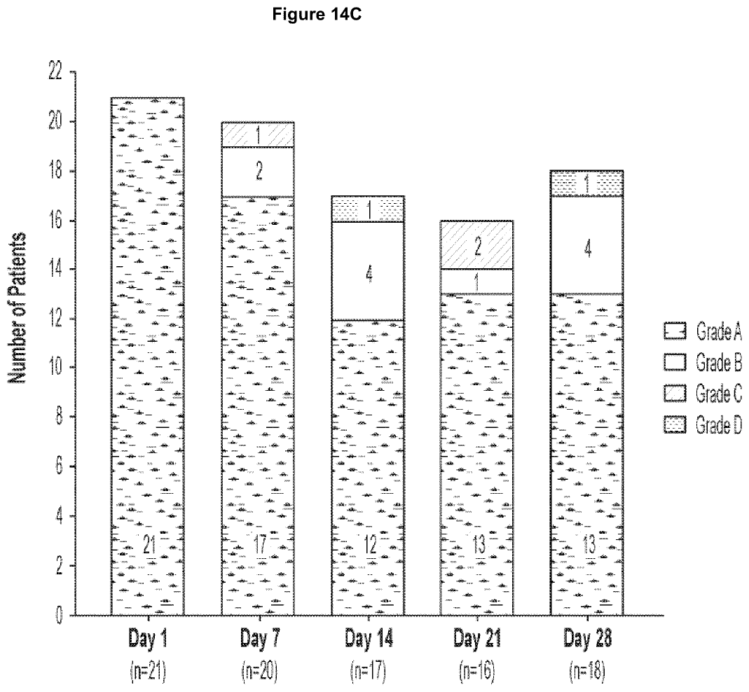

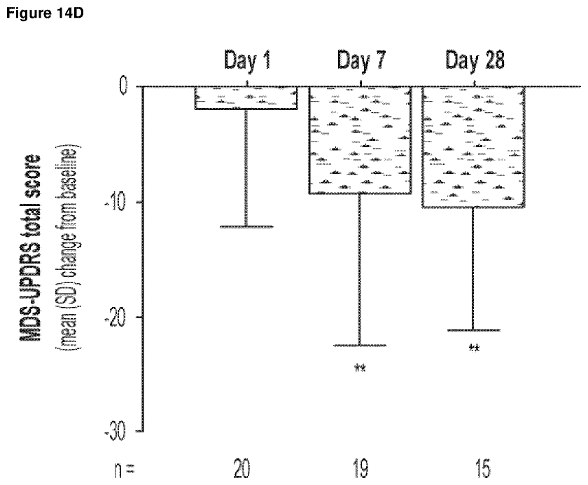

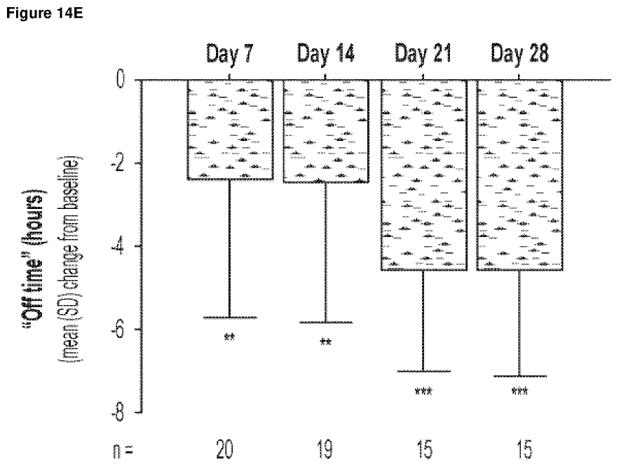

A single-arm, open-label, phase 1b study of patients with Parkinson's disease treated with personalized therapeutic doses of the pharmaceutical composition via 24-hour continuous subcutaneous infusion for 28 days was designed. Patients were recruited from sites in the United States. The study consisted of 4 periods and is graphically represented in FIG. 14A. The Screening Period included 2 visits including the monitoring period to establish eligibility and to confirm that the patient's current Parkinson's disease therapy has been stable for 30 days. The Screening Period also included a Monitoring period for the 7 days immediately following visit 2. Patients recorded Parkinson's disease medications using a subject dosing diary and monitored motor symptoms using a wearable motion sensor device configured to detect abnormal movements.

The titration period was part of the Enrollment Period and followed the Screening Period. On day 1 of the titration period, patients received a bolus dose of the pharmaceutical composition followed by a continuous infusion at a constant rate, with subsequent dose adjustments at the investigator's discretion based on the patient's clinical response. The therapeutic dose is defined as the dose able to elicit an adequate control of motor symptoms by minimizing the number of “Off” episodes and maximizing the functional “On” time while minimizing troublesome dyskinesia. Patients continued receiving the therapeutic dose of the pharmaceutical composition established during the titration period until day 28—the Treatment period. The key inclusion criteria for the study are summarized in Table 16.

TABLE 16
Key Inclusion and Exclusion Criteria in Patients

| Inclusion | Exclusion |
|---|---|
| Adult male or female patients 30 | Previous exposure to the |
| to 85 years of age with a clinical | pharmaceutical |
| diagnosis of levodopa-responsive | composition |
| idiopathic Parkinson's disease | |
| Patients whose symptoms are judged | History of significant skin |
| by the investigator to be inadequately | conditions or disorders the study |
| controlled by current stable therapy | investigator determines might |
| | interfere with study assessments |
| Have a recognizable/identifiable | MMSE score <24 |
| “Off” and “On” state | |
| Have a minimum of 2.5 hours | Abnormal laboratory values, ECG, |
| of “Off” time/day | or vital parameters at screening |

ECG = electrocardiogram; MMSE = mini-mental state examination; PD = Parkinson's disease.

Systemic safety and tolerability were assessed by adverse event monitoring, laboratory values, vital signs, electrocardiogram, and safety scales, including the Columbia-Suicide Rating Scale. The infusion site grading scales (Table 17A) and the exploratory efficacy assessments (Table 17B) were used to assess the outcome of the study.

Subjects recorded Parkinsonian symptoms based on the questionnaire in the Parkinson's Disease Diary. Each subject recorded whether he/she was “On”, “Off”, or “Asleep” and the severity of his/her dyskinesias (troublesome or not troublesome). Statistical significance for change from baseline was shown at each visit for normalized “Off” time, normalized “On” time without dyskinesia, and normalized “On” time without troublesome dyskinesia. Statistical significance was not shown at any visit for normalized “On” time with non-troublesome dyskinesia and normalized “On” time with troublesome dyskinesia.

Evaluation of the subject MDS-UPDRS consisted of the following sections:
Part 1: Non-Motor Aspects of Experiences of Daily Living (nM-EDL)
Part 2: Motor Aspects of Experiences of Daily Living (M-EDL)
Part 3: Motor Examination (including Hoehn and Yahr stage)
Part 4: Motor Complications

The MDS-UPDRS Total Score ranges from 0 to 176, with 176 representing the worst (total) disability and 0 as no disability. Mean Total Baseline Scores ranged from approximately 45 to 47 for all visits and Mean Visit Total Scores ranged from approximately 34 to 45 for all visits. Statistically significant changes from baseline were shown on Day 7, Day 28, and Final Visit for Total Score, Part 1 Score, and Part 2 Score, and on Day 28 and Final Visit for Part 4 Score. There was no statistically significant change at any visit for Part 3 “On” Score.

The PDQ-39 measured aspects of health that are relevant to subjects with Parkinson's Disease. Each item was scored on the following 5-point scale: 0=never, 1=occasionally, 2=sometimes, 3=often, 4=always (or cannot do at all, when applicable). Higher scores are consistently associated with more severe symptoms of the disease such as tremors and stiffness. The majority of subjects responded with “never” or “occasionally”, while seven subjects responded with “always” or “cannot do at all.” The results are presented as a summary index. The PDQ-39 summary index ranged from 0 to 100 where lower scores indicated a better perceived health status. The domains and indices used for evaluation are as follows:

Summary Index
Mobility Domain Score
Activities of Daily Living Domain
Emotional Well-being Domain Score
Stigma Domain Score
Social Support Domain Score
Cognition Domain Score
Communication Domain Score
Bodily Discomfort Domain Score

Statistically significant changes from baseline were shown for all visits for the Summary Index scores.

The PDSS-2 scale characterizes the various aspects of nocturnal sleep problems in subjects with Parkinson's disease. The PDSS-2 consisted of 15 questions that evaluated motor and non-motor symptoms at night and upon wakening, as well as disturbed sleep grouped into 3 domains: motor symptoms at night, PD symptoms at night, and disturbed sleep. Scores were calculated for each domain as well as a total score. The frequency was assessed for the sleep problems based on a 5-point Likert-type scale ranging from 0 (never) to 4 (very often). The majority of subjects responded with “never” or “occasionally”, while seven subjects responded with “always” or “cannot do at all.”

The KPPS assessed pain among subjects with Parkinson's disease. The scale measured the frequency and severity of seven domains of pain: musculoskeletal, chronic, fluctuation related, nocturnal, orofacial, local limb pain/edema/swelling, and radicular pain. The Total Score was also assessed. Statistically significant changes from baseline were shown for the Total Score on Day 28 and at the Final Visit, and for Fluctuated Related Pain Score on Day 28.

The PAS measured the severity of anxiety in subjects with Parkinson's disease. Scores for persistent anxiety, episodic anxiety, and avoidance behavior, as well as Total Score were assessed. Statistically significant changes from baseline were shown for Day 28 and Final Visit for Total Score and Avoidance Behavior.

All subjects wore a Kinesia 360 device that continuously recorded data for the assessment of tremor, dyskinesia, and mobility. There was no statistically significant change from baseline at any visit for tremor, dyskinesia, and slowness.

Infusion site assessment grading scales and exploratory efficacy assessments conducted and shown in Tables 17A and 17B below.

TABLE 17A
Infusion Site Assessment Grading Scales

| | |
|---|---|
| Grade | Description |

Numeric Grading (Part 1)

| | |
|---|---|
| 0 | No evidence of irritation |
| 1 | Minimal erythema, barely perceptible |
| 2 | Moderate erythema, readily |
| | visible; or minimal edema; |
| | or minimal papular |
| | response |
| 3 | Erythema and papules |
| 4 | Definite erythema |
| 5 | Erythema, edema, and papules |
| 6 | Vesicular eruption |
| 7 | Strong reaction spreading |
| | beyond the test site |

Letter Grading (Part 2)

| | |
|---|---|
| A | No finding |
| B | Slight glazed appearance |
| C | Marked glazing |
| D | Glazing with peeling and cracking |
| E | Glazing with fissures |
| F | Film of dried serious exudates |
| | covering all or portion of the |
| | patch side |
| G | Small petechial erosions |
| | and/or scabs |

TABLE 17B
Exploratory Efficacy Assessments

| Endpoint | Assessment |
|---|---|
| Average daily “Off” | Parkinson's Disease diaries |
| and “On” times | |
| Parkinson's Disease | Movement Disorder Society-Unified Parkinson's |
| symptoms | Disease Rating Scale (UPDRS) |
| Motor symptoms | Kinesia 360 wearable device |
| Sleep symptoms | Parkinson's Disease Sleep Scale-2 (PDSS-2) |
| Quality of life | Parkinson's Disease Questionnaire-39 item |
| | (PDQ-39) |
| Anxiety symptoms | Parkinson's Anxiety Scale (PAS) |
| Pain symptoms | King's Parkinson's Disease Pain Scale (KPPS) |

Exploratory analyses were conducted to assess the efficacy of the pharmaceutical composition on Parkinson's disease symptoms in reducing “Off” time as well as motor and non-motor symptoms.

### Results

Twenty-one patients were enrolled. The study population was primarily male (61.9%) and White (100%); 1 (4.8%) subject was Hispanic or Latino. Mean (SD) age was 61.6 (10.3) years. The mean (SD) Parkinson's disease duration since diagnosis was 9.0 (4.0) years and the mean (SD) duration of motor fluctuation was 6.0 (4.1) years. The average “Off” time/day at baseline was 6.54 hours, ranging from 3.77 to 9.46 hours. Seven subjects (33%) prematurely discontinued. Two subjects discontinued due to adverse events.

Baseline demographics and disease characteristics of the subjects are shown in Table 18.

TABLE 18
Baseline Demographics and Disease Characteristics in Patients

| | | | Overall | |
|---|---|---|---|---|
| | | | N = 21 | |
| Gender, n (%) | Female | | 8 | (38) |
| | Male | | 13 | (62) |
| Race, n (%) | White | | 21 | (100) |
| Age, years, mean (SD) | | | 61.6 | (10.3) |
| Parkinson's Disease duration, | <10 | years | 12 | (57.1) |
| year, n (%) | ≥10 | years | 9 | (42.9) |

| | | |
|---|---|---|
| Age at onset of Parkinson's Disease, | 52.0 | (10.6) |
| years, mean (SD) | | |
| Time since onset of Parkinson's Disease, | 10.1 | (4.0) |
| years, mean (SD) | | |
| Age at Parkinson's Disease diagnosis, | 53.2 | (10.9) |
| years, mean (SD) | | |
| Time since Parkinson's Disease diagnosis, | 9.0 | (4.0) |
| years, mean (SD) | | |
| Age at onset of motor fluctuations, | 56.2 | (10.5) |
| years, mean (SD) | | |
| Time since onset of motor fluctuations, | 6.0 | (4.1) |
| years, mean (SD) | | |
| MMSE total score*, mean (SD) | 28.8 | (1.4) |

*Patient must have an MMSE score ≥24 for inclusion in the study. MMSE = Mini-Mental State Examination; SD = standard deviation.

The results of the infusion site grading are shown in FIGS. 14B and 14C and Table 19, below.

TABLE 19
Infusion Site Treatment-Emergent
Adverse Events (AEs) in Patients

| | Mild | | Moderate | Severe | Total | |
|---|---|---|---|---|---|---|
| | n (%) | | n (%) | (n %) | n (%) | |
| Infusion site AEs | 12 | (57.1%) | 1 (4.8%) | 1 (4.8%) | 14 | (66.7%) |
| of interest | | | | | | |
| Discomfort | 1 | (4.8%) | 0 | 0 | 1 | (4.8%) |
| Erythema | 5 | (23.8%) | 1 (4.8%) | 0 | 6 | (28.6%) |
| Irritation | 2 | (9.5%) | 1 (4.8%) | 0 | 3 | (14.3%) |
| Nodule | 2 | (9.5%) | 0 | 0 | 2 | (9.5%) |
| Pain | 6 | (28.6%) | 1 (4.8%) | 0 | 7 | (33.3%) |
| Pallor | 1 | (4.8%) | 0 | 0 | 1 | (4.8%) |
| Papule | 0 | | 1 (4.8%) | 0 | 1 | (4.8%) |
| Rash | 1 | (4.8%) | 0 | 0 | 1 | (4.8%) |

| | | | | | |
|---|---|---|---|---|---|
| Reaction | 0 | 0 | 1 (4.8%) | 1 | (4.8%) |
| Warmth | 0 | 1 (4.8%) | 0 | 1 | (4.8%) |

Table depicts the most severe AE for each preferred term as assessed for each patient; patients are counted once in each row; therefore, the sum is greater than the total in each column. AE = adverse event.

In Parkinson's disease “Off”-time refers to periods of the day when the medications are not working well, causing reappearance or worsening of parkinsonian symptoms (tremor, rigidity, bradykinesia, as well as non-motor symptoms such as depression, pain, anxiety). In contrast, the term “On”-time refers to periods of adequate control of symptoms. “Off”-time can sometimes occur predictably and gradually (“wearing off”), or it may emerge suddenly and unexpectedly (“sudden Off”, “yo-yo episodes”). The frequency and timing of wearing-off periods and the number of hours in “Off” time significantly correlate with a worsening in quality of life for Parkinson's patients.

Quality of life in Parkinson's disease can be assessed using tools and questionnaires, such as the Parkinson's Disease Questionnaire-39 items (PDQ-39), a self-report questionnaire which assesses the impact of Parkinson's disease on specific dimensions of functioning and well-being, and the PDSS-2, a revised version of the Parkinson's Disease Sleep Scale, designed to characterize and quantify the various aspects of nocturnal sleep problems in Parkinson's disease. The disease severity was instead evaluated via the Unified Parkinson's Disease Rating Scale (UPDRS) or via the Movement Disorders Society revised version (MDS-UPDRS), a tool comprised of a rater-based interview and clinical assessment designed to provide a quantifiable score for longitudinal assessment and follow-up of the disease.

Patient changes from baseline normalized “Off” time were measured as shown in Table 20 and shown in FIG. 14E. A summary of pre-specified efficacy endpoints is provided in Table 21A and efficacy assessments are shown in Table 21B.

TABLE 20
Patient changes from baseline normalized “Off” time

| | Average | Average | | |
|---|---|---|---|---|
| | Normalized “Off” | Normalized “Off” | Improve- | Percent |
| | Time (hours) at | Time (hours) at | ment | Improve- |
| Subject | Baseline^{a} | End of Study^{b} | (hours) | ment |
| 17-1 | 6.51 | 2.64 | 3.87 | 59.40 |
| 17-2 | 7.25 | 4.63 | 2.62 | 36.17 |
| 17-3 | 4.77 | 0.18 | 4.59 | 96.27 |
| 17-4^{c,e} | 4.87 | 11.22 | −6.36^{d} | −130.64^{d} |
| 17-5 | 4.85 | 5.01 | −0.17^{d} | −3.41^{d} |
| 17-6 | 8.89 | 0.37 | 8.51 | 95.79 |
| 17-7 | 9.46 | 0.60 | 8.86 | 93.70 |
| 17-8^{c} | 8.67 | 8.01 | 0.66 | 7.60 |
| 17-9° | 7.93 | 4.00 | 3.93 | 49.59 |
| 17-10^{c,f} | 5.80 | 9.41 | −3.61^{d} | −62.25^{d} |
| 17-11 | 7.33 | 2.51 | 4.82 | 65.79 |
| 17-12 | 6.91 | 1.19 | 5.72 | 82.77 |
| 17-13^{c} | 5.96 | 4.29 | 1.67 | 27.98 |
| 17-14 | 8.80 | 1.45 | 7.35 | 83.48 |
| 17-15 | 6.18 | 2.47 | 3.71 | 60.07 |
| 17-16 | 6.37 | 3.62 | 2.75 | 43.15 |
| 17-17 | 3.99 | 0 | 3.99 | 100 |
| 17-18 | 3.77 | 1.16 | 2.61 | 69.18 |
| 17-19 | 5.76 | 0.59 | 5.16 | 89.71 |
| 17-20^{c} | 6.81 | 2.80 | 4.02 | 58.95 |
| Average | 6.54 | 3.31 | 3.24 | 46.17 |

^{a}Average baseline “Off” time is calculated as the average “Off” time from Days −3 through Day −1.
^{b}Average “Off” time at the end of the study is calculated as the average “Off” time prior to the last dose of foslevodopa and foscarbidopa.
^{c}Prematurely discontinued foslevodopa and foscarbidopa.
^{d}Worsening in normalized “Off” time.
^{e}Subject not compliant with infusion set procedures.
^{f}Subject discontinued the foslevodopa and foscarbidopa but agreed to complete study visits.

TABLE 21A
Pre-specified efficacy endpoints in Patients

| | | | | | | Change from | |
|---|---|---|---|---|---|---|---|
| | | | | | | Baseline to | |
| | | Baseline | | Final | | Final | |
| Parameters | N | Mean (SD) | | Mean (SD) | | Mean (SD) | |
| Normalized “Off” time | 20 | 6.54 | (1.65) | 3.31 | (3.14) | −3.24 | (3.65) |
| (hours/day) | | | | | | | |
| Normalized “On” time without | 20 | 8.57 | (2.24) | 11.86 | (4.34) | 3.30 | (4.11) |
| troublesome dyskinesia^{a} | | | | | | | |
| (hours/day) | | | | | | | |
| Normalized “On” time without | 20 | 5.82 | (3.29) | 9.81 | (5.90) | 3.99 | (5.37) |
| dyskinesia (hours/day) | | | | | | | |
| Normalized “On” time with non- | 20 | 2.75 | (3.18) | 2.06 | (3.62) | −0.69 | (3.77) |
| troublesome dyskinesia | | | | | | | |
| (hours/day) | | | | | | | |
| Normalized “On” time with | 20 | 0.89 | (1.92) | 0.83 | (2.69) | −0.06 | (3.05) |
| troublesome dyskinesia | | | | | | | |
| (hours/day) | | | | | | | |
| MDS-UPDRS Total Score (Parts | 20 | 46.7 | (18.73) | 37.7 | (18.01) | −9.0 | (11.87) |
| I-III) | | | | | | | |
| PDQ-39 Summary Index | 20 | 23.2 | (8.99) | 16.3 | (9.16) | −6.9 | (8.39) |
| PDSS-2 Total Score | 20 | 22.7 | (14.25) | 20.7 | (17.67) | −2.0 | (11.48) |
| KPPS | 18 | 17.7 | (21.20) | 11.7 | (13.78) | −6.0 | (9.82) |
| PAS | 18 | 11.0 | (5.37) | 8.1 | (4.54) | −2.9 | (5.39) |
| Kinesia 360 Daily Tremor Score | 21 | 0.060 | (0.0984) | 0.075 | (0.1993) | 0.015 | (0.1674) |
| Kinesia 360 Daily Dyskinesia | 21 | 0.709 | (0.2652 | 0.682 | (0.2845) | −0.027 | (0.1932) |
| Score | | | | | | | |
| Kinesia 360 Daily Slowness | 17 | 1.438 | (0.5213) | 1.442 | (0.5392) | 0.004 | (0.3392) |

Score

TABLE 21B
Efficacy Assessments

| Assessment | Tool |
|---|---|
| Normalized “Off” and “On” time | PD Diaries |
| PD Symptoms | MDS-UPDRS Parts I-IV* |
| Sleep Symptoms | PDSS-2 |
| Quality of Life | PDQ-39 |
| Health-related Quality of Life | EQ-5D-5L |

*Or the UPDRS Parts I-V where a validated translation of the MDS-UPDRS is not available. EQ-5D-5L = EuroQoL 5 dimensions questionnaire; MDS-UPDRS = Movement Disorder Society-Unified Parkinson's Disease Rating Scale; PD = Parkinson's disease; PDQ-39 = 39-item PD Questionnaire; PDSS-2 = PD Sleepiness Scale-2.

Analysis of efficacy data showed that 17 of the 20 patients had improvement in “Off” time. The mean (SD) reduction of “Off” time from baseline to study end was 3.24 (3.65) hours across subjects (46.2% improvement). Four subjects reported>90% reduction of daily “Off” time at the end of the study. There was mean reduction in normalized “Off” time, “On” time with non-troublesome dyskinesia, and “On” time with troublesome dyskinesia which resulted in a mean (SD) improvement in normalized “On” time without dyskinesia of 3.99 (5.37) hours. As shown in FIG. 14D, the mean (SD) reduction from baseline was 9.0 (11.87) for Movement Disorder Society-Unified Parkinson's Disease Rating Scale (MDS-UPDRS) total score, 6.9 (8.39) for Parkinson's Disease Questionnaire-39 items (PDQ-39) summary index, and 2.0 (11.48) for Parkinson's Disease Sleep Scale-2 (PDSS-2) total score. Following continuous subcutaneous infusion of the pharmaceutical composition, statistically significant changes from baseline were observed for: Parkinson's Disease “On” and “Off” times for dyskinesia, MDS-UPDRS Total Score, Non-Motor Aspects of Experiences of Daily Living, Motor Aspects of Experiences of Daily Living, and Motor Complications, and PDQ-39 Summary Index, and domains for Activities of Daily Living, Emotional Well-being, Cognition, Communication, Mobility, Stigma, and Bodily Discomfort.

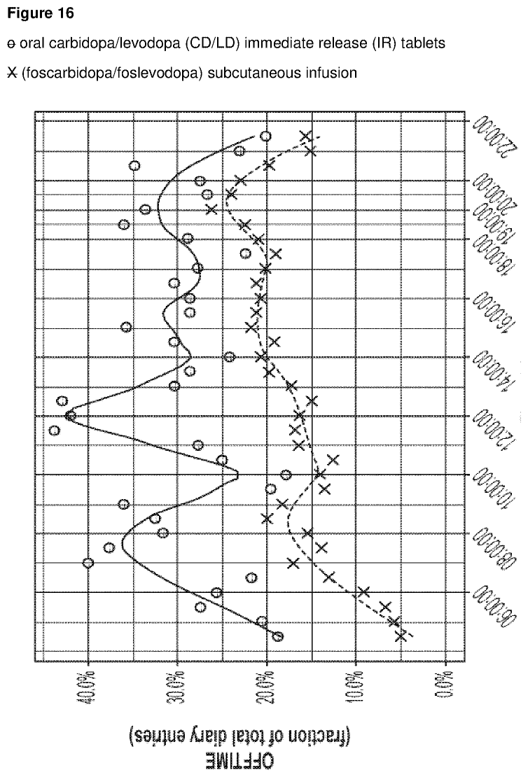

FIG. 16 and Table 21C show the percent of patients experiencing “Off” time receiving the pharmaceutical composition compared with patients receiving oral Sinemet®. As shown in FIG. 16, patients receiving oral Sinemet® treatment show undesirable spikes in “off” time in the morning and around meal times compared with patients receiving the CSCI pharmaceutical composition. Table 21C shows about 86.7% of the time the first morning symptom upon waking up was “Off” at baseline for patients receiving oral Sinemet®. Whereas, about 84.2% of the time the first morning symptom was “On” without dyskinesia at the end of the 28-day study for patients receiving the CSCI pharmaceutical composition.

TABLE 21C
Efficacy Assessments Early Morning First Non-
Sleep Symptom in Parkinson's disease Diary

| | | | | | | | On With Non- | | On With | |
|---|---|---|---|---|---|---|---|---|---|---|
| Non-Sleep | | | | | On Without | | Troublesome | | Troublesome | |
| Symptom | | | OFF | | Dyskinesia | | Dyskinesia | | Dyskinesia | |
| Visit | Day* | N | n | (%) | n | (%) | n | (%) | n | (%) |
| Baseline | 1 | 20 | 18 | (90.0) | 1 | (5.0) | 0 | | 1 | (5.0) |
| | 2 | 20 | 18 | (90.0) | 1 | (5.0) | 0 | | 1 | (5.0) |
| | 3 | 20 | 16 | (80.0) | 4 | (20.0) | 0 | | 0 | |
| | Average # | | | 86.7 | | 10.0 | | | | 3.3 |
| Day 7 | 1 | 20 | 4 | (20.0) | 14 | (70.0) | 1 | (5.0) | 1 | (5.0) |
| | 2 | 17 | 2 | (11.8) | 10 | (58.8) | 5 | (29.4) | 0 | |
| | 3 | 18 | 5 | (27.8) | 8 | (44.4) | 5 | (27.8) | 0 | |
| | Average # | | | 19.9 | | 57.7 | | 20.7 | | 1.7 |
| Day 14 | 1 | 15 | 3 | (20.0) | 12 | (80.0) | 0 | | 0 | |
| | 2 | 15 | 3 | (20.0) | 10 | (66.7) | 2 | (13.3) | 0 | |
| | 3 | 16 | 3 | (18.8) | 13 | (81.3) | 0 | | 0 | |
| | Average # | | | 19.6 | | 76.0 | | 4.4 | | |
| Day 21 | 1 | 15 | 1 | (6.7 | 14 | (93.3) | 0 | | | 0 |
| | 2 | 15 | 1 | (6.7) | 14 | (93.3) | 0 | | | 0 |
| | 3 | 13 | 2 | (15.4) | 10 | (76.9) | 1 | (7.7) | | 0 |
| | Average # | | | 9.6 | | 87.8 | | 2.6 | | |
| Day 28 | 1 | 13 | 0 | | 12 | (92.) | 1 | (7.7) | 0 | |
| | 2 | 14 | 2 | (14.3) | 11 | (78.6) | 1 | (7.1) | 0 | |
| | 3 | 11 | 2 | (18.2) | 9 | (81.8) | 0 | | 0 | |

| | | | |
|---|---|---|---|
| Average # | 10.8 | 84.2 | 4.9 |

Baseline is the last non-missing value prior to the beginning of the treatment period.
*: Day is days prior to visit date. If Day is X, PD diary date is X day(s) prior to visit date, XX = 1, 2, 3.
#: Average is the average percentage of 3 days.

### Example 5 Determining Starting Dose

To facilitate physicians' determination of the most appropriate starting dose for the study described in Example 4, an algorithm to convert oral levodopa to the carbidopa 4′-monophosphate and levodopa 4′-monophosphate pharmaceutical composition was created. This algorithm takes into consideration the low variability and fluctuation of levodopa exposures when delivered continuously subcutaneously, the 24-hour exposure, the pharmacokinetic profile of levodopa from previous studies with the composition, and other clinical considerations.

Data from the patients who completed dosing were used in the evaluation of dose. All patients who completed the study (N=14) achieved desired dose range within 3 weeks after starting the pharmaceutical composition; 3 patients achieved desired a dose range on the same day after oral-to-the pharmaceutical composition conversion, 7 subjects required 1 week, 1 subject required 2 weeks, while the remaining 3 patients required 3 weeks. Adjustments at each time ranged from −0.04 mL/h to +0.08 mL/h (the equivalent of −136 mg levodopa/day to 273 mg levodopa/day) while the difference in infusion rates from Day 1 to Day 28 (all adjustments considered) ranged from −0.06 mL/h to +0.08 mL/h (the equivalent of −204 mg levodopa/day to +273 mg levodopa/day). Considering that at the time of enrollment all patient subjects were required to report motor fluctuations that were inadequately controlled by their best oral medications, these data suggest that the conversion algorithm is effective in guiding the starting dose of the pharmaceutical composition because the magnitude of change in the continuous infusion rate was considered small (within 20% for all but 1 subject) and the desired dose range was achieved within 3 weeks.

Levodopa dose levels by patient subject at the start and at the end of the study are provided in Table 22. The doses of the pharmaceutical composition delivered in a 24-hour treatment period ranged from approximately 28.8/576 mg to about 240/4800 mg of carbidopa 4′-monophosphate/levodopa 4′-monophosphate per day (equivalent to approximately 400 mg to 3400 mg of levodopa respectively, based on molecular weight). The average and median doses at study end were approximately 117.5/2350 mg and 96/1920 mg (equivalent to 1670 mg of and 1360 mg of levodopa, respectively, based on molecular weight).

TABLE 22
By-Patient Subject Listing of levodopa Dose Levels
(mg/24 hours) at Study Start and Study End

| | Day 1 | | End of study | | Difference |
|---|---|---|---|---|---|
| | Levodopa 4′- | Levodopa | Levodopa 4′- | Levodopa | between end |
| | monophosphate | equivalents | monophosphate | equivalents | of study and |
| Subject | (mg/24 h) | (mg/24 h) | (mg/24 h) | (mg/24 h) | baseline (%) |
| 19-1 | 3936 | 2795 | 3648 | 2590 | −7.3 |
| 19-2 | 4800 | 3408 | 4800 | 3408 | 0.0 |
| 19-3 | 1536 | 1091 | 1824 | 1295 | 18.8 |
| 19-4^{a} | 3840 | 2726 | 4032 | 2863 | 5.0 |
| 19-5 | 2112 | 1500 | 2304 | 1636 | 9.1 |
| 19-6 | 1920 | 1363 | 1920 | 1363 | 0.0 |
| 19-7^{a} | 768 | 545 | 768 | 545 | 0.0 |
| 19-8 | 1152 | 818 | 1344 | 954 | 16.7 |
| 19-9^{a} | 1248 | 886 | 1344 | 954 | 7.7 |
| 19-10^{a} | 3264 | 2317 | 3264 | 2317 | 0.0 |
| 19-11^{a} | 1344 | 954 | 1536 | 1091 | 14.3 |
| 19-12 | 1344 | 954 | 1536 | 1091 | 14.3 |
| 19-13 | 1728 | 1227 | 1728 | 1227 | 0.0 |
| 19-14^{a} | 2880 | 2045 | 3072 | 2181 | 6.7 |
| 19-15 | 1152 | 818 | 1440 | 1022 | 25.0 |
| 19-16 | 2976 | 2113 | 3360 | 2386 | 12.9 |
| 19-17 | 576 | 409 | 768 | 545 | 33.3 |
| 19-18 | 2880 | 2045 | 2880 | 2045 | 0.0 |
| 19-19 | 3456 | 2454 | 3552 | 2522 | 2.8 |
| 19-20 | 3072 | 2181 | 3264 | 2317 | 6.3 |

^{a}Prematurely discontinued foslevodopa and foscarbidopa

As described, if a patient has a levodopa equivalent dose of 2000 mg prior to study entry, the recommended starting dose of the pharmaceutical composition of foslevodopa and foscarbidopa (levodopa 4′-monophosphate and carbidopa 4′-monophosphate) in a 20:1 w/w ratio is 4032 mg levodopa 4′-monophosphate delivered over 24 hours. As the levodopa dose for each patient is determined by individualized patient titration, patients in these studies start at doses of the pharmaceutical composition levodopa 4′-monophosphate prodrug and carbidopa 4′-monophosphate, which are expected to result in exposure close to their previous regimen with the option of further dose modification to achieve optimal clinical response. In this manner, personalized, titratable dosing can be achieved across a dose range to address therapeutic needs.

### Example 6: Full Prescribing Information

### 1. The Medicinal Product

Foslevodopa and foscarbidopa 240 mg/ml+12 mg/ml solution for infusion foscarbidopa/foslevodopa subcutaneous infusion (the product described herein).

### 2. Qualitative and Quantitative Composition

1 ml contains 240 mg foslevodopa and 12 mg foscarbidopa.

10 ml contain 2400 mg foslevodopa and 120 mg foscarbidopa. foslevodopa and foscarbidopa are prodrugs equivalent to approximately 170 mg levodopa and 9 mg carbidopa per 1 ml.

The pH is approximately 7.4.

Osmolality is approximately 2200 to 2500 mOsmol/kg but may range up to 2700 mOsmol/kg.

Excipient with Known Effect

foscarbidopa/foslevodopa subcutaneous infusion contains approximately 1.84 mmol (42.4 mg) sodium per ml.

### 3. Pharmaceutical Form

Solution for infusion (infusion).

foscarbidopa/foslevodopa subcutaneous infusion is a sterile, preservative-free, clear to slightly opalescent solution in a glass vial. The solution should be free from particulates. foscarbidopa/foslevodopa subcutaneous infusion may vary from colorless to yellow to brown and may have a purple or red tint. Variations in color are expected and have no impact on product quality. The solution may become darker in color after piercing of the vial stopper or while in the syringe.

### 4. Clinical Particulars

4.1 Therapeutic indications

Treatment of advanced levodopa-responsive Parkinson's disease with severe motor fluctuations and hyperkinesia or dyskinesia when available combinations of Parkinson medicinal products have not given satisfactory results.

### Indications and Usage

The product described herein (foscarbidopa/foslevodopa for subcutaneous infusion) is a combination of foscarbidopa (an aromatic amino acid decarboxylation inhibitor) and foslevodopa (an aromatic amino acid) indicated in adults for the treatment of motor fluctuations in patients with advanced Parkinson's disease.

### 4.2 Posology and Method of Administration

### Posoloqy

The subcutaneous infusion foscarbidopa/foslevodopa is administered as a continuous subcutaneous infusion, 24 hours per day.

The recommended starting daily dose of foscarbidopa/foslevodopa subcutaneous infusion is determined by converting the daily levodopa intake to levodopa equivalents (LE) and then increasing it to account for a 24-hour administration. The dose may be adjusted to reach a clinical response that maximizes the functional “On” time and minimizes the number and duration of “Off” episodes and “On” episodes with troublesome dyskinesia.

### Dosage and Administration

For subcutaneous administration only. Do not administer intravenously or intramuscularly.

Parenteral drug products should be inspected visually for particulate matter and discoloration prior to administration, whenever solution and container permit. Variations in color are expected. The solution will be colorless to yellow to brown and may have a purple or red tint.

The maximum recommended daily dose of the product described herein foscarbidopa/foslevodopa is about 4000 mg of foslevodopa ((2850 mg levodopa equivalents) administered over 24 hours. The recommended starting daily dose of the foscarbidopa/foslevodopa subcutaneous infusion is determined by converting the daily levodopa intake to levodopa equivalents (LE) and then adjusting it to account for a 24-hour administration. Titrate daily dose based on clinical response for the patient. Administer the foscarbidopa/foslevodopa subcutaneous infusion into the subcutaneous area, preferably in the abdominal area, using an infusion set and a subcutaneous infusion pump.

The foscarbidopa/foslevodopa subcutaneous infusion replaces levodopa-containing medications and catechol-O-methyl transferase (COMT)-inhibitors. If required, other medicinal products for Parkinson's disease can be taken concurrently.

### Contraindications

The foscarbidopa/foslevodopa subcutaneous infusion is contraindicated in patients taking nonselective monoamine oxidase (MAO) inhibitors.

### Initiation of Treatment

Patients selected for treatment with foscarbidopa/foslevodopa subcutaneous infusion should be capable of understanding and using the delivery system themselves or with assistance from a caregiver.

Patients should be trained on the proper use of foscarbidopa/foslevodopa subcutaneous infusion and the delivery system (see method of administration) prior to initiating treatment with foscarbidopa/foslevodopa subcutaneous infusion and, as necessary, thereafter.

Three steps are required to initiate treatment with foscarbidopa/foslevodopa subcutaneous infusion.
Step 1: Calculate the LE based on the levodopa-containing medications and COMT inhibitors used during the patient's awake time.
Step 2: Determine the hourly infusion rate of foscarbidopa/foslevodopa subcutaneous infusion.
Step 3: Determine the volume of the loading dose.

Step 1: Calculate the LE based on the levodopa-containing medications used during the patient's awake time.

The levodopa amount from all levodopa-containing formulations used during the waking time of the day should be converted to LE using the appropriate dose multiplying factor from Table 23 and then summed. For this calculation, only consider levodopa and COMT inhibitors. Do not include night-time dosing of either medication, and do not include rescue levodopa or any other anti-Parkinsonian medication or therapy in this calculation. If any COMT inhibitors are taken within a 24-hour period, regardless of the COMT inhibitor dose, a correction factor should be applied to the sum of LE as presented in Table 23.

TABLE 23
Calculating the Levodopa Equivalents (LE)

| | Dose |
|---|---|
| | Multiplication |
| Levodopa Formulation | Factor |
| Immediate-release, including enteral suspension | 1 |
| Sustained-release, controlled-release or prolonged- | 0.75 |
| release^{a} | |
| Extended-release capsules (Rytary ®) (mg) | |
| 0-855 | 0.42 |
| 856-1755 | 0.48 |
| 1756-2340 | 0.56 |
| 2341 or above | 0.67 |

If any COMT inhibitor is used, multiply sum
of calculated LE from above by 1.33^{a}
^{a}The levodopa contained in combined CD/LD/COMT-inhibitor formulations counts as immediate-release and needs to be added to the LE from all other sources of levodopa before the sum is multiplied for the COMT-inhibitors correction factor (i.e., do not apply COMT correction factor to single LE).
CD = carbidopa;
LD = levodopa;
COMT = catechol-O-methyl transferase;
LE = levodopa equivalents

Step 2: Determine the hourly infusion rate of foscarbidopa/foslevodopa subcutaneous infusion.

Refer to Table 24 for suggested foscarbidopa/foslevodopa subcutaneous infusion starting infusion rates based on the LE calculated in Step 1.

The hourly infusion rate for foscarbidopa/foslevodopa subcutaneous infusion in Table 24 is based on a patient's LE intake during a typical 16-hour awake time (LE16).

If the LE determined in Step 1 were based on an awake time either longer or shorter than 16 hours, the LE should be adjusted to a 16-hour period. To adjust to a 16-hour period, take the LE calculated in Step 1, divide by the number of hours the patient is typically awake, and then multiply by 16. Then refer to Table 24 for foscarbidopa/foslevodopa subcutaneous infusion suggested starting infusion rates.

The hourly infusion rate determined in this step should be entered as the Base infusion rate when programming the pump (refer to the pump instructions for use for details).

TABLE 24
Suggested foscarbidopa/foslevodopa subcutaneous
infusion starting hourly infusion rate

| LE₁₆ (LE from | Suggested foscarbidopa/foslevodopa |
|---|---|
| all oral LD-containing | subcutaneous infusion starting hourly |
| medications taken over 16-hour | infusion rate (ml/hr)^{a} |
| awake time (mg)) | administered over 24 hours |
| <400 | 0.15 |
| 400-499 | 0.15-0.17 |
| 500-599 | 0.17-0.20 |
| 600-699 | 0.20-0.24 |
| 700-799 | 0.24-0.27 |
| 800-899 | 0.27-0.30 |
| 900-999 | 0.30-0.34 |
| 1000-1099 | 0.34-0.37 |
| 1100-1199 | 0.37-0.40 |
| 1200-1299 | 0.40-0.44 |
| 1300-1399 | 0.44-0.47 |
| 1400-1499 | 0.47-0.51 |
| 1500-1599 | 0.51-0.54 |
| 1600-1699 | 0.54-0.57 |
| 1700-1799 | 0.57-0.61 |
| 1800-1899 | 0.61-0.64 |
| 1900-1999 | 0.64-0.68 |
| >2000 | 0.69 |

Assumptions used to generate the “Suggested foscarbidopa/foslevodopa subcutaneous infusion starting hourly infusion rate”:
Total daily LE over 16 hours are increased by 50% to account for 24-hour dosing
Subcutaneous foslevodopa is 8% more bioavailable than enterally absorbed levodopa
The molecular weight ratio between foslevodopa and levodopa is 1.41
One millilitre of foscarbidopa/foslevodopa subcutaneous infusion contains 240 mg of foslevodopa and 12 mg of foscarbidopa
Most patients with PD are treated with oral PD medications during their waking time (typically 16-hour/day treatment period); once the amount of foslevodopa needed over the 16 hour period has been calculated, it is divided by 240 mg to determine the number of millilitres needed over the 16-hour period, and then divided over 16 hours to establish the hourly infusion rate
^{a}The hourly infusion rate is calculated using the following formula,
Hourly infusion rate (ml/hr) = [(LEX · 0.92 · 1.41)/240]/X where X is the number of patient's awake hours used to determine the LE (e.g., X = 16, in the table above).
LE = levodopa equivalents;
LD = levodopa;
PD = Parkinson's disease

Step 3: Determine the volume of the loading dose.

A loading dose can be administered immediately prior to commencing the hourly infusion to quickly achieve symptomatic control when starting foscarbidopa/foslevodopa subcutaneous infusion therapy in an “Off” state (or if the pump has been off for more than 3 hours).

Table 25 provides the recommended loading dose volume (ml) of foscarbidopa/foslevodopa subcutaneous infusion to be programmed into the pump (refer to the pump instructions for use for details) and the corresponding amount, in milligrams, of immediate-release levodopa, regardless of the peripheral inhibitor of the DOPA decarboxylase (e.g., carbidopa, benserazide) co-administered.

TABLE 25
Determination of foscarbidopa/foslevodopa subcutaneous
infusion volume recommended for the loading dose

| Recommended loading | Approximate |
|---|---|
| dose volume (ml) to be | corresponding levodopa |
| programmed into the pump | amount (mg) |
| 0.6 | 100 |
| 0.9-1.2 | 150-200 |
| 1.5-1.8 | 250-300 |
| 2.0 | 350 |

Treatment with foscarbidopa/foslevodopa subcutaneous infusion may be initiated while patients are either in the “Off” state or in the “On” state. Patients initiating foscarbidopa/foslevodopa subcutaneous infusion therapy in the “On” state may start the infusion without the need for a foscarbidopa/foslevodopa subcutaneous infusion loading dose. Loading doses can be administered both via the pump or using oral levodopa tablets.

### Optimization and Maintenance

The healthcare professional may adjust the starting hourly infusion rate to achieve the optimal clinical response for the patient. The hourly infusion rate should be delivered continuously over the 24-hour daily infusion period. If desired, the healthcare professional can program and enable 2 alternative infusion rates (Low/High). All infusion rates may be adjusted in increments of 0.01 ml/hr (which is equivalent to approximately 1.7 mg of levodopa/hour) and should not exceed 1.04 ml/hr (or approximately 4260 mg levodopa/day [6000 mg of foslevodopa/day]). The infusion pump incorporates secure access to dose configurations to prevent patients from making changes to their pre-programmed flow rates or Extra Dose functionality.

The foscarbidopa/foslevodopa subcutaneous infusion can be taken alone or, if necessary, with other concurrent medicinal products for Parkinson's disease, based on the judgement of the healthcare professional. A reduction in other concomitant medications for Parkinson's disease, followed by an adjustment in foscarbidopa/foslevodopa subcutaneous infusion dosage, may be considered during foscarbidopa/foslevodopa subcutaneous infusion. The concomitant use of the foscarbidopa/foslevodopa subcutaneous infusion with other levodopa-containing medications or with medicinal products that significantly regulate synaptic dopamine levels (such as COMT inhibitors) has not been studied.

### Alternative Flow Rate

The infusion pump also allows for 2 alternative infusion rate options to be programmed for patient use. The alternative infusion rates must be enabled and pre-programmed by the healthcare professional and may be selected by patients to account for changes in functional demand, e.g., lowering the dosage at night-time or increasing the dose for prolonged intense.

### Extra Doses

If enabled by their healthcare professional, patients may self-administer an Extra Dose to manage acute “Off” symptoms experienced during continuous infusion.

### Method of Administration

The foscarbidopa/foslevodopa subcutaneous infusion is administered subcutaneously, preferably in the abdomen, avoiding a 5-cm radius area from the navel. The infusion set (cannula) can remain in place for up to 3 days when the medication is infused continuously. The infusion site was rotated and a new infusion set was used at least every 3 days. New infusion sites were recommended to be at least 2.5 cm from sites used within the previous 12 days.

In a Pharmacokinetic crossover study, administration of foscarbidopa/foslevodopa subcutaneous infusion via the arm and thigh resulted in nearly equivalent exposure to the abdomen (see section 5.2 Absorption). Long-term safety and efficacy of administration to the arm and thigh were not evaluated.

The medication should be stored and handled as described in the section referred to as “Handling and Storage”. The medication vials are for single dose only. Once the content of a vial is transferred into the syringe, the contents of the syringe should be administered within 24 hours. Used medication vials and syringes should be discarded according to local regulations. Syringes must be discarded, even if residual product remains, as instructed by the healthcare professional.

### Special Populations

### Gender or Race

Following foscarbidopa/foslevodopa subcutaneous infusion administration, carbidopa and levodopa exposures in both Japanese subjects and Han Chinese subjects were comparable to those in Caucasian subjects.

### 5. Pharmacological Properties

### 5.1 Pharmacodynamic Properties

Pharmacotherapeutic group: Anti-Parkinson drugs, foslevodopa and decarboxylase inhibitor

### Mechanism of Action

The product described herein, (foslevodopa/foscarbidopa) 240 mg/12 mg per ml solution for infusion, is a prodrug combination of levodopa monophosphate and carbidopa monophosphate (ratio 20:1) in a solution for 24 hour/day continuous subcutaneous infusion in advanced Parkinson's disease patients who are not adequately controlled with current medical therapy. Foslevodopa and foscarbidopa are converted in-vivo to levodopa and carbidopa. Levodopa relieves symptoms of Parkinson's disease following decarboxylation to dopamine in the brain. Carbidopa, which does not cross the blood-brain barrier, inhibits the extracerebral decarboxylation of levodopa to dopamine, which means that a larger amount of levodopa becomes available for transportation to the brain and transformation into dopamine.

### Pharmacodynamic Effects

foscarbidopa/foslevodopa subcutaneous infusion subcutaneous administration and Duodopa intestinal administration were shown to have comparable levodopa Cₘₐₓ, AUC, and degree of fluctuation, which supports a comparable efficacy profile. By achieving same concentrations of levodopa as Duodopa, foscarbidopa/foslevodopa subcutaneous infusion reduces the motor fluctuations and increases the “On”-time in levodopa-responsive patients with advanced Parkinson's disease. The motor fluctuations and hyperkinesia or dyskinesia are reduced because the plasma concentrations of levodopa are being kept at a steady level within the individual therapeutic window. Therapeutic effect on motor symptoms (“On” state) is achieved on the first treatment day.

### Clinical Efficacy and Safety

Studies with Duodopa Intestinal Gel Formulation

The efficacy of Duodopa intestinal gel was confirmed in two identically-designed Phase 3, 12-week, randomised, double-blind, double-dummy, active-controlled, parallel group, multicentre studies to evaluate the efficacy, safety, and tolerability of the Duodopa intestinal gel system against levodopa/carbidopa 100/25 mg tablets. The studies were conducted with patients with advanced Parkinson's disease who were levodopa-responsive and had persistent motor fluctuations despite optimised treatment with oral levodopa/carbidopa and other available anti-Parkinson's disease medications and enrolled a total of 71 patients. The results of the two studies were combined and a single analysis was conducted.

The primary efficacy endpoint, change in normalised “Off” time (baseline to endpoint) based on Parkinson's Disease Diary (PD Diary) data using last observation carried forward demonstrated a statistically significant least square (LS) mean difference in favour of the Duodopa treatment group (Table 26).

The primary end point results were supported by a Mixed Model Repeated Measures (MMRM) analysis which examined the change from baseline to each post-baseline study visit. This analysis of “Off” time demonstrated a statistically significant greater improvement of the Duodopa group over the Active control group at Week 4, and that improvement was shown to be statistically significant at Weeks 8, 10, and 12.

This change in “Off” time was associated with a statistically significant LS mean difference from baseline in the average daily normalised “On” time without troublesome dyskinesia between the Duodopa intestinal gel treatment group and the active control group based on PD Diary data. The baseline values were collected three days prior to randomisation and after 28 days of oral therapy standardisation.

TABLE 26
Change from baseline to endpoint in “off” time and in
“on” time without troublesome dyskinesia

| | | | | | | |
|---|---|---|---|---|---|---|
| | | | | | LS | |
| | | Base- | End- | LS | mean | |
| | | line | point | mean | (SE) | |
| | | mean | Mean | (SE) of | of dif- | |
| Treatment | | (SD) | (SD) | change | ference | P |
| Group | N | (hours) | (hours) | (hours) | (hours) | value |

Primary measure

| | | | | | | |
|---|---|---|---|---|---|---|
| “Off” time | 31 | 6.90 | 4.95 | −2.14 | | |
| Active control^{a} | | (2.06) | (2.04) | (0.66) | | |
| Duodopa | 35 | 6.32 | 3.05 | 4.04 | −1.91 | 0.0015 |
| intestinal gel | | (1.72) | (2.52) | (0.65) | (0.57) | |

Secondary measure

| | | | | | | |
|---|---|---|---|---|---|---|
| “On” time without | 31 | 8.04 | 9.92 | 2.24 | | |
| troublesome | | (2.09) | (2.62) | (0.76) | | |
| dyskinesia | | | | | | |
| Active control | | | | | | |
| Duodopa | 35 | 8.70 | 11.95 | 4.11 | 1.86 | 0.0059 |
| intestinal gel | | (2.01) | (2.67) | (0.75) | (0.65) | |

SD = standard deviation;
SE = standard error
^{a}Active control, oral levodopa/carbidopa 100/25 mg tablets (Sinemet ® tablets over-encapsulated)

Analyses of other secondary efficacy endpoints, in order of the hierarchical testing procedure, demonstrated statistically significant results for Duodopa intestinal gel compared to oral levodopa/carbidopa for the Parkinson's Disease Questionnaire (PDQ-39) Summary Index (an index Parkinson's disease-related quality of life), Clinical Global Impression-Improvement (CGI-1) score, and Unified Parkinson's Disease Rating Scale (UPDRS) Part II score (Activities of Daily Living). The PDQ-39 Summary Index showed a decrease from baseline of 10.9 points at week 12 for Duodopa intestinal gel group. Other secondary endpoints UPDRS Part Ill score, EuroQol 5-dimensions Questionnaire (EQ-5D) Summary Index, and Zarit Burden Interview (ZBI) total score, did not meet statistical significance based on the hierarchical testing procedure.

A Phase 3, open-label, single-arm, multicentre study was conducted to assess the long-term safety and tolerability of Duodopa over 12 months in 354 patients. The target population was levodopa-responsive patients with advanced Parkinson's disease and motor fluctuations despite optimised treatment with available Parkinson's disease medications. The average daily normalised “Off” time changed by −4.44 hours from Baseline to Endpoint (6.77 hours at Baseline and 2.32 hours at Endpoint) with a corresponding 4.8 hour increase in “On” time without troublesome dyskinesia.

A Phase 3, open-label, randomised, multicentre study was conducted to assess the effect of Duodopa intestinal gel on dyskinesia compared with optimised medical treatment (OMT) over 12 weeks in 61 patients. The target population was levodopa-responsive patients with advanced PD and motor fluctuations inadequately controlled with OMT and with a baseline Unified Dyskinesia Rating Scale (UDysRS) Total Score≥30. The change from baseline to Week 12 in UDysRS total score (primary efficacy endpoint) demonstrated a statistically significant LS Mean difference (−15.05; P<0.0001) in favour of the Duodopa treatment group compared with OMT group. Analysis of secondary efficacy endpoints using a fixed sequence testing procedure, demonstrated statistically significant results in favour of Duodopa compared with OMT for “On” time without troublesome dyskinesia as measured by PD Diary, for Parkinson's Disease Questionnaire-8 (PDQ-8) summary index, Clinical Global Impression Change (CGI-C) score, UPDRS Part II score, and for “Off” time as measured by PD Diary. The UPDRS Part Ill score did not meet statistical significance.

Studies with foscarbidopa/foslevodopa subcutaneous infusion

Foslevodopa and foscarbidopa is a prodrug combination of levodopa monophosphate and carbidopa monophosphate (ratio 20:1) in solution intended for 24-hour/day continuous subcutaneous infusion. Subcutaneous foscarbidopa/foslevodopa infusion administration and Duodopa intestinal administration were shown to have comparable levodopa Cₘₐₓ and AUC parameters, which supports a comparable efficacy profile. The study showed stable levodopa exposure with fluctuation values of 0.262 and 0.404 for foscarbidopa/foslevodopa subcutaneous infusion and Duodopa, respectively (see section 5.2 Pharmacokinetic properties).

A Phase 3, open-label, single-arm study was conducted to evaluate the safety and tolerability of 24-hour daily exposure of continuous subcutaneous infusion of foscarbidopa/foslevodopa subcutaneous infusion over 52 weeks in 223 patients. The target population was levodopa-responsive patients with Parkinson's disease whose motor symptoms that were inadequately controlled with current treatment who experienced a minimum of 2.5 hours of “Off” time per day as assessed by Parkinson's disease (PD) diaries. The dose conversion from oral medications to foscarbidopa/foslevodopa subcutaneous infusion was achieved with one outpatient office visit.

The mean daily normalised “Off” time (PD Diary) decreased from 5.79 hours at baseline to 2.85 hours at Week 26, for a mean improvement of 2.94 hours. This change in “Off” time was associated with a mean increase of 3.24 hours from baseline in “On” time without troublesome dyskinesia. The mean increase from baseline in “On” time without dyskinesia was 4.00 hours. The percentage of patients with morning akinesia, measured as the first symptom reported upon awakening as derived from PD Diary, decreased from 77.8% at baseline to 20.8% at Week 26. There was meaningful improvement in other secondary endpoints: motor aspects of experiences of daily living (MDS-UPDRS Part II score), sleep symptoms (Parkinson's Disease Sleep Scale-2 [PDSS-2] total score), quality of life (PDQ-39 and EQ-5D-5L summary indices). (Table 27).

TABLE 27
Change from baseline to week 26 in efficacy endpoints

| | | | | |
|---|---|---|---|---|
| | | Baseline | Week 26 | Mean |
| | | mean | Mean | change |
| Measure | N | (SD) | (SD) | (SD) |

| | | | | | | | |
|---|---|---|---|---|---|---|---|
| “Off” time (hours)^{a} | 97 | 5.79 | (2.35) | 2.85 | (3.02) | −2.94 | (3.16) |
| “On” time without troublesome | 97 | 9.62 | (2.42) | 12.87 | (3.08) | 3.24 | (3.16) |
| dyskinesia (hours)^{a} | | | | | | | |
| “On” time without dyskinesia | 97 | 7.01 | (3.39) | 11.02 | (4.23) | 4.00 | (4.42) |
| (hours)^{a} | | | | | | | |
| Motor aspects of experiences of | 104 | 15.9 | (7.17) | 12.7 | (7.65) | −3.2 | (6.82) |
| daily living^{b} | | | | | | | |
| Sleep symptoms^{c} | 104 | 20.6 | (9.93) | 15.0 | (9.64) | −5.7 | (11.20) |
| Quality of life^{d} | 104 | 34.5 | (14.93) | 27.3 | (15.05) | −7.2 | (11.36) |
| Health-related quality of life^{e} | 86 | 0.662 | (0.184) | 0.744 | (0.1375) | 0.082 | (0.1712) |

| | | Baseline | Endpoint | Difference |
|---|---|---|---|---|
| Measure | | (%) | (%) | (%) |
| Morning akinesia (%)^{f} | 77 | 77.8% | 20.8% | −57.0% |

SD = standard deviation.
^{a}Parkinson's disease (PD) Diary.
^{b}Movement Disorder Society-Unified Parkinson's Disease Rating Scale (MDS-UPDRS) Part II score.
^{c}Parkinson's Disease Sleep Scale-2 (PDSS-2) total score.
^{d}Parkinson's Disease Questionnaire-39 items (PDQ-39) summary index.
^{e}EuroQol 5-dimensions Questionnaire (EQ-5D-5L) summary index.
^{f}% of subjects with early morning “Off” status based on the first morning symptom upon awakening derived from PD Diary.

### 5.2 Pharmacokinetic Properties

### Absorption

The foscarbidopa/foslevodopa subcutaneous infusion is administered directly into the subcutaneous space and is converted to levodopa and carbidopa. In a phase 1 study in healthy volunteers, levodopa and carbidopa were detectable in plasma within 30 minutes at the first pharmacokinetic collection point. In most subjects the steady state was achieved within 2 hours when foscarbidopa/foslevodopa subcutaneous infusion dosing was delivered as loading dose followed by continuous infusion.

To determine absorption of foscarbidopa/foslevodopa subcutaneous infusion at different subcutaneous sites, healthy volunteers were administered foscarbidopa/foslevodopa subcutaneous infusion to the abdomen, arm and thigh using a 3-way crossover design. Pharmacokinetic analysis from this study showed that the 3 sites have nearly identical levodopa and carbidopa exposure suggesting foscarbidopa/foslevodopa subcutaneous infusion absorption is similar at the different subcutaneous sites.

The foscarbidopa/foslevodopa subcutaneous infusion bypasses the gut, so food does not change absorption or systemic exposure of levodopa/carbidopa, therefore food is not expected to have any impact on the bioavailability or systemic exposure of the foscarbidopa/foslevodopa subcutaneous infusion.

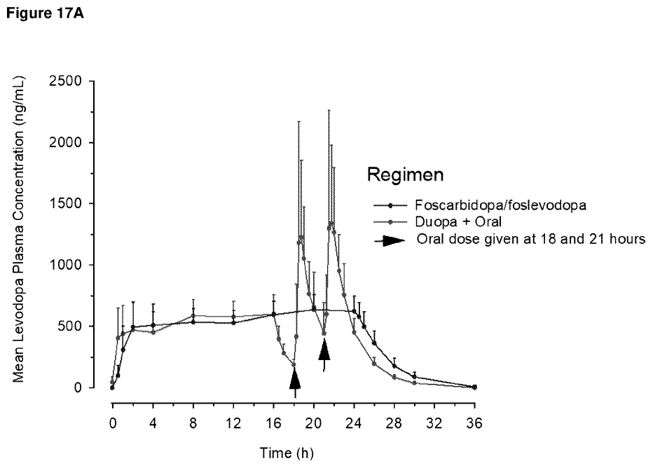

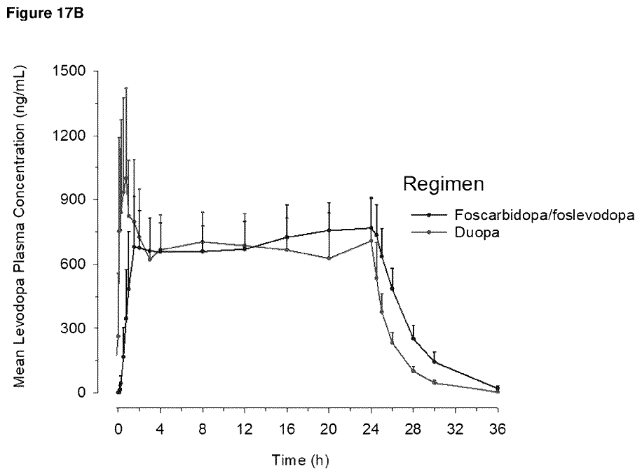

Following foslevodopa and foscarbidopa administration in healthy volunteers, levodopa steady state is achieved in approximately 2 hours and maintained during the infusion period. For the PK comparability study between the product disclosed herein (foscarbidopa/foslevodopa for subcutaneous infusion) and Duopa (carbidopa/levodopa for enteral suspension) with night-time oral medication, levodopa steady state was achieved rapidly and maintained during the infusion period following administration of the product of the present disclosure in healthy volunteers. Results from the study show that the levodopa PK is comparable for both regimens. FIG. 17A shows levodopa exposure following both 24-hour administration of the present product and 16-hour Duopa administration followed by night-time oral levodopa/carbidopa dosing. A follow up PK comparability study in patients with advanced PD was completed to assess the levodopa PK comparability between Duopa and the present product, both infused for 24 hours. Results from the study show that the levodopa PK is comparable for both regimens based on AUC (FIG. 17B).

### Distribution

The partitioning ratio for levodopa between erythrocytes and plasma is approximately 1. Levodopa has negligible binding to plasma proteins (<10%). Levodopa is transported into the brain by the carrier mechanism for large neutral amino acids.

Carbidopa is approximately 36% bound to plasma protein. Carbidopa does not cross the blood-brain barrier.

Both foslevodopa and foscarbidopa have low binding to plasma proteins (24%-26%).

### Metabolism and Excretion/Biotransformation and Elimination

The foscarbidopa/foslevodopa subcutaneous infusion prodrugs are rapidly converted by phosphatases into carbidopa and levodopa and thus the prodrugs are removed quickly from circulation. Levodopa is mainly eliminated via metabolism by the aromatic amino acid decarboxylase (AAAD) and the catechol-O-methyl-transferase (COMT) enzymes. Other routes of metabolism are transamination and oxidation. The decarboxylation of levodopa to dopamine by AAAD is the major enzymatic pathway when no enzyme inhibitor is co-administered. O-methylation of levodopa by COMT forms 3-O-methyldopa. When administered with carbidopa, the elimination half-life of levodopa is approximately 1.5 hours. Carbidopa is metabolized to two main metabolites (α-methyl-3-methoxy-4-hydroxyphenylpropionic acid and α-methyl-3,4-dihydroxyphenylpropionic acid). These 2 metabolites are primarily eliminated in the urine unchanged or as glucuronide conjugates. Unchanged carbidopa accounts for 30% of the total urinary excretion. The elimination half-life of carbidopa is approximately 2 hours.

### Effect of Food

Levodopa is transported by a saturable amino acid transporter system active in the small intestine and at the blood-brain barrier. The absorption of Foslevodopa and Foscarbidopa occurs subcutaneously rather than via the gastrointestinal tract. Food is not expected to have any impact on the bioavailability or systemic exposure of Foslevodopa and Foscarbidopa.

### Example 7: Foslevodopa and Foscarbidopa Maintains Equivalent Levodopa Exposure to Levodopa-Carbidopa Intestinal Gel Delivered to the Jejunum

### Introduction

The Phase 1 study characterized the LD PK profile following 24-hour foslevodopa/foscarbidopa CSCI and 16-hour LCIG jejunal infusion with night-time oral LD/CD dosing in healthy volunteers. The LCIG and oral LD/CD regimen was selected as an example of how a PD patient who experiences night-time symptoms and is in treatment with LCIG may use the product.

### How Supplied

The product of the present disclosure is supplied in a single-dose glass vial filled with approximately 10 mL of solution. Each vial contains a colorless to yellow to brown (may have a purple or red tint), and clear to slightly opalescent solution. Each glass vial is fitted with a grey rubber stopper, aluminum crimp cap, and turquoise plastic flip-off cap. The rubber stopper on the vial does not contain natural rubber latex.

For administration of the product, only the pump disclosed herein should be used (refer to the pump instructions for use for details) using sterile, single-patient-use infusion components (syringe, infusion set, and vial adapter) that are qualified for use and provided separately.

### Storage and Handling

Do not use the product beyond the expiration date on the vial/carton label. Keep the medication vials in the outer carton to protect the vials from breaking. Store the product refrigerated at 36° F. to 46° F. (2° C. to 8° C.). The product may be stored at room temperature up to a maximum of 86° F. (30° C.) for a single period of up to 28 days. Once the product has been stored at room temperature, do not return the product to the refrigerator. Discard the product if not used within the 28-day room temperature period. Record the date when the product is first removed from the refrigerator in the space provided on the carton. Do not freeze. Do not use any solution that has been frozen. Do not shake. Do not dilute. Do not mix the product with other products. Use aseptic technique when preparing and administering this product. The medication vials are for single dose only. The entire contents of the product vial should be transferred into a syringe for administration. Do not withdraw only a partial portion of the vial contents. Discard the vial after transfer of the product to the syringe. Discard the syringe and any unused product in the syringe after the product has been in the syringe for 24 hours. Discard all used administration supplies and any unused product immediately after each infusion in accordance with local requirements.

### Methods:

### Study Design and Eligibility

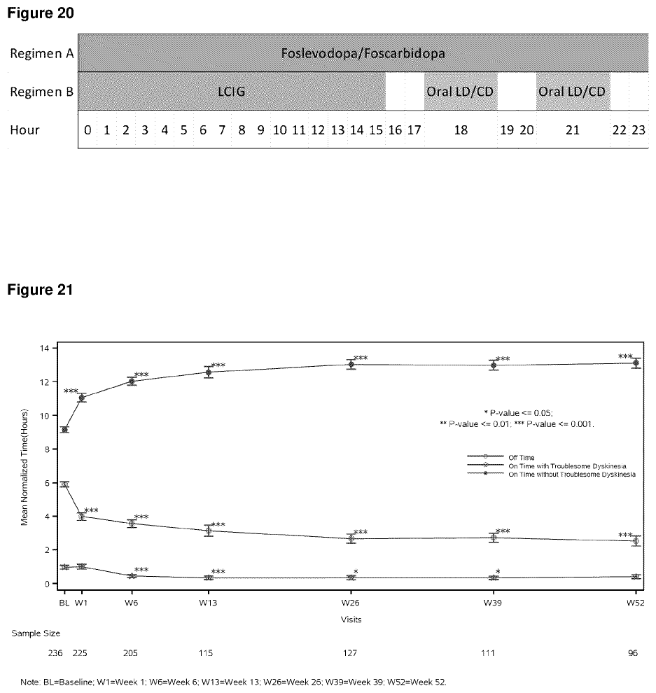

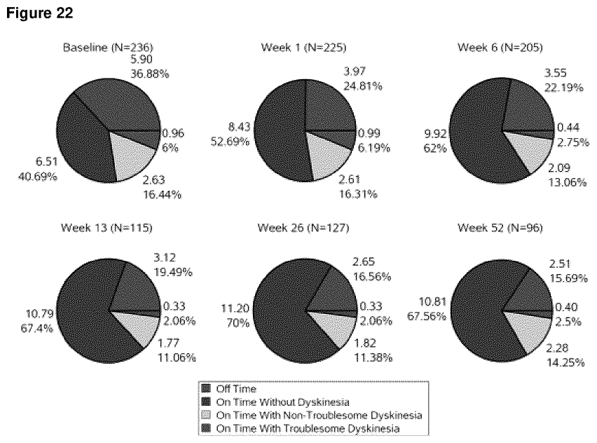

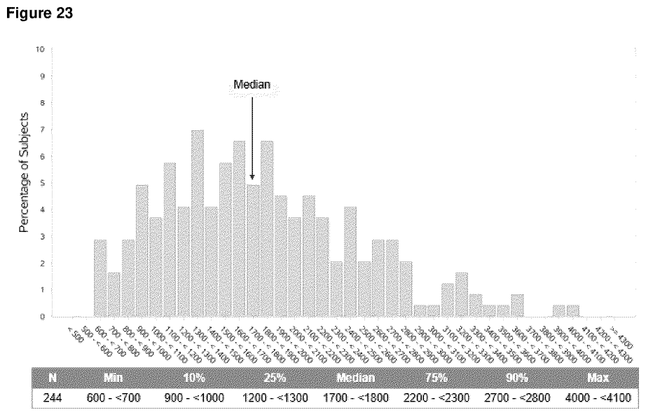

This was a Phase 1, open-label, randomized, 2-period crossover study conducted in healthy volunteers (45-75 years old). A total of 25 subjects (N=25) who were in general good health based upon the results of a medical history, physical examination, vital signs, laboratory profile, and an electrocardiogram (ECG) participated in the study and were randomly assigned in equal numbers to one of the two sequences, Regimens A or B, as shown in FIG. 20. For Regimen A, foslevodopa/foscarbidopa was delivered subcutaneously into the abdomen via a portable infusion pump. Dosing consisted of a loading dose (80/4 mg foslevodopa/foscarbidopa) followed by a continuous infusion (700/35 mg foslevodopa/foscarbidopa) at a constant rate for a total infusion duration of 24 hours. For Regimen B, LCIG was delivered via a portable infusion pump through a nasojejunal (NJ) tube and consisted of a loading dose (50/12.5 mg LD/CD) followed by a continuous dose (350/87.5 mg LD/CD) at a constant rate for a total infusion duration of 16 hours followed by two oral immediate release LD/CD doses (100/25 mg LD/CD), one administered at 18 hours, and one administered at 21 hours after the start of infusion.

The study protocol and informed consent form were approved by the Institutional Review Board at each participating site prior to the initiation of any screening or study-specific procedures. Written informed consent was obtained from each individual participating in the study. The study was conducted in accordance with the Declaration of Helsinki and Good Clinical Practice guidelines of 1975, as defined by the International Conference on Harmonization.

### Study Procedures

In each period, for each regimen, drug administration began on the morning of Day 1. The initiations of infusions of foslevodopa and foscarbidopa in the two periods were at least 72 hours apart. Subjects were confined for 8 days and fasted for at least 4 hours prior to receiving foslevodopa and foscarbidopa and until at least 1 hour after the start of infusion. For Regimen B (LCIG plus oral LD/CD), on Day −1 of each period, an NJ tube was inserted endoscopically or with interventional radiology into position in the jejunum just beyond the ligament of Treitz. Proper placement was confirmed radiologically. The NJ tube was removed approximately 30 minutes after LCIG infusion was completed. Subjects were discontinued from the study if the placement of the NJ tube was unsuccessful or if dosing accuracy was thought to be compromised (e.g., kink in the NJ tube, pump high pressure alarms, leaking infusion set, emesis following oral dosing, etc.). Similarly, For Regimen A (foslevodopa/foscarbidopa regimen), subjects were discontinued from the study if dosing accuracy was thought to be compromised (e.g., kink in the tube, pump high pressure alarms, drug leaking at infusion site, bent cannula, etc.). Discontinued subjects were replaced with new subjects and assigned to the respective regimens.

Safety and tolerability evaluations were conducted throughout the entire duration of the study and included adverse event (AE) monitoring, physical examinations, vital sign measurements, electrocardiogram (ECG) variables, Columbia-Suicide Severity Rating Scale (C-SSRS), assessment of local skin reactions, and clinical laboratory testing (hematology, chemistry, and urinalysis). The Infusion Site Evaluation Scale, a two-part numeric (0-7) and letter (A-G) grade scale, where 7 and G indicated the worst outcomes, was used to assess skin tolerability. For both parts (numeric grade and letter grade), a frequency table was provided for the two scheduled times of evaluation (prior to beginning of foslevodopa and foscarbidopa infusion and at the completion of foslevodopa and foscarbidopa infusion).

### Pharmacokinetic Sampling and Assessments

Serial blood samples for LD, CD, LDP, CDP, and 3-O-methyldopa (3-OMD) were collected prior to priming through 36 hours after initiation of foslevodopa and foscarbidopa infusion on Day 1 of each period. Sampling for Regimen A were collected prior to priming, prior to infusion and at 0.5, 1, 2, 4, 8, 12, 16, 20, 24, 24.5, 25, 26, 28, 30 and 36 hours after start of infusion. Regimen B samples were collected prior to priming, prior to infusion and at 0.5, 1, 2, 4, 8, 12, 16, 16.5, 17, 18, 18.25, 18.5, 18.75, 19, 19.5, 20, 21, 21.25, 21.5, 21.75, 22, 22.5, 23, 24, 26, 28, 30 and 36 hours after start of infusion. Oral doses were taken at 18 and 21 hours after the start of infusion.

Plasma concentrations of LD, CD, LDP, CDP, and 3-OMD were determined using a validated liquid chromatography method with tandem mass spectrometric detection. The lower limits of quantitation (LLOQ) for LD, CD, LDP, CDP, and 3-OMD were established at 9.27 ng/mL, 9.99 ng/mL, 3.00 ng/mL, 8.97 ng/mL, and 400 ng/mL, respectively.

A linear mixed effects model was used to perform an analysis for the primary variables LD AUC0-16, AUC∞ and Cmax0-16. The model included fixed effects for period, regimen, and sequence. The subjects were viewed as a random sample. A point estimate and 90% confidence interval for the ratio of the central value for the foslevodopa/foscarbidopa regimen to the central value for the reference LCIG regimen was obtained by exponentiation of the point estimate and 90% confidence limits for the difference of the corresponding logarithm means. The 90% confidence interval was used to perform the two one-sided tests procedure at significance level 0.050. Equivalent LD exposure between the 2 regimens was predefined as the point estimate and 90% confidence interval of the ratio ([foslevodopa/foscarbidopa]:LCIG) of Cmax and AUC exposure parameters between 0.8 and 1.25.

For LD Cmax and two degree of fluctuation (DFL) variables (for hours 2 to 16 and hours 2 to 24), defined as (Cmax−Cmin)/Cave, the linear mixed effects model described for the primary analysis was again used to perform an analysis. The logarithmic transformation was employed for Cmax. A transformation was considered separately for each DFL variable, with attention primarily to whether the probability distributions appeared to have a considerable degree of non-symmetry, e.g., greater than 1.0 in magnitude.

### Results:

### Demographics

Sixteen males and nine females were enrolled in the study. The mean age of the 25 subjects was 57.2 years (range 46-70) and the mean weight was 78.2 kg (range 61-94). Subjects had a mean height of 171 cm (range 151-187). Twenty subjects were white (80%), three subjects were black (12%), one subject was Asian (4%), and one subject was multiple raced (4%).

### Pharmacokinetics

The LD exposure difference between the two regimens was less than 8% and well contained within the defined equivalence range (Table 28). The regimens differed little with respect to LD AUC₀₋₁₆ and AUC∞. The difference between the central values of the two regimens were not significant and the larger percentage difference between the regimens was that for LD AUC₀₋₁₆ (estimate of ratio of foslevodopa/foscarbidopa regimen central value to reference regimen central value was 0.951, 4.9% difference). The central value of LD Cmax0-16 of the foslevodopa/foscarbidopa regimen was lower than that of the reference regimen. The point estimates of the central values were 605.6 ng/mL and 656.4 ng/mL for the foslevodopa/foscarbidopa regimen and reference regimen, respectively, for a ratio of 0.923. For the overall LD Cmax, the point estimates were 658 ng/mL and 1874 ng/mL for the foslevodopa/foscarbidopa regimen and reference regimen, respectively.

Analyses were also performed for DFL2-16 (DFL for the time when both LCIG and foslevodopa/foscarbidopa infusions are ongoing) and DFL2-24 (DFL from 2 to 24 hours after the beginning of foslevodopa and foscarbidopa administration) for LD. The foslevodopa/foscarbidopa regimen had a smaller DFL2-16(0.344±0.319) compared to LCIG DFL2-16(0.453±0.251) for the time when both infusions were ongoing. The foslevodopa/foscarbidopa DFL₂₋₂₄(0.405±0.283) was considerably lower compared to the LCIG+oral LD/CD DFL₂₋₂₄(3.01±0.811) since Oral LD/CD dosing period was included in the DFL calculation. The CSCI of foslevodopa/foscarbidopa provided equivalent LD levels to LCIG infusion over the 16-hour interval and maintained those levels throughout the night-time (FIG. 17). The overall LD exposures over 24 hours also showed equivalence between CSCI of foslevodopa/foscarbidopa and LCIG+oral LD/CD.

### Safety

Twenty of the subjects completed both periods of the study and five subjects discontinued the study. The proportion of subjects who reported at least one treatment-emergent adverse event was higher following foslevodopa/foscarbidopa infusion (19/24, 79%) compared to LCIG infusion (20/4, 20%), mainly driven by infusion site reactions, which were reported in 18 of the 24 subjects who received foslevodopa/foscarbidopa. The events were mild in severity and scored 0 or 1 and A at the two-part Infusion Site Evaluation scale. One subject discontinued from the study due to a serious adverse event of seizure which was considered by the investigator to have no reasonable possibility of being related to foslevodopa and foscarbidopa. All adverse events considered to be “possibly related” to foslevodopa and foscarbidopa were mild and did not lead to discontinuation from the study.

### Discussion

The current study characterized the LD PK profiles of 24-hour CSCI of foslevodopa/foscarbidopa and 16-hour LCIG jejunal infusion with night-time oral LD/CD dosing. foslevodopa/foscarbidopa is intended for 24-hour continuous infusion to help manage night-time motor symptoms of PD as well as minimize morning “Off” symptoms. LCIG is typically infused during each patient's waking hours while night-time motor symptoms are often managed by supplemental oral LD/CD dosing. In this study, two tablets of LD/CD 100/25 mg were administered orally at night 3 hours apart to provide LD exposure throughout the night. In clinical practice, PD patients who interrupt LCIG administration before going to sleep may choose to supplement their therapy with oral LD/CD differently based on their individual needs.

### Pharmacokinetics

PD patients receiving therapeutic doses of LCIG have been shown to reach an efficacious steady state LD concentration ranging from approximately 1500 to 4000 ng/mL [6] with an average of approximately 2910 ng/mL [7]. More recently, the LD and CD PK profiles following four different therapeutic doses of foslevodopa/foscarbidopa delivered as four predefined CSCI rates were characterized in PD patients and showed that an efficacious LD steady state exposure ranged from approximately 750 to 4700 ng/mL; these data support the ability of foslevodopa/foscarbidopa to deliver the wide range of LD exposures needed to treat the vast majority of PD patients. In the current study, foslevodopa/foscarbidopa SC infusion was shown to provide equivalent LD exposure to LCIG infusion with and without night-time oral LD/CD dosing. The LD exposure following administration of foslevodopa/foscarbidopa 35/700 mg over 24 hours was comparable to that of LCIG 350/87.5 mg LD/CD over 16 hours followed by two 100/25 mg LD/CD oral doses at 18 and 21 hours after the start of infusion. The doses selected for this study were chosen to be in a tolerable range for healthy volunteers and still therapeutically relevant for patients with early stages of PD. A previous study, where progressively more severe PD patients were grouped in 4 categories based on their total amount of LD intake, demonstrated that LD exposure increases in direct proportion to the dose of foslevodopa/foscarbidopa administered (dose proportionality) indicating the exposure comparability is relevant at higher foslevodopa/foscarbidopa doses as well [8].

In this study, the LD degree of fluctuation was consistently low for both foslevodopa/foscarbidopa infusion and LCIG infusion for the first 16-hours. This shows that foslevodopa/foscarbidopa SC infusion is able to maintain LD exposure within a narrow therapeutic window needed to treat aPD patients. As a reference, Yeh et al.[9] previously reported a LD degree of fluctuation of 4.3±0.9 in healthy elderly volunteers following immediate release oral LD/CD, and Hauser et al. [10] reported 3.2±1.3 in PD patients. Oral LD administration for aPD is often hampered by variable LD plasma concentrations which result from an unpredictable absorption of the oral LD tablets. This variability can be attributed primarily to erratic gastric emptying [11] and to the short half-life of LD, even in the presence of CD [12, 13]. The advantage of continuous LD infusion compared to oral immediate release LD/CD has previously been demonstrated in aPD patients using LCIG [3] and this study has shown that the CSCI of foslevodopa/foscarbidopa achieves stable therapeutically relevant LD exposures across multiple dose regimens without the need for surgery or concerns of a food effect on LD absorption, since the subcutaneous delivery does not involve the gastrointestinal system. Moreover, the foslevodopa/foscarbidopa delivery system accepts adjustments of the infusion rate by small increments allowing the delivery of doses that can be individually tailored to the patient needs and that can be predicted in a dose proportional manner [8], finally providing optimized control of PD symptoms and motor fluctuations.

### Safety

Upon subcutaneous delivery, foslevodopa and foscarbidopa undergo enzymatic conversion to the active forms of LD and CD. While the doses of foslevodopa/foscarbidopa in this study were chosen to be in the lower end of the therapeutic range in order to be tolerated by healthy volunteers, another study showed dose proportionality between foslevodopa doses and LD plasma exposures and the systemic safety profile of foslevodopa is expected to be consistent with that of all other LD-containing medications. To this extent, no clinically significant laboratory measurements, vital signs, suicidal behaviors/ideation or other systemic events were observed during the course of this study, in line with what expected by the low dose of LD administered. At the same time, consistent with what previously reported by other medications that are delivered as a continuous subcutaneous infusion mild infusion site reactions were reported in subjects who received foslevodopa/foscarbidopa. Data from diabetic adults and children receiving insulin as CSCI indicates that while adverse events associated with insulin pumps are common, overall rates of permanent discontinuation due to those events are low Similarly, clinical experience in countries where the CSCI of the dopamine agonist apomorphine is approved for the management of motor fluctuations in aPD, found that infusion site reactions (including nodules) rarely result in discontinuation, and may be prevented by rotating infusion sites, correctly using aseptic techniques, and avoiding equipment reuse While no new safety issues were identified from this study, the importance of education in aseptic techniques and in the right application of the infusion set is crucial to guarantee an overall positive patient experience and minimize skin reactions.

TABLE 28
Point Estimates for Ratio of Central Values and
90% Confidence Intervals for Pharmacokinetic
Comparability Assessment of foslevodopa/foscarbidopa
Relative to Reference LCIG Regimen

Ratio of Central Values

| PK Parameter | Point Estimate | 90% Confidence Interval |
|---|---|---|
| C_{max 0-16} | 0.923 | 0.873-0.975 |
| AUC₀₋₁₆ | 0.951 | 0.893-1.012 |
| AUC_{inf} | 1.009 | 0.969-1.049 |
| C_{max 0-16} | 0.923 | 0.873-0.975 |
| AUC₀₋₁₆ | 0.951 | 0.893-1.012 |
| AUC_{inf} | 1.009 | 0.969-1.049 |

For the first 16-hours of the foslevodopa/foscarbidopa infusion and LCIG infusion, the LD degree of fluctuation was consistently low for both regimens (Table 29). This shows that foslevodopa/foscarbidopa SC infusion is able to maintain LD exposure.

TABLE 29
Summary of Levodopa Degree of Fluctuation Parameters

| | Degree of Fluctuation |
|---|---|
| Formulation | (±SD) |
| LCIG + Oral | 0.45 ± 0.25 |
| foslevodopa/foscarbidopa | 0.35 ± 0.34 |

Degree of fluctuation was determined as: (maximum LD plasma concentration-minimum LD plasma concentration)/average LD plasma concentration.
For comparison, following immediate release oral LD/CD, Yeh et al² previously reported a much higher LD degree of fluctuation of 4.3±0.9 in healthy elderly volunteers and Hauser et al³ reported 3.2±1.3 in PD patients.
All adverse events considered to be “possibly related” to foslevodopa and foscarbidopa were mild and did not lead to discontinuation from the study.

TABLE 30
Pharmacokinetic Parameters of LDP, CDP, and
3-OMD for Subjects Who Completed Both LCIG
and foslevodopa/foscarbidopa Regimens

| Pharmacokinetic | Regimen A | Regimen B |
|---|---|---|
| Parameters (units) | (N = 20) | (N = 20) |

| | | LDP | | | |
|---|---|---|---|---|---|
| Cₘₐₓ | ng/mL | — | | 97.9 | (107, 47) |
| Tₘₐₓ^{a} | h | — | | 8.0 | (0.5-24.0) |
| | | 3-OMD | | | |
| Cₘₐₓ | ng/mL | 3000 | (3080, 24) | 2460 | (2510, 21) |
| Tₘₐₓ^{a} | h | 24.0 | (19.0-26.0) | 25.0 | (24.0-28.0) |

^{a}Median (minimum-maximum).

### Example 8: Protocol for Study on Parkinson's Disease: Safety and Tolerability of 24-Hour Daily Exposure to Foscarbidopa/Foslevodopa Subcutaneous Infusion by Continuous Subcutaneous Infusion

### Clinical Study B

The objectives of the study were to assess the safety, tolerability, and efficacy of the foscarbidopa/foslevodopa subcutaneous infusion as measured by patient-reported and rater-measured efficacy endpoints. The endpoints included:
percentage of subjects with adverse events (AEs) and serious adverse events during the study,
percentage of subjects with AEs of special interest (AESI) during the study,
percentage of subjects with numeric grade equal to or higher than 5 and percentage of subjects with letter grade equal to or higher than D on the Infusion Site Evaluation Scale at any time during the study,
change in clinical laboratory test data from Baseline to end of study,
change in vital sign measurements from Baseline to end of study,
change in electrocardiograms from Baseline to end of study,
   Secondary Endpoints are change from Baseline to end of study for the following:
average normalized daily “Off” time and “On” times as assessed by the PD Diary,
PD symptoms as assessed by the Movement Disorder Society-Unified Parkinson's Disease Rating Scale (MDS-UPDRS) Parts I-IV (or the UPDRS I V in countries where a validated translation of the MDS-UPDRS is not available),
sleep symptoms as assessed by the PD Sleep Scale-2 (PDSS-2),
quality of life as assessed by the PD Questionnaire-39 items (PDQ-39), and
health-related quality of life as assessed by the EuroQol 5-dimensions questionnaire (EQ-5D-5L).
Another endpoint included PD symptoms as assessed by the Parkinson's KinetiGraph™/Personal KinetiGraph™ (PKG) wearable device (as allowed by local regulations) from Baseline to the Week 26 Visit.

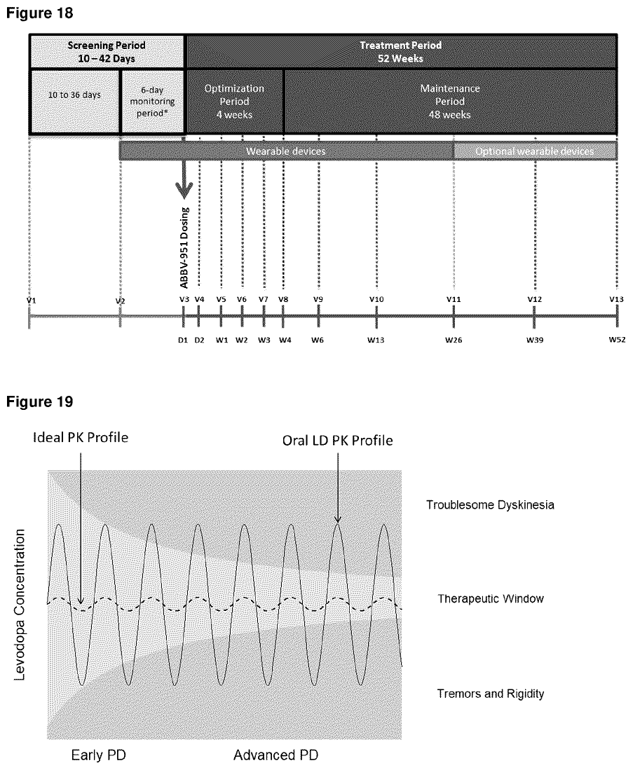

The study was a long-term phase 3 open-label, single-arm study conducted in an outpatient setting for 52 weeks in patients with Parkinson's Disease with 24-hour daily exposure of continuous subcutaneous infusion of the foscarbidopa/foslevodopa subcutaneous infusion. The foscarbidopa/foslevodopa subcutaneous infusion was administered via CSCI for 24 hours daily for 52 weeks The study included about 130 adult subjects with Parkinson's Disease whose motor symptoms are inadequately controlled by oral medications. A schematic of the study is shown in FIG. 18.

### Results

Treatment with the foscarbidopa/foslevodopa subcutaneous infusion resulted in clinically meaningful improvements in “Off” time and “On” time without troublesome dyskinesia. Treatment benefit was observed as early as Week 1 and maintained throughout the 12-month treatment period. At Week 52, per PD diary, the observed mean (SD) reduction from baseline in normalized “Off” time was 3.39 (3.08) hours and increase in “On” time without troublesome dyskinesia was 3.58 (3.03) hours. As shown in Table 32B, the frequent adverse events were infusion site skin events, majority of which were non-serious and mild/moderate in severity. In this open label trial, individualized, 24 h/day foscarbidopa/foslevodopa subcutaneous infusion was generally safe, improved motor complications and morning akinesia, providing a potential efficacious and minimally invasive therapeutic alternative for advanced Parkinson's Disease.

### Subject Disposition, Demographics, and Baseline Characteristics

A total of 244 subjects were enrolled, 7 of them had previously received foscarbidopa/foslevodopa in Phase 1 studies. 103 (42.2%) subjects had prematurely discontinued foscarbidopa/foslevodopa, 115 (47.1%) subjects had completed the 52-week study drug treatment, and 26 (10.7%) subjects were still ongoing with study drug treatment. 145 subjects had at least 180 days of foscarbidopa/foslevodopa exposure and 112 subjects had at least 360 days of foscarbidopa/foslevodopa exposure.

Most analyses are presented by foscarbidopa/foslevodopa dose category. Each subject was individually titrated to attain optimal efficacy and therefore the dose range varied on a continuum. The Low Dose category contains subjects whose modal total daily dose from foscarbidopa/foslevodopa is less than 1800 mg levodopa (LD) based on molecular weight conversion from foslevodopa to LD; while the High Dose category contains subjects with 1800 mg LD or higher modal total daily dose.

TABLE 31A
Subject Disposition

| | foscarbidopa/foslevodopa | | | | | |
|---|---|---|---|---|---|---|
| | Dose Category | | | | | |
| | Low Dose* | | High Dose** | | All Subjects | |
| Enrolled - n(%) | N = 132 | | N = 112 | | N = 244 | |
| Subjects previously | 2 | (1.5) | 5 | (4.5) | 7 | (2.9) |
| exposed to foscarbidopa/ | | | | | | |
| foslevodopa in Phase 1 | | | | | | |
| studies | | | | | | |
| foscarbidopa/foslevodopa | 62 | (47.0) | 53 | (47.3) | 115 | (47.1) |
| completed | | | | | | |
| foscarbidopa/foslevodopa | 17 | (12.9) | 9 | (8.0) | 26 | (10.7) |
| ongoing | | | | | | |
| foscarbidopa/foslevodopa | 53 | (40.2) | 50 | (44.6) | 103 | (42.2) |
| prematurely discontinued | | | | | | |
| Study completed | 67 | (50.8) | 55 | (49.1) | 122 | (50.0) |
| Study ongoing | 22 | (16.7) | 11 | (9.8) | 33 | (13.5) |
| Study prematurely | 43 | (32.6) | 46 | (41.1) | 89 | (36.5) |
| discontinued | | | | | | |

*Low Dose: modal total daily dose from foscarbidopa/foslevodopa <1800 mg levodopa (LD) by molecular weight
**High Dose: modal totally daily dose from foscarbidopa/foslevodopa >=1800 mg LD by molecular weight

TABLE 31B
Demographics and Baseline Characteristics

| | | foscarbidopa/foslevodopa | | | | | |
|---|---|---|---|---|---|---|---|
| | | Dose Category | | | | | |
| | | Low Dose* | | High Dose** | | All Subjects | |
| Parameter | | N = 132 | | N = 112 | | N = 244 | |
| Sex, n (%) | Female | 66 | (50.0) | 32 | (28.6) | 98 | (40.2) |
| | Male | 66 | (50.0) | 80 | (71.4) | 146 | (59.8) |
| Race, n (%) | White | 103 | (78.0) | 104 | (92.9) | 207 | (84.8) |
| | Black or | 0 | | 1 | (0.9) | 1 | (0.4) |
| | African | | | | | | |
| | American | | | | | | |
| | Asian | 27 | (20.5) | 7 | (6.3) | 34 | (13.9) |
| | Other | 2 | (1.6) | 0 | | 2 | (0.8) |
| Age, years | Mean (SD) | 63.6 | (8.89) | 64.3 | (9.53) | 63.9 | (9.18) |
| | Median (min, max) | 65.0 | (34, 83) | 64.0 | (43, 86) | 65.0 | (34, 86) |
| Geographic | North America | 40 | (30.3) | 50 | (44.6) | 90 | (36.9) |
| region, n (%) | | | | | | | |
| | Europe and | 70 | (53.0) | 57 | (50.9) | 127 | (52.0) |
| | Australia | | | | | | |
| | Japan | 22 | (16.7) | 5 | (4.5) | 27 | (11.1) |
| Duration of PD | <10 years | 67 | (50.8) | 64 | (57.1) | 131 | (53.7) |
| since diagnosis | | | | | | | |
| | ≥10 years | 65 | (49.2) | 48 | (42.9) | 113 | (46.3) |
| LED at baseline | n | 131 | | 110 | | 241 | |
| from LD-containing | Mean (SD) | 800.33 | (370.560) | 1380.03 | (636.333) | 1064.93 | (584.809) |
| medication and COMT | Median (min, max) | 798.00 | (100.0, 2906.3) | 1275.00 | (158.8, 3363.0) | 937.50 | (100.0, 3363.0) |
| inhibitor, mg/day | | | | | | | |
| Baseline normalized | n | 127 | | 109 | | 236 | |
| ‘Off’ time (hours) | Mean (SD) | 5.68 | (2.19) | 6.16 | (2.28) | 5.90 | (2.24) |
| | Median (min, max) | 5.8 | (0.0, 10.3) | 6.4 | (0.0, 12.6) | 5.9 | (0.0, 12.6) |

LED = levodopa equivalent.
*Low Dose: modal total daily dose from foscarbidopa/foslevodopa <1800 mg levodopa (LD) by molecular weight
**High Dose: modal totally daily dose from foscarbidopa/foslevodopa >=1800 mg LD bymolecular weight

### Study Periods

### Screening Period

The 10- to 42-day Screening Period consisted of 2 screening visits (V1 and V2) and a 6-day Monitoring Period during which subjects became familiar with the wearable device and the PD Diary. During the Screening Period, subjects were required to demonstrate to be on a stable oral PD medication regimen for at least 30 days prior to commencing foscarbidopa/foslevodopa subcutaneous infusion. Historical data (medical records, etc.) were acceptable documentation for the use of stable medication(s).

Subjects were required to able to safely discontinue any prohibited medications 5 half-lives or 30 days prior to initial foscarbidopa/foslevodopa subcutaneous infusion administration, whichever was longer, with the exception of medications containing levodopa and catechol-O-methyltransferase (COMT)-inhibitors, which were required to be discontinued for at least 12 hours before initiation of foscarbidopa/foslevodopa subcutaneous infusion and for the duration of the Treatment Period. Subjects were required to be consented for the study prior to discontinuing any prohibited medications for the purpose of meeting study eligibility.

Subjects were required to wear the wearable device for 6 consecutive days prior to Visit 3 (i.e., the 6-Day Monitoring Period), in countries where such devices are approved. The PD Diaries were completed for at least 2 consecutive days of the 6-day Monitoring Period, prior to Visit 3 (Day 1).

### Treatment Period

The Treatment Period started with the initiation of foscarbidopa/foslevodopa subcutaneous infusion drug and consisted of 2 parts: a 4-week Optimization Period and a 48-week Maintenance Period.

Optimization Period: during the 4 weeks of the Optimization Period, investigators made necessary adjustments to the subject's foscarbidopa/foslevodopa subcutaneous infusion drug dose until the optimal clinical response for the individual subject was obtained. Additional adjustments were made for the subject's concomitant PD medications (e.g., dopamine-agonists, selective monoamine oxidase B [MAO-B] inhibitors, amantadine, safinamide), including tapering down or even suspending such medications, in accordance with the prescribing information, to achieve the therapeutic approach that, in the investigator's opinion, controlled the subject's symptoms in the most satisfactory way.

Maintenance Period: the Maintenance Period consisted of 48 weeks of a 24-hour daily CSCI of foscarbidopa/foslevodopa subcutaneous infusion at the optimal therapeutic dose. During this 48-week period, subjects maintained a stable regimen of all concomitant medications unless changes were considered medically necessary, in the opinion of the investigator. foscarbidopa/foslevodopa subcutaneous infusion may have been adjusted throughout the study. In the event of dose adjustments for foscarbidopa/foslevodopa subcutaneous infusion or other concomitant medications (including adding or suspending other PD medications) these adjustments were documented in the electronic case report form (eCRF).

On the morning of Day 1 (V3) subjects were in a clinically-defined “Off” state (i.e., no PD medications for at least 12 hours before initiation of foscarbidopa/foslevodopa subcutaneous infusion). After confirmation of eligibility and a brief clinical examination, subjects received an oral loading dose of LD+DDCI followed by the beginning of the subcutaneous infusion of foscarbidopa/foslevodopa subcutaneous infusion at their calculated starting dose.

For each subject, the starting dose level for the continuous infusion rate of foscarbidopa/foslevodopa subcutaneous infusion was calculated according to their baseline dose of oral levodopa (or levodopa equivalent dose [LED]), based on the proposed conversion rates from Tomlinson et al, Error! Reference source not found. guidance from Espay et al, Error! Reference source not found. and a conversion algorithm based on data from Phase 1 studies (See Section Error! Reference source not found.).

Subjects were allowed to resume PD medications that did not contain levodopa or COMT-inhibitors (such as dopamine agonists, MAO-B inhibitors, amantadine, safinamide, etc.) as these concomitant medications were not required to be suspended before starting foscarbidopa/foslevodopa subcutaneous infusion. However, per the investigator's judgment and according to the prescribing information, subjects were allowed to start to taper off concomitant PD medications during the 4 weeks of the Optimization Period.

Subjects were evaluated on the day of, and the day after, initiating foscarbidopa/foslevodopa subcutaneous infusion, and then weekly during the Optimization Period. During each visit, the investigator assessed the subject's PD symptoms by interview and neurologic examination, and may have adjusted the subject's therapeutic regimen by continuing tapering off and/or suspending previous concomitant PD medications, and at the same time increasing, decreasing, or maintaining the foscarbidopa/foslevodopa subcutaneous infusion rate based on the subject's clinical response. If all concomitant PD medications had been tapered off, the dose of foscarbidopa/foslevodopa subcutaneous infusion may have been further adjusted until the optimal clinical response for the individual subject was obtained, as determined by the investigator. Optimal clinical response was defined by maximizing the functional “On” time and minimizing the number of “Off” episodes during the day. This optimization also minimized “On” time with troublesome dyskinesia.

During the Optimization Period, study visits occurred as follows:
Day 1 (V3)
Day 2 (V4)
Week 1 (V5)
Week 2 (V6)
Week 3 (V7)
Week 4 (V8)

The investigator may have performed unscheduled visits to make any needed adjustments within the 4-week Optimization Period.

After Week 4 (V8), all subjects began the Maintenance Period and should have maintained a stable treatment regimen of foscarbidopa/foslevodopa subcutaneous infusion and other concomitant medications, including any PD medications that were still being administered after the Optimization Period. Dose adjustments of medications, including foscarbidopa/foslevodopa subcutaneous infusion, could have been made if considered medically necessary, in the opinion of the investigator. Any changes to medications or the dose of foscarbidopa/foslevodopa subcutaneous infusion were recorded in the eCRF.

During the Maintenance Period, study visits occurred as follows:
Week 6 (V9)
Week 13 (V10)
Week 26 (V11)
Week 39 (V12)
Week 52 (V13) or in case of premature discontinuation.

All visits, apart from the Screening visits (V1 and V2) and V3 (Day 1), were allowed a window of ±3 days.

Unscheduled study visits may have been performed per investigator discretion, if necessary.

TABLE 32A
Change from Baseline in Hours of “On” and “Off” Time Based on PD Diaries

| | | | “On” | |
|---|---|---|---|---|
| | | “On” | Time with | “On” |
| | | Time | Non- | Time with |
| | “Off” | without | Troublesome | Troublesome |
| | Time | Dyskinesia | Dyskinesia | Dyskinesia |
| Study Visit | mean ± SD | | | |
| Baseline | 5.90 ± 2.24 | 6.51 ± 3.35 | 2.63 ± 2.55 | 0.96 ± 1.73 |
| (n = 236) | | | | |
| Week 1 | −1.94 ± 3.68*** | 1.94 ± 4.09*** | −0.05 ± 3.04 | 0.05 ± 1.89 |
| (n = 225) | | | | |
| Week 6 | −2.39 ± 3.60*** | 3.38 ± 4.39*** | −0.58 ± 3.01** | −0.41 ± 1.60*** |
| (n = 205) | | | | |
| Week 13 | −2.42 ± 3.69*** | 4.12 ± 4.35*** | −1.20 ± 3.49*** | −0.50 ± 1.45*** |
| (n = 115) | | | | |
| Week 26 | −3.27 ± 3.24*** | 4.31 ± 4.47*** | −0.66 ± 3.30* | −0.38 ± 1.71* |
| (n = 127) | | | | |
| Week 39 | −3.13 ± 3.18*** | 4.05 ± 4.09*** | −0.59 ± 3.26 | −0.34 ± 1.36* |
| (n = 111) | | | | |
| Week 52 | −3.39 ± 3.08*** | 3.78 ± 4.29*** | −0.21 ± 3.37 | −0.19 ± 1.50 |
| (n = 96) | | | | |

***, **, *indicates change from baseline at P < 0.001, P < 0.01 and P < 0.05. PD, Parkinson's disease.

TABLE 32B
Safety Summary

| | Total, n (%) | |
|---|---|---|
| | (N = 244) | |
| Safety Overview | | |
| Any TEAE | 230 | (94.3) |
| Any severe TEAE | 62 | (25.4) |
| Any serious TEAE | 61 | (25.0) |
| Any TEAE considered associated | 224 | (91.8) |
| with study drug | | |
| Any TEAE leading to premature | 62 | (25.4) |
| study drug discontinuation | | |
| Death^{a} | 3 | (1.2) |
| Most Frequent (≥10%) Treatment- | | |
| Emergent AEs by Preferred Term | | |
| Infusion site erythema | 125 | (51.2) |
| Infusion site nodule | 68 | (27.9) |
| Infusion site cellulitis | 56 | (23.0) |
| Infusion site oedema | 47 | (19.3) |
| Hallucination | 41 | (16.8) |
| Fall | 39 | (16.0) |
| Infusion site pain | 38 | (15.6) |
| Infusion site reaction | 31 | (12.7) |
| Anxiety | 28 | (11.5) |
| Infusion site abscess | 26 | (10.7) |
| Dizziness | 25 | (10.2) |

^{a}none of the events were considered by the investigator as reasonably possibly related to study drug.
TEAE, Treatment-emergent adverse event; AE, adverse event; Coded using MedDRA, Medical Dictionary for Regulatory Activities v 24.0.

### Treatment Arm

Subjects received an oral loading dose on Day 1 (V3) followed by a 24-hour daily subcutaneous infusion of foscarbidopa/foslevodopa subcutaneous infusion. The loading dose was established by the investigator per the subject's oral regimen prior to commencing foscarbidopa/foslevodopa subcutaneous infusion. The starting continuous infusion dose (F1) was calculated taking into consideration the LED and the pharmacokinetic characteristics of foscarbidopa/foslevodopa subcutaneous infusion. The investigator may have adjusted the prescribed infusion rate during visits to reach optimization of motor symptoms control. Subjects could choose their daily continuous infusion dose from 3 preprogrammed flow rates. The investigator determined and programmed the main daily flow rate (F1) within the allowable range (0.17 to 1.04 mL/hr) and may program 2 additional flow rates (F2 and F3) within a ±20% limit from the prescribed main flow rate. F2 was an alternate higher continuous dose, and F3 was an alternate lower continuous dose. If programmed, the alternate flow rates (F2 and F3) were within the allowable range (0.17 to 1.04 mL/hr). If the alternate flow rates (F2 and F3) are not required, they were programmed to “OFF.” Subjects received a System User Manual.

### 4.2 Discussion of Study Design

### Appropriateness of Measurements

Standard statistical, clinical, and laboratory procedures were used in this study. All efficacy measurements were qualified for assessing disease activity in subjects with PD.

### Suitability of Subject Population

The subject population consisted of subjects with PD who report motor complications that are inadequately controlled by oral medications and who experience a minimum of 2.5 hours of “Off” time per day as assessed by PD diaries completed prior to enrollment/Day 1 (V3). Subjects were on a stable oral PD medication regimen for at least 30 days prior to commencing foscarbidopa/foslevodopa subcutaneous infusion.

### Rationale for Selection of Therapeutic Dose Range

CD/LD remains the standard of care for PD. It is, however, recognized that the treatment of PD is highly individualized; both the treatments administered and their doses must be customized, based on the patients' signs and symptoms and their response to medication. In practice, this provides a wide distribution of patient doses, even within clinical trials.

In the LCIG double-blind, double dummy pivotal, the median dose of levodopa was 1013 mg/day and the mean dose was 1117 mg/day for 16-hour daily infusion. These data represent a small subset of subjects where dosing was constrained to maintain the blinding of the study. Therefore, >90% of subjects used less than 2000 mg over a 16-hour treatment period.

In the LCIG open-label safety study in which >300 subjects were enrolled, the dosing was more reflective of clinical practice, where a significant proportion of subjects were able to simplify their therapy by discontinuing concomitant oral medications and maintaining only LCIG during the waking time. From the distribution of the LD doses in this study (Table), approximately 50% of subjects required daily levodopa doses of 1400 mg/day or higher over a 16-hour treatment period.

The foscarbidopa/foslevodopa subcutaneous infusion is a 24-hour/day therapy (i.e., subjects will be exposed 8 more hours—or 50% longer—compared to the LCIG exposure). The results of a PK study comparing LD plasma concentrations between LCIG plus LD/CD tablets and foscarbidopa/foslevodopa subcutaneous infusion in healthy volunteers demonstrated that LD from foscarbidopa/foslevodopa subcutaneous infusion is 8% more bioavailable than enterally-absorbed LD. Based on molecular weight (MW), 100 mg of LD are equivalent to 141 mg of LDP.

Using these data, it was estimated that about 50% of the subjects should be receiving a dose of foscarbidopa/foslevodopa subcutaneous infusion>2800 mg of LDP (equivalent to approximately 2000 mg of levodopa, which is the current maximum recommended dose of LCIG over a 16-hour treatment period), this group is defined as “high dose”.

TABLE 33
LD Dose Distributions

| Distribution | | | | | |
|---|---|---|---|---|---|
| of LD Doses | | | | Estimated Doses over | |
| over 16 hours^{a} | Frequency | | Cumulative | 24 hours | |
| (mg) | (N) | (%) | Percent (%) | LD (mg) | LDP (mg) |
| <500 | 1 | 0.3 | 0.3 | | |
| 500-750 | 12 | 3.8 | 4.1 | | |
| 750-1000 | 29 | 9.1 | 13.2 | | |
| 1000-1250 | 48 | 15.1 | 28.4 | | |
| 1250-1500 | 61 | 19.2 | 47.6 | | |
| 1400 | | | 50 | 2000 | 2800 |
| 1500-1750 | 53 | 16.7 | 64.4 | | |
| 1750-2000 | 47 | 14.8 | 79.2 | | |
| 2000-2250 | 22 | 6.9 | 86.1 | | |
| 2250-2500 | 17 | 5.4 | 91.5 | | |
| 2500-2750 | 13 | 4.1 | 95.6 | | |
| 2750-3000 | 5 | 1.6 | 97.2 | | |
| 3000-3250 | 4 | 1.3 | 98.4 | | |
| 3250-3500 | 3 | 0.9 | 99.4 | | |
| >3500 | 2 | 0.6 | 100.00 | | |
| | Total: 317 | | | | |

Approximately 50% of subjects from the Study required daily levodopa doses of 1400 mg/day or higher over a 16-hour treatment period; considering the additional 8 hours of treatment and the higher bioavailability of foscarbidopa/foslevodopa subcutaneous infusion compared to LCIG, it is estimated that 50% of subjects will require daily levodopa doses of 2000 mg/day or higher over the 24-hour treatment period. Based on molecular weight, 2000 mg of levodopa are equivalent to approximately 2800 mg of LDP.

The distribution of the dose levels of foscarbidopa/foslevodopa subcutaneous infusion was continuously monitored throughout the study with the goal of enrolling approximately one-half of the subjects in the “high dose” group. However, based on an assessment of the dose distribution performed when all subjects reach the end of the Optimization Period, as well as in several other points during the study, the cutoff for the “high dose” group may be revised to be lower or higher than 2000 mg of LD (2800 mg of LDP). This assessment should provide guidance for the analysis of the data and a suitable maximum recommended dose for foscarbidopa/foslevodopa subcutaneous infusion over 24 hours could be proposed.

### 4.3 Loading Dose, Continuous Infusion Rates, Titration and Extra Doses

### Oral Loading Dose

The purpose of the oral loading dose is to allow subjects to achieve symptom control quickly upon initiation of treatment. The loading dose for this study was an oral dose of LD+DDCI corresponding as closely as possible to the subject's habitual first morning dose of LD+DDCI. For those subjects who utilize CD/LD immediate release (IR) for the loading dose, investigators chose from one of 3 doses: 25/100 mg (1 tablet of CD/LD IR 25/100 mg), 37.5/150 mg (1+½ tablets of CD/LD IR), or 50/200 mg (2 tablets of CD/LD IR). Alternatively, LD+DDCI may have been utilized for the loading dose; if so, the amount of DDCI may have varied based on the country of interest; however, the amount of levodopa administered was 100, 150, or 200 mg.

Loading doses were administered only once, at the initiation of foscarbidopa/foslevodopa subcutaneous infusion treatment, unless dosing was discontinued for >8 hours. Temporary disconnection (<1 hour) for daily hygiene or to change empty syringes was not counted as discontinuation and does not require a loading dose upon resuming the infusion.

### Continuous Infusion Rate

Following the oral loading dose, foscarbidopa/foslevodopa subcutaneous infusion was delivered continuously (24 hours daily) via an infusion set connected to a pump. Each subject's initial main continuous infusion rate (F1) was calculated based on the subject's oral LD therapy over the 16-hour treatment period and an algorithm developed following a combination of PK and clinical considerations from Phase 1 studies. A subject's baseline dose of IR LD+DDCI was the starting point for establishing the levodopa equivalent dose (LED) for dose selection (note: the levodopa contained in Stalevo® [CD/LD/entacapone] counts as IR). If a subject is not taking an IR formulation of LD+DDCI, the LED is calculated from the primary levodopa-containing medication. The conversion factors to use in calculating the total LED for dose selection are provided in Table 34 and are based on data from the literature (Tomlinson et al., Mov Dis 2010; Espay et al., Neurol Clinical Practice 2017). The applicable calculation from Table 34 and a subject's baseline CD/LD IR dose should be added together to provide the total starting LED for foscarbidopa/foslevodopa subcutaneous infusion conversion.

TABLE 34
Levodopa Equivalent Dose Range

| Medication | Conversion Factors to Calculate LED | |
|---|---|---|
| Controlled-release | For subjects taking controlled-release levodopa, | |
| levodopa (e.g., | multiply their daily dose of controlled-release | |
| Sinemet CR, | levodopa by 0.75 to obtain their LED. | |
| Madopar CR) | | |
| Extended-release | For subjects taking extended-release levodopa, | |
| levodopa (Rytary) | multiply their total daily dose of Rytary by the | |
| | appropriate conversion ratio in the following | |
| | table to obtain their LED. | |
| | Rytary Dose | Conversion Ratio |
| | Range (mg) | (Rytary to LED) |
| | 0-855 | 0.42 |
| | 856-1755 | 0.48 |
| | 1756-2340 | 0.56 |
| | 2341 or above | 0.67 |

| | |
|---|---|
| Adjustment for | If subjects are taking tolcapone or entacapone, |
| subjects taking | all LED calculated from the levodopa-containing |
| COMT-inhibitors | products must be multiplied by 1.33. |
| such as entacapone | Note: The levodopa dose contained in Stalevo |
| (e.g., Comtan or | counts as IR. |
| Stalevo) or | |
| tolcapone (e.g., | |
| Tasmar) | |

COMT = catechol-O-methyltransferase;
CR = controlled release;
IR = immediate release;
LED = levodopa equivalent dose

Once a subject's daily LED has been determined, the starting infusion rate for foscarbidopa/foslevodopa subcutaneous infusion should be selected from Table 35. The continuous infusion rates below are estimated for the mid-point of the LED interval they refer to and are designed to provide therapeutic levodopa exposure. The infusion rate will be delivered continuously via an infusion set connected to an ambulatory pump.

TABLE 35
foscarbidopa/foslevodopa subcutaneous
infusion starting Infusion Rate

| | foscarbidopa/foslevodopa |
|---|---|
| Daily LED From | subcutaneous infusion |
| Oral Medications | Starting Infusion Rate |
| at Baseline (mg)^{a} | (mL/hr)^{b} |
| <500 | 0.17 |
| 500-599 | 0.20 |
| 600-699 | 0.23 |
| 700-799 | 0.27 |
| 800-899 | 0.30 |
| 900-999 | 0.33 |
| 1000-1099 | 0.37 |
| 1100-1199 | 0.40 |
| 1200-1299 | 0.43 |
| 1300-1399 | 0.47 |
| 1400-1499 | 0.50 |
| 1500-1599 | 0.53 |
| 1600-1699 | 0.57 |
| 1700-1799 | 0.60 |
| 1800-1899 | 0.63 |
| 1900-1999 | 0.67 |
| >2000 | 0.70 |

LED = levodopa equivalent dose
^{a}LED calculated over the 16-hour treatment period
^{b}The suggested infusion rates are calculated for the mid-point of the interval they refer to.

### Titration

The foscarbidopa/foslevodopa subcutaneous infusion main infusion rate (F1) may be adjusted at the investigator's discretion at any time throughout the study to achieve and maintain an optimal therapeutic response for the individual subject, which means maximizing the functional “On” time during the day by minimizing the number and duration of “Off” episodes (bradykinesia) and minimizing “On” time with troublesome dyskinesia. The Infusion rate can be increased or decreased by increments of 0.022 mL/hr, corresponding to approximately 3.75 mg LD/hr (note, the pump displays rates rounded up to the second decimal). Therefore, an increase in main infusion rate of 0.02 mL/hr will result in total increase of approximately 90 mg of LD over the 24-hour period. An increase of 0.04 mL/hr will result in a total increase of approximately 180 mg of LD over the 24-hour period. A decrease of 0.07 mL/hr will result in a total decrease of approximately 270 mg of LD over the 24-hour period, etc.

The Investigator may decide to increase the infusion rate only after assessing the subject for clinical efficacy (achieving optimal therapeutic response defined as above) and safety, via a neurological and physical examination. The safety profile of foscarbidopa/foslevodopa subcutaneous infusion at doses higher than 4000/200 LDP/CDP resulting from infusion rates>0.70 mL/hr, will be assessed throughout the study via neurological examinations and physical assessment (focusing on peripheral neuropathy, weight loss, orthostatic hypotension, and other AESI such as daytime somnolence and hallucinations/psychosis).

### Alternative Continuous Infusion Rates (F2, F3)

Like other therapeutic approaches for PD, which require a personalized titration, and considering the extensive knowledge that advanced patients have about recognizing whether PD symptoms are sufficiently controlled by their therapy, this study provides the subjects the ability to select from alternative infusion rates within a specified range, if allowed by the investigator.

Two additional infusion rates (F2 and F3) may be preprogrammed by the investigator into the pump.

F2 should be an alternative higher continuous dose, and F3 should be an alternative lower continuous dose. The additional infusion rates must be selected within a ±20% limit from the prescribed main continuous infusion rate (F1).

Infusion rates lower than 0.17 mL/hr or higher than 1.04 mL/hr are not allowed.

If during the study the main daily infusion rate (F1) is programmed at 0.17 mL/hr, the alternative lower infusion rate (F3) must be disabled. If at any point during the study the main daily infusion rate (F1) is programmed at 1.04 mL/hr, the alternative higher infusion rate (F2) and the extra dose option (see below) must be disabled.

The starting infusion rate must not be higher than 0.70 mL/hr; adjustments may be done only after clinical assessment (neurological and physical examination).

If the alternate infusion rates (F2 and F3) are not required, the investigator or his/her designee must disable them by program them to “OFF” and the subject will not be able to choose alternative infusion rates. If the investigator enables these options, the subject may select the infusion rate from these 3 preprogrammed choices (F1, F2, or F3) and will be required to record on the dosing diary the time of the day when they choose an alternative infusion rate. The numerical values of the infusion rates can never be modified by the subjects.

### Extra Doses

If the investigator allows it, subjects may also self-administer extra doses of foscarbidopa/foslevodopa subcutaneous infusion during the course of the 24 hours; the ability to self-administer extra doses, the extra dose volume and lockout time (the interval between extra doses) are determined and programmed by the investigator or his/her designee and cannot be modified by the subjects. The investigator can choose the volume of the extra doses from the following options: 0.10, 0.15, 0.20, 0.25, or 0.30 mL (which are equivalent to ˜17, ˜26, ˜34, ˜43, and ˜51 mg of levodopa, respectively). The lockout time is programmable from 1 hour to 24 hours in 15-minute increments; the minimum lockout duration is 60 minutes. If the investigator elects not to allow the use of extra doses, the extra dose should be set to “OFF”.

If the extra dose function is enabled, subjects are required to record on the dosing diary the time of the day when they self-administered an extra dose. Dosing diaries will be reviewed by the study personnel at every study visit.

If the need for extra doses is frequent (greater than 5 times per day), the investigator should consider increasing the main daily infusion rate and increasing the lockout time to limit the number of extra doses per day. If at any time in the study F1 or F2 are set at 1.04 mL/h, the extra dose option should be disabled. Doses of LDP higher than 6000 mg/24 hr (equivalent to 4260 mg/hr of LD) are not allowed, under any circumstances, during the study.

The investigator should consider this upper limit when programming alternative infusion rates and/or volumes and numbers of extra doses. A guidance is provided in Table 36.

TABLE 36
Guidance for Alternative Infusion Rate Limits and Number
of Extra Doses Based on Main Continuous Infusion Rate

| | | | | | | | | Total Max | | |
|---|---|---|---|---|---|---|---|---|---|---|
| | | | | | | | | foscarbidopa/foslevodopa | | |
| F1 Main | | | | | | | | subcutaneous | | |
| Infusion | F2 Upper | F3 Lower | Number of Extra Doses^{a} | | | | | infusion/Day^{a} | | |
| Rate | Limit | Limit | 0.10 | 0.15 | 0.20 | 0.25 | 0.30 | Volume | LDP | LD |
| (mL/hr) | (mL/hr) | (mL/hr) | mL | mL | mL | mL | mL | (mL) | (mg) | (mg) |
| 0.17 | ≤0.20 | OFF | 5 | 5 | 5 | 5 | 5 | 6.4 | 1535.0 | 1089.9 |
| 0.2 | ≤0.24 | ≥0.17 | 5 | 5 | 5 | 5 | 5 | 7.3 | 1742.4 | 1237.1 |
| 0.23 | ≤0.28 | ≥0.18 | 5 | 5 | 5 | 5 | 5 | 8.1 | 1949.8 | 1384.3 |
| 0.27 | ≤0.32 | ≥0.22 | 5 | 5 | 5 | 5 | 5 | 9.3 | 2226.2 | 1580.6 |
| 0.3 | ≤0.36 | ≥0.24 | 5 | 5 | 5 | 5 | 5 | 10.1 | 2433.6 | 1727.9 |
| 0.33 | ≤0.40 | ≥0.26 | 5 | 5 | 5 | 5 | 5 | 11.0 | 2641.0 | 1875.1 |
| 0.37 | ≤0.44 | ≥0.30 | 5 | 5 | 5 | 5 | 5 | 12.2 | 2917.4 | 2071.4 |
| 0.4 | ≤0.48 | ≥0.32 | 5 | 5 | 5 | 5 | 5 | 13.0 | 3124.8 | 2218.6 |
| 0.43 | ≤0.52 | ≥0.34 | 5 | 5 | 5 | 5 | 5 | 13.9 | 3332.2 | 2365.8 |
| 0.47 | ≤0.56 | ≥0.38 | 5 | 5 | 5 | 5 | 5 | 15.0 | 3608.6 | 2562.1 |
| 0.5 | ≤0.60 | ≥0.40 | 5 | 5 | 5 | 5 | 5 | 15.9 | 3816.0 | 2709.4 |
| 0.53 | ≤0.64 | ≥0.42 | 5 | 5 | 5 | 5 | 5 | 16.8 | 4023.4 | 2856.6 |
| 0.57 | ≤0.68 | ≥0.46 | 5 | 5 | 5 | 5 | 5 | 17.9 | 4299.8 | 3052.9 |
| 0.6 | ≤0.72 | ≥0.48 | 5 | 5 | 5 | 5 | 5 | 18.8 | 4507.2 | 3200.1 |
| 0.63 | ≤0.76 | ≥0.50 | 5 | 5 | 5 | 5 | 5 | 19.6 | 4714.6 | 3347.3 |
| 0.67 | ≤0.80 | ≥0.54 | 5 | 5 | 5 | 5 | 5 | 20.8 | 4991.0 | 3543.6 |
| 0.7 | ≤0.84 | ≥0.56 | 5 | 5 | 5 | 5 | 5 | 21.7 | 5198.4 | 3690.9 |
| 0.73 | ≤0.88 | ≥0.58 | 5 | 5 | 5 | 5 | 5 | 22.5 | 5405.8 | 3838.1 |
| 0.77 | ≤0.92 | ≥0.62 | 5 | 5 | 5 | 5 | 5 | 23.7 | 5682.2 | 4034.4 |
| 0.8 | ≤0.96 | ≥0.64 | 5 | 5 | 5 | 5 | 5 | 24.5 | 5889.6 | 4181.6 |
| 0.83 | ≤1.00 | ≥0.66 | 5 | 5 | 5 | 4 | 4 | 25.0 | 5990.4 | 4253.2 |
| 0.86 | ≤1.03 | ≥0.69 | 2 | 1 | 1 | 1 | 1 | 25.0 | 5990.4 | 4253.2 |

| | | | | | | |
|---|---|---|---|---|---|---|
| 0.89 | ≤1.04 | ≥0.71 | OFF | 25.0 | 5990.4 | 4253.2 |
| 0.92 | ≤1.04 | ≥0.74 | OFF | 25.0 | 5990.4 | 4253.2 |
| 0.95 | ≤1.04 | ≥0.76 | OFF | 25.0 | 5990.4 | 4253.2 |
| 0.98 | ≤1.04 | ≥0.78 | OFF | 25.0 | 5990.4 | 4253.2 |
| 1 | ≤1.04 | ≥0.80 | OFF | 25.0 | 5990.4 | 4253.2 |
| 1.04 | OFF | ≥0.83 | OFF | 25.0 | 5990.4 | 4253.2 |

^{a}Assuming the higher alternative infusion rate (F2) option is enabled and set at its upper limit

### Conversion Algorithm

The conversion algorithm was created taking into consideration the bioavailability of LDP, 24-hour exposure, and pharmacokinetic data from foscarbidopa/foslevodopa subcutaneous infusion Phase 1 studies.

In a 4-week clinical study in advanced PD patients (4-week Single-Arm, open-label study of foscarbidopa/foslevodopa subcutaneous infusion in subjects with advanced Parkinson's Disease) an algorithm to convert oral LD to foslevodopa and foscarbidopa was created, based on a combination of PK and clinical considerations. An evaluation of the pattern of dose adjustments from the study and the results of another study (3-part open-label, randomized, 2-period crossover study in healthy older volunteers) confirmed the appropriateness of such algorithm to achieve adequate control of motor symptoms in PD patients. Therefore, the current study will utilize a similar approach to convert oral LD to foslevodopa and foscarbidopa as in the 4-week study.

### Assumptions Used to Generate the Algorithm

Most PD patients are treated with oral PD medications during the waking time; therefore, the calculated LED reflects the dopaminergic need during the average 16-hour treatment period. foscarbidopa/foslevodopa subcutaneous infusion is administered over 24 hours, so 50% more drug is added to account for the additional 8 hours of treatment. Data suggest that the low variability and fluctuations in plasma LD exposure from continuous delivery translate in an average reduction of efficacious daily levodopa doses (Othman A A, et al. J Parkinson's Dis. 2017; 7(2): 275-278.). Additionally, from PK studies comparing LD plasma concentrations between LCIG and foscarbidopa/foslevodopa subcutaneous infusion it appears that foscarbidopa/foslevodopa subcutaneous infusion is more bioavailable than enterally-absorbed levodopa. Finally, a conversion factor of oral levodopa to LDP was determined as 1.41 based on differences in molecular weight.

1 mL of the foscarbidopa/foslevodopa subcutaneous infusion drug product used in this study contains 240 mg of LDP and 12 mg of CDP. Once the total need of LDP per day has been calculated, it is divided by 240 mg to determine the number of milliliters needed per day, and then divided over 24 hours to establish a flow rate. Each suggested infusion rate is calculated for the midpoint of the LED range represented in Table 35.

### Guidance for Enabling Alternative Infusion Rate and Extra Dose Options

The investigator should consider whether to enable alternative infusion rates and extra doses and provide those options to patients, based on his/her clinical judgement of the subject's cognitive performance, compliance, and personality traits.

Allowing an alternative higher infusion rate (F2) could facilitate refining the main continuous infusion rate during the optimization period. Subjects who report troublesome dyskinesia, freezing of gait, or nocturnal akinesia at baseline are often treated with sub-optimal oral daily doses of LD (Cruse, 2018 NPJ Parkinson's Dis; Chang, 2015 PRD). In these cases, programming F1 as suggested in Table 35 could result in a sub-optimal dose of foscarbidopa/foslevodopa subcutaneous infusion. The Investigator could program F2, starting with small increments (e.g. 5-10%), and enable subjects who feel underdosed to switch to the alternative higher infusion rate. Similarly, if subjects are expected to sustain extended physical activity (e.g. sport) or require higher doses of levodopa in a specific time of the day, F2 could be temporarily selected to account for the higher dopaminergic demand. Also, if subjects need to self-administer more than 5 extra doses during the day, they could be instructed to switch to F2 and reduce the use of extra doses, while waiting for an adjustment of the main infusion rate by the Investigator at the next study visit after reviewing the dosing diary. It is important that subjects be reminded to log on the dosing diary any time they self-administer an extra dose or switch the infusion rate to one of the enabled options.

If the investigator prefers not to provide this option or considers the subject not suitable to choose among the provided options, he/she should disable the alternative higher infusion rate by programming it to “OFF”.

Investigators could consider whether to allow subjects to switch to an alternative lower infusion rate (F3) before going to bed, if subjects report no complaints of nocturnal akinesia or nighttime symptoms at baseline or if subjects have known history of impulse control disorders or nocturnal hallucinations (Ricciardi, 2016; Nyholm, 2012; Cruse, 2018). If, between visits during the optimization period, subjects experience sustained dyskinesia, they could be instructed to select the alternative lower infusion rate and record it in their dosing diary, until the investigator adjusts the main parameters at the following study visit. The alternative lower infusion rate F2 may be programmed to be up to 20% lower than F1.

If nocturnal akinesia is present or if bothersome nighttime symptoms exist, investigators should consider maintaining the main infusion rate (F1) consistent for the 24 hours and disable the option for the alternative lower infusion rate (programming it to “OFF”). Similarly, if the investigator prefers not to provide this option or considers the subject not suitable to choose among the provided options, he/she should disable the alternative lower infusion rate by programming it to “OFF”.

### 5 Study Activities

### 5.1 Eligibility Criteria

All subjects were evaluated to ensure that they meet the eligibility criteria at Screening Visits 1 and 2 (V1 and V2) and prior to enrollment on Day 1 (V3).

Demographic and Laboratory Assessments
1. Subject must be an adult male or female, 30 years of age, or older.
2. Subject is willing and able to comply with procedures required in this protocol.
3. Subject does not have a low vitamin B₁₂ level (<200 pg/mL); or does not have low-normal vitamin B₁₂ level (<300 pg/mL) with elevated methylmalonic acid (MMA>0.41 mmol/L) at Screening Visit 1 (V1).
4. Subject has normal cognitive function (Mini-Mental State Examination [MMSE] score of 24 or greater). Subjects with mild cognitive impairment (MMSE score of 19-23 inclusive) may be enrolled if in the investigator's opinion, are able to adhere to all study requirements. Subjects with moderate or severe impairment (MMSE scores of 18 or below) may not be enrolled.
5. Subject is not considered by the investigator to be an unsuitable candidate to receive study agent for any reason.

### Disease Activity

6. Subject must have a diagnosis of levodopa-responsive idiopathic PD.
7. Subject must meet the following disease activity criteria:
   Must be taking a regimen of oral medications for PD that has remained unchanged for at least 30 days before commencing study agent; this regimen must include levodopa-containing formulations such as CD/LD IR (e.g., Sinemet, Madopar), CD/LD-CR (e.g., Sinemet CR), CD/LD extended release (e.g., Rytary), CD/LD/entacapone (e.g., Stalevo).
   Must have a recognizable/identifiable “Off” and “On” state (motor fluctuations) as established through investigator observation and confirmed by PD diary entries recorded during the concordance test performed during the Screening Period.^{b}
8. Must be judged to be inadequately controlled by current therapy in the opinion of the investigator and experience a minimum of 2.5 hours of “Off” time per day as assessed by PD Diary prior to Day 1 (V3). Subjects who have received DBS therapy are eligible for the study, provided they are considered stable, are still levodopa responsive, and meet all other eligibility criteria.
   foscarbidopa/foslevodopa subcutaneous infusion and Study Devices

The investigational product consists of foscarbidopa/foslevodopa subcutaneous infusion (solution for infusion) and study devices e.g., infusion pump, such as Crono PAR Series 3, with infusion set.

TABLE 37
Identity of foscarbidopa/foslevodopa subcutaneous infusion

| foscarbidopa/foslevodopa | | Deliverable Content |
|---|---|---|
| subcutaneous infusion | Dosage Form | per Vial |
| foscarbidopa/foslevodopa | Solution for | 180 mg/3600 mg |
| subcutaneous infusion | infusion | (CDP4′/LDP4′) |
| 12/240 mg/mL (CDP4′/ | | |
| LDP4′) S. INF. 15 mL | | |
| Sodium hydroxide | — | Adjusted to pH 7.4 |
| Water | — | q.s. ad 1.00 mL |

q.s ad = quantum sufficiat ad (a sufficient quantity to make)

Clinical study personnel will receive the System User Manual and System Programming Guide. Subjects will receive the foscarbidopa/foslevodopa subcutaneous infusion System User Manual and Placemat guide for daily use.

Subject dosing will be recorded in a subject dosing diary for 2 consecutive days prior to any study visits as specified in the study activity schedule. The subject will be instructed on how to return all drug containers (even if empty), devices and ancillaries. Clinical study personnel will document compliance.

### Treatments Administered

Subjects will receive an oral loading dose of CD/LD on Day 1 (V3), followed by a 24-hour daily CSCI of foscarbidopa/foslevodopa subcutaneous infusion.

The oral loading dose will be determined by the investigator per therapeutic management prior to infusion. The infusion rate of foscarbidopa/foslevodopa subcutaneous infusion (and therefore the total amount of foscarbidopa/foslevodopa subcutaneous infusion administered) will be calculated based on the subject's levodopa equivalent dose (LED) and an foscarbidopa/foslevodopa subcutaneous infusion conversion algorithm. The initial dose of foscarbidopa/foslevodopa subcutaneous infusion and corresponding flow rate can be adjusted during the Optimization Period to reach the individualized therapeutic dose that most adequately controls the subject's motor symptoms as assessed by the investigator through subject interviews and/or other evaluations of symptom severity. The foscarbidopa/foslevodopa subcutaneous infusion basal infusion rate can also be adjusted throughout the study at the discretion of the investigator. The investigator may preprogram 3 flow rates into the pump, 1 that corresponds to the main prescribed daily flow rate (F1, which can range between 0.17 and 1.04 mL/hr) and 2 additional flow rates (F2 and F3) that are programmable within a ±20% limit from the prescribed main flow rate and always within the allowable range (0.17 to 1.04 mL/hr); F2 should be an alternate higher continuous dose and F3 should be an alternate lower continuous dose. If the alternate flow rates (F2 and F3) are not required, the investigator or his/her designee must program them to “OFF.” Thus, subjects might have the freedom to choose among the preprogrammed flow rates or to self-administer extra doses of foscarbidopa/foslevodopa subcutaneous infusion during the day, per the investigator's judgment. The pump will be programmed by the site per instructions in the System Programming Guide.

During the Treatment Period, the foscarbidopa/foslevodopa subcutaneous infusion will be self-administered by the subject using study devices (i.e., infusion set connected to an infusion pump) indicated for subcutaneous delivery. The drug delivery system will need to be loaded with the investigational drug at the same time every day for subjects who require 1 vial per day (every 24 hours), or at the same times of the day (every 12 hours) for subjects who require 2 vials per day. To prepare the drug delivery system, reference the System User Manual instructions: foscarbidopa/foslevodopa subcutaneous infusion will be loaded into a syringe that will then be placed into the pump. The infusion site will be prepared. The infusion set will be attached to the syringe, primed, and then attached to the subject. The daily dose will be initiated by starting the pre-programmed pump. When vial changes are performed, depending on how long the infusion set has already been in use, subjects may need to either: a) obtain all new components or b) obtain all new components except for a new infusion set. For the latter, they can just temporarily disconnect the infusion set from the subject, transfer the other end from the old syringe to the newly prepared syringe, prime and then re-attach it to the subject.

The infusion set and the infusion site (area of the skin where the subcutaneous cannula is inserted) can be left unchanged for up to 3 days when the drug is infused continuously. This includes maintaining the same infusion set when changing empty syringes or using a new vial (for those who require two vials per day). However, a new vial adapter and a new syringe are required any time a new vial is used. The infusion set should be changed and the infusion site rotated every 3 days unless a more frequent rotation schedule is medically indicated. Anytime that the drug delivery is suspended for longer than 1 hour, subjects should use a new vial, new syringe, new vial adapter, and new entire infusion set before resuming foscarbidopa/foslevodopa subcutaneous infusion administration. No action is needed if the infusion pump is disconnected and foscarbidopa/foslevodopa subcutaneous infusion is not administered for less than 1 hour (such as when changing empty syringes). The Investigator will select the length of the infusion set cannula among those provided. If the infusion pump is disconnected and foscarbidopa/foslevodopa subcutaneous infusion is not administered for 1 hour or more, subjects should be instructed to do the following before resuming foscarbidopa/foslevodopa subcutaneous infusion administration:

TABLE 38

| | | | Use new | | |
|---|---|---|---|---|---|
| | | | vial/new | | |
| Duration | | Use new | vial | Rotate | Self- |
| of drug | | entire | adapter/new | infusion | administer |
| suspension | | infusion set | syringe | site | oral dose |
| 1 to <3 | hours | X | X | X | None |
| 3 to ≤8 | hours | X | X | X | 100 mg |
| | | | | | LD + DDCI^{a} |
| >8 | hours | X | X | X | Loading dose^{b} |

^{a}e.g., 25/100 mg CD/LD IR.
^{b}Same dose as that taken prior to commencing foscarbidopa/foslevodopa subcutaneous infusion (e.g., LD + DDCI: 25/100 mg, 37.5/150 mg or 50/200 mg).

The infusion should be administered to a site at least 5 centimeters (or 2 inches) away from the site of application of ECG electrodes, and at least 5 centimeters (or 2 inches) away from the umbilicus. Infusions should not be administered to areas of scarred or hardened tissue or stretch marks, to skin folds or creases where the body naturally bends a great deal, or to areas where clothing might cause irritation (e.g., near the beltline).

In the event of an infusion pump malfunction or other event that temporarily prevents delivery of foscarbidopa/foslevodopa subcutaneous infusion, the time period during which foscarbidopa/foslevodopa subcutaneous infusion was not delivered will be recorded by the site, in the electronic data capture system. The subject should call the clinical study personnel, so that they may attempt to resume the foscarbidopa/foslevodopa subcutaneous infusion. The time that the infusion stopped and/or malfunction will be recorded to the minute if known (otherwise, an estimated time will be recorded). If the infusion is resumed, the restart time will be recorded to the minute. If the foscarbidopa/foslevodopa subcutaneous infusion is not able to be resumed or if the investigator is concerned that the subject may not be able to successfully complete the foscarbidopa/foslevodopa subcutaneous infusion requirements, the subject may be discontinued from the study; discontinued subjects are not planned to be replaced.

### Selection and Timing of Dose for Each Subject

A CSCI of foscarbidopa/foslevodopa subcutaneous infusion will be delivered over 24 hours daily for up to 52 weeks via an infusion set connected to a pump. Each subject's main continuous infusion rate (F1) will be initially calculated based on the subject's daily LED and an algorithm developed following the analysis of pharmacokinetic data from Phase 1 studies. The dose should be adjusted based on the clinical response for the individual subject, which means maximizing the functional “On” time during the day by minimizing the number and duration of “Off” episodes (bradykinesia) and minimizing “On” time with disabling dyskinesia.

### Foscarbidopa/Foslevodopa Subcutaneous Infusion Accountability

Subjects will be instructed to return used drug vials with vial adapters attached within the original vials carton; subjects will also be instructed to return unused vials in the original vial carton. Used syringes, infusion sets (inserter and infusion set tubing) will be collected into a disposal container. Unused syringes, infusion sets (inserter and infusion set tubing) and vial adapters will be returned by the subject to the site at the end of the study.

The investigator or his representative will verify that foscarbidopa/foslevodopa subcutaneous infusion supplies are received intact and in the correct amounts. This will be documented by signing and dating a proof of receipt or similar document. A current (running) and accurate inventory of foscarbidopa/foslevodopa subcutaneous infusion will be maintained in the IRT system. For those subjects who are allowed to benefit from a direct to patient shipment of the foscarbidopa/foslevodopa subcutaneous infusion and foscarbidopa/foslevodopa subcutaneous infusion delivery system and its accessories, the third party vendor contracted by the Sponsor may be responsible of those steps described above.

Site personnel will review returned foscarbidopa/foslevodopa subcutaneous infusion kits, and empty foscarbidopa/foslevodopa subcutaneous infusion packaging to verify compliance. Each site will be responsible for maintaining drug accountability records including product description, manufacturer, and lot numbers for all non-investigational products (e.g., rescue therapy with CD/LD IR) dispensed by the site.

### 5.9 Protocol Deviations

The investigator is responsible for complying with all protocol requirements, written instructions and applicable laws regarding protocol deviations. Protocol deviations are prohibited except when necessary to eliminate an immediate hazard to study subjects.

### 6.0 Efficacy:

At Week 52, per PD diary, the observed mean (SD) reduction from baseline in normalized “Off” time was 3.39 (3.08) hours and increase in “On” time without troublesome dyskinesia was 3.58 (3.03) hours. “On” time without troublesome dyskinesia is the sum of “On” time without dyskinesia and “On” time with non-troublesome dyskinesia. These results are shown in FIG. 21.

### Example 9: Sleep and Efficacy Endpoint Correlations after 6-Months of Subcutaneous Foscarbidopa/Foslevodopa Subcutaneous Infusion in Parkinson's Disease

A post hoc analysis of 6-month interim results from the phase 3 study described in Example 8, Clinical Study B, were evaluated for correlations between sleep, motor complications, motor experiences of daily living, and quality of life (QoL).

Spearman's rank correlation coefficients were provided for measures of sleep (Parkinson's Disease Sleep Scale [PDSS-2]), motor complications, motor experiences of daily living (MDS-UPDRS part II), and QoL (PDQ-39).

Results: Patients improved in assessed outcomes at 6 months (Table 39). Baseline PDSS-2 scores did not correlate with baseline “Off” time, weakly positively correlated with baseline MDS-UPDRS part II scores (P<0.001), and moderately positively correlated with baseline PDQ-39 scores (P<0.001). Improvement from baseline in PDSS-2 scores weakly positively correlated with change from baseline in “Off” time (P<0.001), MDS-UPDRS part II scores (P<0.05), PDQ-39 scores (P<0.001), and motor state at awakening for patients who awoke in “On” time without dyskinesia at baseline, and weakly negatively correlated with “On” time without troublesome dyskinesia (Table 39). foscarbidopa/foslevodopa subcutaneous infusion was well tolerated.

Conclusion: Beneficial effects of foscarbidopa/foslevodopa subcutaneous infusion on sleep in patients with PD are correlated with improvements in “Off” time, “On” time, motor experiences of daily living, and QoL.

TABLE 39
Clinical Characteristics at Baseline and Month 6 (N = 223)

| | | | Change From | |
|---|---|---|---|---|
| Characteristic | Baseline | | Baseline | |
| Daily “Off” time, hours^{a} | 5.9 | (2.3) | −2.9 | (3.2)*** |
| Daily “On” time without | 6.5 | (3.3) | 4.0 | (4.4)*** |
| dyskinesia, hours^{a} | | | | |
| Daily “On” time with non- | 2.7 | (2.6) | −0.8 | (3.4)* |
| troublesome dyskinesia, hours^{a} | | | | |
| Daily “On” time without | 9.2 | (2.4) | 3.2 | (3.2)*** |
| troublesome dyskinesia, hours^{a} | | | | |
| Daily “On” time with troublesome | 0.9 | (1.7) | −0.3 | (1.5) |
| dyskinesia, hours^{a} | | | | |
| PDSS-2 total score | 20.5 | (9.7) | −5.7 | (11.2)*** |
| MDS-UPDRS part II | 15.8 | (7.5) | −3.2 | (6.8)*** |
| PDQ-39 summary index | 34.3 | (14.9) | −7.2 | (11.4)*** |

| Percent of Patients Awakening | | Observed at |
|---|---|---|
| with the Below Motor State | Baseline | Month 6 |
| “Off” time | 77.8 | 20.8 |
| “On” time without dyskinesia | 18.3 | 68.8 |
| “On” time with non-troublesome dyskinesia | 3.3 | 9.1 |
| “On” time with troublesome dyskinesia | 0.7 | 1.3 |

Data are mean (SD) unless otherwise specified.
*P < .05;
**P < .01;
***P < 0.001.
^{a}Normalized to a 16-hour waking day.
MDS-UPDRS, Movement Disorders Society Unified Parkinson's Disease Rating Scale; PDSS-2, Parkinson's Disease Sleep Scale-2; PDQ-39, 39-item Parkinson's Disease Questionnaire.

TABLE 39A
Spearman's Rank Correlation Between Sleep
and Efficacy Endpoints Correlation Coefficient

| | Spearman's Rank | |
|---|---|---|
| | Correlation Coefficient | |
| | | Change From |
| | | Baseline at |
| Variables | Baseline | Month 6 |
| PDSS-2 and “Off” time | 0.069 | 0.388*** |
| PDSS-2 and “On” time without | −0.130 | −0.270* |
| troublesome dyskinesia | | |
| PDSS-2 and “On” time without | −0.063 | −0.193 |
| dyskinesia | | |
| PDSS-2 and MDS-UPDRS II | 0.281*** | 0.212* |
| PDSS-2 and PDQ-39 summary index | 0.438*** | 0.348*** |
| PDSS-2 and motor state at awakening | −0.096 | NA |
| Patients who awoke in “Off” | NA | −0.122 |
| at baseline | | |
| Patients who awoke in “On” | NA | NA |
| with troublesome dyskinesia at | | |
| baseline^{a} | | |
| Patients who awoke in “On” | NA | NA |
| with non-troublesome dyskinesia | | |
| at baseline^{b} | | |
| Patients who awoke in “On” | NA | 0.207 |
| without dyskinesia at baseline | | |

*P < .05;
***P < 0.001.
^{a}NA as only 2 patients with “On” with troublesome dyskinesia at baseline and “On” without dyskinesia at Month 6.
^{b}NA as 0 patients with “On” with non-troublesome dyskinesia at baseline and other motor state at awakening at Month 6.
MDS-UPDRS, Movement Disorders Society Unified Parkinson's Disease Rating Scale; NA, not assessed; PDSS-2, Parkinson's Disease Sleep Scale-2; PDQ-39, 39-item Parkinson's Disease Questionnaire.

### Example 10. Pharmacokinetics of 24-Hour Levodopa and Carbidopa Prodrugs Continuous Subcutaneous Infusion Treatment of Parkinson's Disease in Healthy Human Volunteers

This study was designed to characterize the pharmacokinetics of aqueous pharmaceutical composition comprising a combination of levodopa 4′-monophosphate and carbidopa 4′-monophosphate following continuous subcutaneous infusion to the abdomen.

### Methods

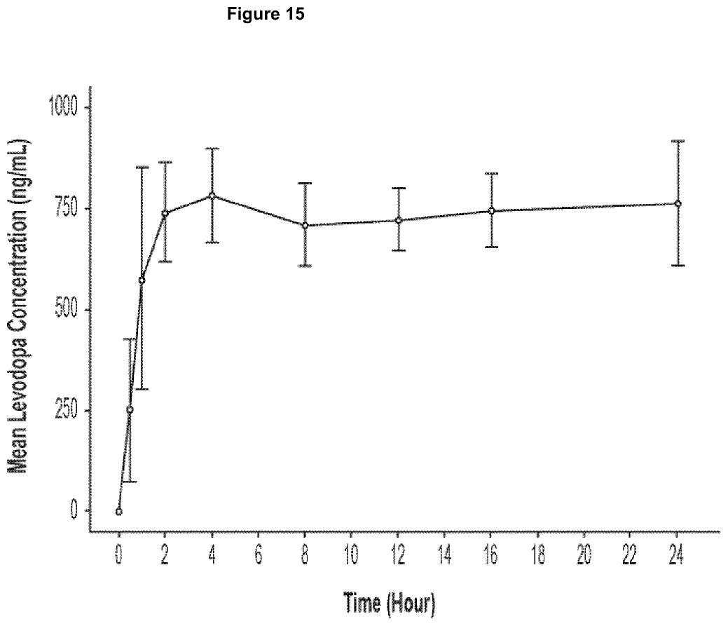

The pharmaceutical composition prodrug was administered subcutaneously to 8 healthy volunteers (45-75 years) for 24 hours in an open label study. The dosing consisted of 100 mg levodopa phosphate loading dose followed by a continuous steady infusion of 850 mg levodopa phosphate over 24-hour period. All the infusions of the pharmaceutical composition were administered to the subcutaneous space in the abdomen. During and following infusion of the pharmaceutical composition, serial plasma samples were collected to assay for levodopa and carbidopa. Levodopa and carbidopa pharmacokinetic data from a previous Duopa phase 1 study (Nyholm, D., et al. AAPS Journal 2013; 15-2: 316-329) was used to compare pharmacokinetic data between the present pharmaceutical composition and Duopa. Safety and tolerability including local adverse events (AEs) related to the subcutaneous infusion site were assessed throughout the study. Following administration of the pharmaceutical composition, levodopa mean pharmacokinetic profile over initial 16 hours is similar to previous Duodopa phase 1 study (Nyholm, D., et al. AAPS Journal 2013; 15-2: 316-329) in Parkinson's Disease patients (FIGS. 15 and 16).

### Results

Following the study, the results were analyzed. Five subjects reported at least one adverse event. Mild infusion site reactions and injection site irritation were observed in a few subjects. All adverse events were transient and did not cause discontinuation from the study and there was no nodule formation at the infusion site.

The pharmaceutical composition was able to provide stable levodopa and carbidopa exposures over 24 hours via the subcutaneous route of delivery with very low fluctuation in levodopa concentration level. The pharmaceutical composition had a favorable safety profile.

### Example 11: Subcutaneous Foscarbidopa/Foslevodopa Subcutaneous Infusion in Parkinson's Disease: Subgroup Analysis from a 6-Month Interim Phase 3 Dataset

Introduction: foslevodopa/foscarbidopa is a soluble formulation of levodopa and carbidopa prodrugs delivered by continuous subcutaneous infusion. Six-month interim results of a phase 3 trial of foscarbidopa/foslevodopa subcutaneous infusion in Parkinson's disease (PD) (Example 8, clinical study) demonstrated significant improvements in “Off” time, “On” time, motor experiences of daily living, and quality of life (QoL). Analyses of these interim data by age, PD duration, and “Off” time at baseline are reported.

Methods: This is an ongoing, open-label, single-arm study assessing the safety and tolerability of long-term foslevodopa/foscarbidopa exposure (NCT03781167). Patients with PD whose motor symptoms were inadequately controlled by their current treatment are receiving a 24-hour/daily optimizeddose of foscarbidopa/foslevodopa subcutaneous infusion for 52 weeks.

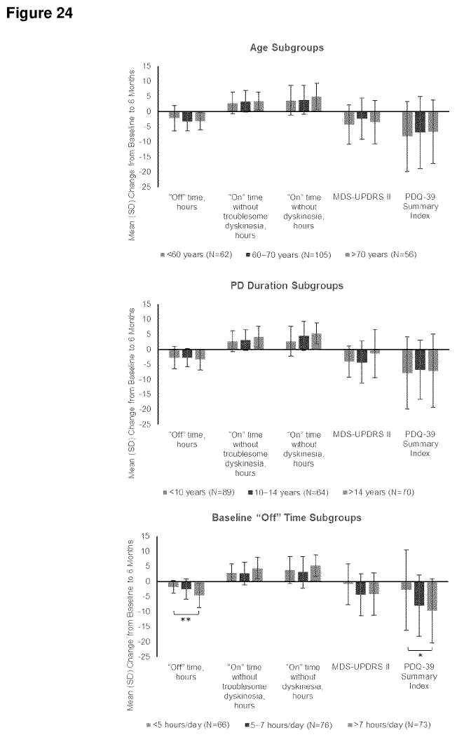

Results: Table 40 reports baseline demographics and characteristics of the 223 patients. Patient age and disease duration had no significant effect on change from baseline to month 6 in “Off” time, “On” time, motor experiences of daily living (MDS-UPDRS part II), and QoL (PDQ-39 Summary Index Total Score) (FIG. 24). In contrast, higher “Off” time at baseline was associated with significantly greater improvements in absolute reduction of “Off” time (P<0.01), and greater improvements in PDQ-39 Summary Index Score (P<0.05) at 6 months (FIG. 24). Safety was generally consistent among groups. Age had the greatest impact on event frequency (Table 41).

Conclusion: This 6-month interim analysis demonstrated that beneficial effects of foslevodopa/foscarbidopa in PD are not affected by age or disease duration. Patients with more severe motor fluctuations at baseline showed the greatest absolute improvements in “Off” time and QoL.

TABLE 40
Baseline Demographics and Characteristics

| | <60 years | | 60-70 years | | >70 years | |
|---|---|---|---|---|---|---|
| Age | (n = 62) | | (n = 105) | | (n = 56) | |
| Age, years | 51.9 | (5.4) | 64.9 | (3.0) | 74.8 | (3.6) |
| Male, n (%) | 47 | (75.8) | 60 | (57.1) | 27 | (48.2) |
| PD duration, years | 10.3 | (5.3) | 12.9 | (4.9) | 13.2 | (5.2) |
| “Off” time, hours | 5.8 | (2.6) | 6.4 | (2.5) | 5.7 | (2.2) |
| MDS-UPDRS II | 13.8 | (5.6) | 15.0 | (7.5) | 19.5 | (8.1) |
| PDQ-39 Summary | 32.0 | (15.4) | 33.9 | (15.0) | 37.6 | (13.8) |
| Index | | | | | | |
| | <10 years | | 10-14 years | | >14 years | |
| PD duration | (n = 89) | | (n = 64) | | (n = 70) | |
| Age, years | 61.2 | (10.5) | 63.4 | (8.3) | 67.3 | (7.4) |
| Male, n (%) | 51 | (57.3) | 43 | (67.2) | 40 | (57.1) |
| PD duration, years | 7.5 | (1.8) | 12.1 | (1.3) | 18.4 | (3.5) |
| “Off” time, hours | 6.2 | (2.6) | 6.1 | (2.4) | 5.8 | (2.5) |
| MDS-UPDRS II | 14.6 | (7.6) | 15.4 | (7.9) | 17.6 | (6.8) |
| PDQ-39 Summary | 33.1 | (14.1) | 33.4 | (15.3) | 36.5 | (15.4) |
| Index | | | | | | |
| | <5 hours/day | | 5-7 hours/day | | >7 hours/day | |
| “Off” time^{a} | (n = 66) | | (n = 76) | | (n = 73) | |
| Age, years | 62.9 | (9.9) | 64.3 | (8.7) | 63.3 | (8.7) |
| Male, n (%) | 40 | (60.6) | 50 | (65.8) | 39 | (53.4) |
| PD duration, years | 12.3 | (5.3) | 12.2 | (4.9) | 12.2 | (5.6) |
| “Off” time, hours | 3.2 | (1.3) | 6.0 | (0.7) | 8.7 | (1.3) |
| MDS-UPDRS II | 15.3 | (8.4) | 16.1 | (6.7) | 15.8 | (7.6) |
| PDQ-39 Summary | 33.2 | (16.7) | 33.9 | (14.1) | 35.8 | (14.2) |
| Index | | | | | | |

Data are mean (SD) unless otherwise specified.
^{a}N = 215.
MDS-UPDRS II, Movement Disorder Society Unified Parkinson's Disease Rating Scale part II; PD, Parkinson's disease; PDQ-39, 39-item Parkinson's Disease Questionnaire.

TABLE 41
Safety Overview

| | <60 years | 60-70 years | >70 years |
|---|---|---|---|
| Age | (n = 62) | (n = 105) | (n = 56) |
| Any TEAE | 60 (96.8) | 96 (91.4) | 50 (89.3) |
| Any severe TEAE | 13 (21.0) | 24 (22.9) | 19 (33.9) |
| Any serious TEAE | 10 (16.1) | 23 (21.9) | 20 (35.7) |
| Any TEAE that are considered | 59 (95.2) | 95 (90.5) | 47 (83.9) |
| associated with foscarbidopa/ | | | |
| foslevodopa subcutaneous | | | |
| infusion ^{a} | | | |
| Any TEAE leading to premature | 12 (19.4) | 26 (24.8) | 16 (28.6) |
| discontinuation of | | | |
| foscarbidopa/foslevodopa | | | |
| subcutaneous infusion | | | |
| Any TEAE leading to death | 0 | 2 (1.9) | 1 (1.8) |
| | <10 years | 10-14 years | >14 years |
| PD duration | (n = 89) | (n = 64) | (n = 70) |
| Any TEAE | 83 (93.3) | 59 (92.2) | 64 (91.4) |
| Any severe TEAE | 21 (23.6) | 17 (26.6) | 18 (25.7) |
| Any serious TEAE | 18 (20.2) | 12 (18.8) | 23 (32.9) |
| Any TEAE that are considered | 82 (92.1) | 58 (90.6) | 61 (87.1) |
| associated with foscarbidopa/ | | | |
| foslevodopa subcutaneous | | | |
| infusion ^{a} | | | |
| Any TEAE leading to premature | 24 (27.0) | 15 (23.4) | 15 (21.4) |
| discontinuation of | | | |
| foscarbidopa/foslevodopa | | | |
| subcutaneous infusion | | | |
| Any TEAE leading to death | 1 (1.1) | 1 (1.6) | 1 (1.4) |
| | <5 hours/day | 5-7 hours/day | >7 hours/day |
| “Off” time^{b} | (n = 66) | (n = 76) | (n = 73) |
| Any TEAE | 60 (90.9) | 70 (92.1) | 68 (93.2) |
| Any severe TEAE | 15 (22.7) | 18 (23.7) | 21 (28.8) |
| Any serious TEAE | 18 (27.3) | 14 (18.4) | 19 (26.0) |
| Any TEAE that are considered | 57 (86.4) | 68 (89.5) | 68 (93.2) |
| associated with foscarbidopa/ | | | |
| foslevodopa subcutaneous | | | |
| infusion ^{a} | | | |
| Any TEAE leading to premature | 17 (25.8) | 16 (21.1) | 19 (26.0) |
| discontinuation of | | | |
| foscarbidopa/foslevodopa | | | |
| subcutaneous infusion | | | |
| Any TEAE leading to death | 1 (1.5) | 1 (1.3) | 1 (1.4) |

Data are n (%).
^{a} As assessed by the investigator.
^{b}N = 215
PD, Parkinson's disease; TEAE, treatment-emergent adverse event.

### Example 12: Impact of Foslevodopa/Foscarbidopa Treatment on Key Clinical and Humanistic Outcomes in Patients with Advanced Parkinson's Disease: Post-Hoc Responder Analysis of Two Phase 3 Clinical Trials

Objective: To evaluate among patients with advanced Parkinson's disease (aPD) the impact of foslevodopa and foscarbidopa on clinical and humanistic outcomes using minimal clinically important difference (MCID) vs. levodopa and carbidopa immediate release oral tablets (LCIR) in an RCT and within-patient change vs. baseline in a single-arm study.

Background: Results from Phase 3 clinical trials have established foslevodopa and foscarbidopa greater efficacy and sustained improvement in patient outcomes. Responder analysis may facilitate interpretations of clinically meaningful treatment effects.

Methods: Post-hoc analyses of data from a 12-week (wk) RCT trial of foslevodopa and foscarbidopa vs LCIR and a 52-wk single-arm trial of foslevodopa and foscarbidopa. Responders were identified relative to baseline for ON without troublesome dyskinesia (ONwoTD) and OFF-time using a threshold of ≥1-hr (≥3-hr for robust responders) increase and decrease, respectively. Established MCIDs were used to evaluate responders for MDS-UPDRS Parts I-IV, quality of life (QoL; PDQ-39 and EQ-5D-5L) and sleep disturbances (PDSS-2). Adjusted general linear models compared responder proportions between foslevodopa and foscarbidopa (n=46) vs. LCIR (n=60) at wk12, and vs. baseline for foslevodopa and foscarbidopa at wk 13 (n=148) and 52 (n=75).

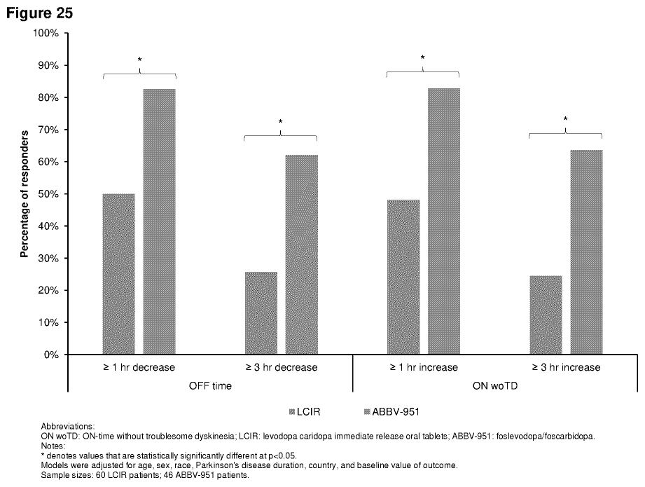

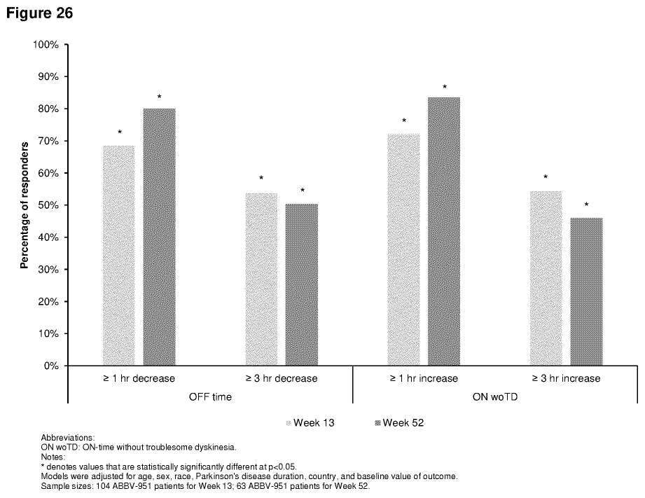

Results: After 12 wk of treatment, significantly higher proportions of foslevodopa and foscarbidopa vs. LCIR patients had ≥1-hr (82.6% vs. 50.0%) and ≥3-hour (62.1% vs. 25.7%) reductions in OFF, and ≥1-hr (82.9% vs. 48.1%) and ≥3-hr (63.6% vs. 24.5%) increases in ONwoTD (all p<0.001) (FIG. 25). For within-patient change vs. baseline of OFF and ONwoTD, foslevodopa and foscarbidopa demonstrated significant immediate response at wk 13, which was maintained up to wk 52 (FIG. 26). Response rates were significantly higher at wk 12 for foslevodopa and foscarbidopa vs. LCIR for MDS-UPDRS Part II (47.0% vs. 21.4%) and IV (60.7% vs. 39.5%), and PDSS-2 (71.5% vs. 42.6%) (all p<0.05). Similar findings were observed for foslevodopa and foscarbidopa in demonstrated immediate and sustained response (up to wk 52) on activities of daily living (ADL), motor complications, QoL, and sleep disturbances. (Table 42).

Conclusions: Greater achievement of clinically meaningful improvement in OFF, ONwoTD, ADL and sleep disturbances were observed for foslevodopa and foscarbidopa vs. LCIR. These clinically meaningful improvements were immediate and consistently sustained up to week 52.

TABLE 42
Percentage of responders (achieving minimal clinically
important difference) for patient-reported outcomes for
foslevodopa and foscarbidopa at week 13 and week 52

| | Week 13 | | | Week 52 | | |
|---|---|---|---|---|---|---|
| | n | Mean | 95% CI | n | Mean | 95% CI |
| MDS-UPDRS | | | | | | |
| Part I | 147 | 40.6% | [33.6%, | 75 | 31.3% | [22.2%, |
| | | | 47.5%] | | | 40.5%] |
| Part II | 147 | 44.5% | [37.3%, | 75 | 51.1% | [40.5%, |
| | | | 52.8%] | | | 61.6%] |
| Part III | 136 | 35.6% | [28.5%, | 75 | 34.4% | [24.3%, |
| | | | 42.8%] | | | 44.5%] |
| Part IV | 147 | 81.5% | [75.5%, | 75 | 86.8% | [79.4%, |
| | | | 87.4%] | | | 94.2%] |
| Sleep quality | | | | | | |
| PDSS-2 | 148 | 63.4% | [56.9%, | 74 | 68.0% | [60.2%, |
| | | | 70.0%] | | | 75.8%] |
| Quality of life | | | | | | |
| PDQ-39 | 148 | 62.7% | [55.4%, | 74 | 52.9% | [41.7%, |
| | | | 70.0%] | | | 64.1%] |
| EQ-5D-5L | 123 | 37.4% | [30.1%, | 69 | 35.3% | [25.3%, |
| | | | 44.7%] | | | 45.3%] |

MDS-UPDRS: Movement Disorder Society - Unified Parkinsons's Disease Rating Scale;
PDSS-2: Parkinson's Disease Sleep Scale 2;
EQ-5D-5L: European Quality of Life Five Dimension - Five Levels
Notes:
MCID thresholds: MDS-UPDRS Part 1: <−2.64; Part II: <−3.05; Part III: <−3.25; Part IV: <−0.9; <−4.72; PDQ−39: <−4.72; Sleep quality: <−3.44.

### Example 13: Improvement in Motor-State Stability Throughout the Day and Rapid Onset to Good ON Time in Patients with aPD Treated with Foslevodopa and Foscarbidopa Vs. LCIR

Title: Improvement in motor-state stability throughout the day and rapid onset to good ON time in patients with aPD treated with foslevodopa and foscarbidopa vs. LCIR Objective: To evaluate time to ON without troublesome dyskinesia (“good ON”) and patterns of motor-state stability throughout the waking day in patients with advanced Parkinson's disease (aPD) treated with foslevodopa and foscarbidopa vs. levodopa and carbidopa immediate release oral tablets (LCIR).

Background: The 24-hour subcutaneous infusion of foslevodopa and foscarbidopa enables continuous dopaminergic stimulation and more stable levodopa plasma levels. The temporal patterns of motor-symptom improvements throughout the day have not been established for foslevodopa and foscarbidopa.

Methods: Post-hoc analyses of patient PD diary data from a phase 3 trial of foslevodopa and foscarbidopa vs. LCIR. Diary data recorded motor-states (OFF, good ON, ON with troublesome dyskinesia, asleep) at 30-minute (min) intervals over 3 days (normalized to 16-hour waking days) at baseline and week 12. Adjusted heteroskedasticity-robust linear regression models assessed changes from baseline to week 12, and between-treatment groups for time to good ON after waking and motor states throughout the day at 30-min and 4-hr intervals, and mean number of daily motor-state transitions.

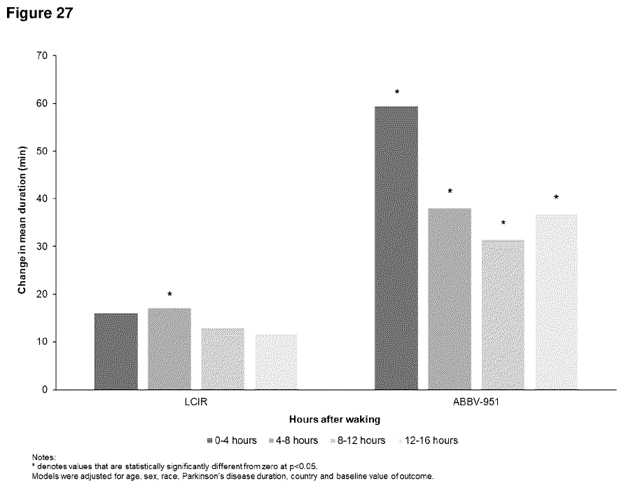

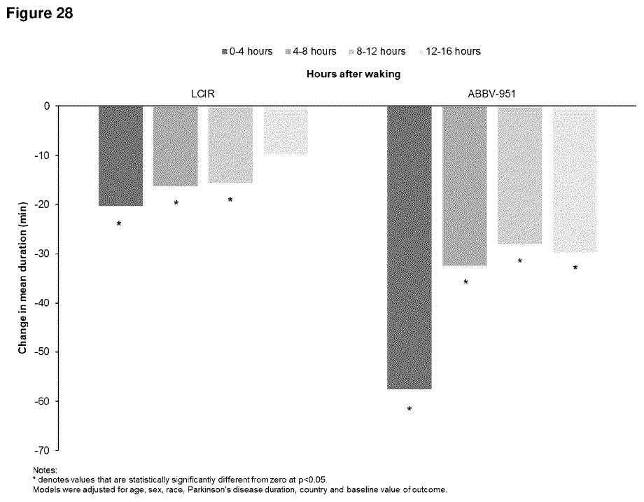

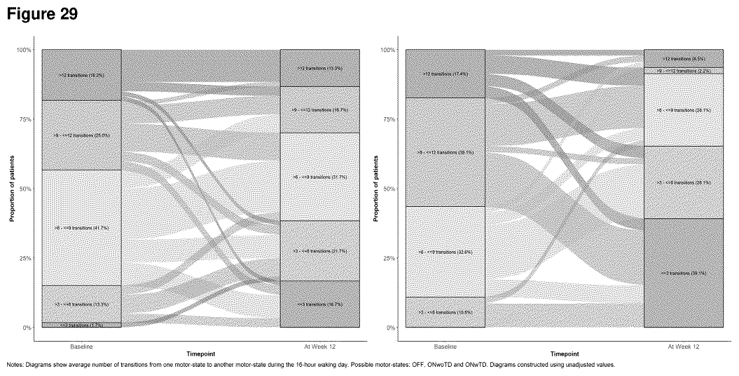

Results: Complete diary data were available for 46 foslevodopa and foscarbidopa and 60 LCIR patients. After 12 weeks of treatment, foslevodopa and foscarbidopa patients reached good ON faster (means: 28.9 vs. 82.9 min; p=0.004) and nearly twice more frequently within 30 minutes after waking (84.7% vs. 47.6%; p<0.001) vs. LCIR. Nearly 4 times fewer (13.8% vs. 57.8%; p<0.001) foslevodopa and foscarbidopa patients reported OFF within 30 min after waking vs. LCIR, and trends were sustained throughout the day. Analysis of 4-hour intervals showed foslevodopa and foscarbidopa patients had significant increases in good ON and reductions in OFF at week 12 compared to baseline that were sustained across the day (FIG. 27 and FIG. 28). foslevodopa and foscarbidopa patients had fewer daily motor-state transitions (means: 4.7 vs 7.2; p<0.001), and nearly twice as many foslevodopa and foscarbidopa patients had 56 transitions on average vs. LCIR (64.5% vs. 38.6%; p=0.007) (FIG. 29).

Conclusions: foslevodopa and foscarbidopa patients reached good ON faster after waking, had greater stability in OFF and good ON and had fewer motor-state fluctuations during the waking day compared to LCIR patients. Consistent motor-symptom control may improve patients' ability to perform daily activities and quality of life.

### Example 14: Improvement in Motor-State Stability Throughout the Day and Rapid Onset to Good ON Time in Patients with aPD Treated with Foslevodopa and Foscarbidopa Vs. LCIR

Title: Concomitant Medication Use and Levodopa Equivalent Daily Dose Requirements After Foslevodopa/foscarbidopa Initiation

Background: foscarbidopa/foslevodopa subcutaneous infusion is a soluble formulation of levodopa/carbidopa prodrugs delivered as continuous subcutaneous infusion (CSCI) 24-hour daily. foscarbidopa/foslevodopa subcutaneous infusion provides individually adjustable doses to control motor complications in patients with advanced PD (aPD).

Methods: Clinical Study B (Example 8) was a 52-week, phase 3, open-label, single-arm, multicenter, global (US, EU, Australia, and Japan) study to assess the local and systemic safety, tolerability, and efficacy of 24-hour daily CSCI of foscarbidopa/foslevodopa subcutaneous infusion. The foscarbidopa/foslevodopa subcutaneous infusion dose was individualized to achieve an optimal clinical response for each patient. Intake of prior and allowable concomitant medication classes (adamantane derivatives, tropine derivatives, catechol-O-methyltransferase [COMT] inhibitors, monoamine oxidase-B [MAO-B] inhibitors, tertiary amines, other anti-Parkinson drugs, and non-ergolinic dopamine agonists) were summarized by study visit. LEDD for LDP/CDP was evaluated at each study visit.

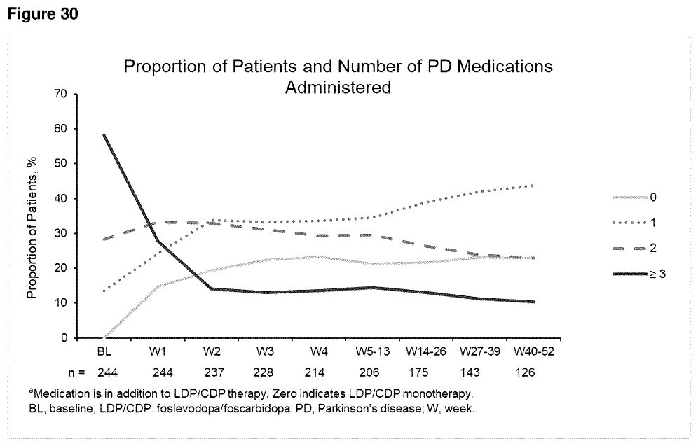

Results: The analysis included 244 patients with aPD. At baseline (BL), the proportion of patients taking 0, 1, 2, and ≥3 classes of PD medications were 0%, 13.5%, 28.3%, and 58.2%, respectively. At week 52, there was a reduction in the number of patients taking ≥3 PD medications (10.3%) that was associated with a shift in the number of patients taking 0, (LDP/CDP monotherapy; 23.0%), 1 (43.7%) and 2 (23.0%) classes of PD medications (FIG. 30). The LEDD of foscarbidopa/foslevodopa subcutaneous infusion (CSCI and extra doses) remained fairly stable over time (range, 1621.9-1847.0 mg/day).

Conclusions: As demonstrated by the treatment patterns observed, there was flexibility in the foscarbidopa/foslevodopa subcutaneous infusion dose and use of concomitant medications. For patients with aPD who initiated foscarbidopa/foslevodopa subcutaneous infusion treatment, 23% achieved monotherapy with LDP/CDP by week 52. The daily dose of foscarbidopa/foslevodopa subcutaneous infusion remained stable over time while the use of concomitant medications decreased, indicating a reduced need for co-medications to adequately control motor fluctuation in patients with aPD.

### REFERENCES

[1] L. V. Kalia, A. E. Lang, Parkinson's disease, Lancet 386(9996) (2015) 896-912.
[2] M. J. Armstrong, M. S. Okun, Diagnosis and Treatment of Parkinson Disease: A Review, Jama 323(6) (2020) 548-560.
[3] A. Antonini, K. R. Chaudhuri, P. Martinez-Martin, P. Odin, Oral and infusion levodopa-based strategies for managing motor complications in patients with Parkinson's disease, CNS drugs 24(2) (2010) 119-29.
[4] C. W. Olanow, K. Kieburtz, P. Odin, A. J. Espay, D. G. Standaert, H. H. Fernandez, A. Vanagunas, A. A. Othman, K. L. Widnell, W. Z. Robieson, Y. Pritchett, K. Chatamra, J. Benesh, R. A. Lenz, A. Antonini, L.H.S. Group, Continuous intrajejunal infusion of levodopa-carbidopa intestinal gel for patients with advanced Parkinson's disease: a randomised, controlled, double-blind, double-dummy study, Lancet Neurol 13(2) (2014) 141-9.
[5] M. Rosebraugh, E. A. Voight, E. M. Moussa, F. Jameel, X. Lou, G. G. Z. Zhang, P. T. Mayer, D. Stolarik, R. A. Carr, B. P. Enright, W. Liu, M. F. Facheris, P. R. Kym, foslevodopa/foscarbidopa: a new subcutaneous treatment for Parkinson's disease, Ann Neurol (2021).
[6] A. A. Othman, M. Rosebraugh, K. Chatamra, C. Locke, S. Dutta, Levodopa-carbidopa intestinal gel pharmacokinetics: Lower variability than oral levodopa-carbidopa, Journal of Parkinson's disease (2017).
[7] D. Nyholm, P. Odin, A. Johansson, K. Chatamra, C. Locke, S. Dutta, A. A. Othman, Pharmacokinetics of levodopa, carbidopa, and 3-O-methyldopa following 16-hour jejunal infusion of levodopa-carbidopa intestinal gel in advanced Parkinson's disease patients, The AAPS journal 15(2) (2013) 316-23.
[8] M. Rosebraugh, W. Liu, M. Neenan, M. F. Facheris, foslevodopa/Foscarbidopa Is Well Tolerated and Maintains Stable Levodopa and Carbidopa Exposure Following Subcutaneous Infusion, Journal of Parkinson's disease 11(4) (2021) 1695-1702.
[9] K. C. Yeh, T. F. August, D. F. Bush, K. C. Lasseter, D. G. Musson, S. Schwartz, M. E. Smith, D. C. Titus, Pharmacokinetics and bioavailability of Sinemet CR: a summary of human studies, Neurology 39(11 Suppl 2) (1989) 25-38.
[10] R. A. Hauser, A. L. Ellenbogen, L. V. Metman, A. Hsu, M. J. O'Connell, N. B. Modi, H. M. Yao, S. H. Kell, S. K. Gupta, Crossover comparison of IPX066 and a standard levodopa formulation in advanced Parkinson's disease, Mov Disord 26(12) (2011) 2246-52.
[11] J. G. Nutt, W. R. Woodward, J. P. Hammerstad, J. H. Carter, J. L. Anderson, The “on-off” phenomenon in Parkinson's disease. Relation to levodopa absorption and transport, The New England journal of medicine 310(8) (1984) 483-8.
[12] R. J. Hardie, S. L. Malcolm, A. J. Lees, G. M. Stern, J. G. Allen, The pharmacokinetics of intravenous and oral levodopa in patients with Parkinson's disease who exhibit on-off fluctuations, British journal of clinical pharmacology 22(4) (1986) 429-36.
[13] J. G. Nutt, W. R. Woodward, J. L. Anderson, The effect of carbidopa on the pharmacokinetics of intravenously administered levodopa: the mechanism of action in the treatment of parkinsonism, Ann Neurol 18(5) (1985) 537-43.
[14] Tomlinson C L, Stowe R, Patel S, et al. Systematic review of levodopa dose equivalency reporting in Parkinson's disease. Mov Disord. 2010; 25(15):2649-53.
[15] Ricciardi L, et al. 24-Hour infusion of levodopa/carbidopa intestinal gel for nocturnal akinesia in advanced Parkinson's disease. Mov Disord. 2016 April; 31(4): 597-8.
[16] Nyholm D, et al. Long-term 24-h levodopa/carbidopa gel infusion in Parkinson's disease. Parkinsonism Relat Disord. 2012 September; 18(8): 1000-1.
[17] Cruse B, et al. 24-hour levodopa-carbidopa intestinal gel may reduce troublesome dyskinesia in advanced Parkinson's disease. NPJ Parkinsons Dis. 2018 Nov. 20; 4:34.

### INCORPORATION BY REFERENCE

All publications and patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating advanced Parkinson's disease in a subject by subcutaneous administration of a composition comprising foslevodopa and foscarbidopa wherein the composition achieves an increase in “On” time without troublesome dyskinesia.

2. A method of treating advanced Parkinson's disease by subcutaneous administration of a composition comprising foslevodopa and foscarbidopa wherein the composition achieves a decrease in “Off” time without troublesome dyskinesia.

3. A method of treating advanced Parkinson's disease by subcutaneous administration of a composition comprising foslevodopa and foscarbidopa wherein the composition achieves a decrease in sleep symptoms as assessed by the Parkinson's Disease Sleep Scale-2 (PDSS-2) total score.

4-6. (canceled)

7. The method of claim 1, wherein the composition is administered in an amount effective for reducing parkinsonian symptoms by at least 46% from baseline.

8. The method of claim 1, wherein the composition is administered in an amount effective for reducing the baseline MDS-UPDRS total score by at least 9 units.

9. The method of claim 1, wherein the composition is administered in an amount effective for reducing the baseline PDQ-39 score by at least 6.9 units.

10. The method of claim 1, wherein the composition is administered in an amount effective for reducing the baseline PDSS-2 total score by at least 2 units.

11. A method of claim 1, wherein the treatment duration is at least 10 days with no incidence of developing skin nodules.

12. A method of reducing incidences of “off” time of parkinsonian symptoms in a subject compared with the subject receiving oral administration of tablets containing levodopa and carbidopa, according to claim 1.

13. The method of claim 12, wherein the incidences of “off” time of parkinsonian symptoms in a subject are reduced while increasing “on” time without troublesome dyskinesia.

14. A method of improving average daily normalized “Off” time as assessed by the PD diary in a subject compared with the subject receiving oral administration of tablets containing levodopa and carbidopa according to claim 1.

15. A method of improving Motor Aspects of Experiences of Daily Living as assessed by the MDS-UPDRS Part II score in a subject compared with the subject receiving oral administration of tablets containing levodopa and carbidopa, according to claim 1.

16. A method of reducing morning akinesia in a subject compared with the subject receiving oral administration of tablets containing levodopa and carbidopa, according to claim 1.

17. A method of improving health-related quality of life as assessed by the EQ-5D-5L summary index in a subject compared with the subject receiving oral administration of tablets containing levodopa and carbidopa, according to claim 1.

18. A method of improving median bradykinesia score (BK50) as assessed by the wearable motion sensor device in a subject compared with the subject receiving oral administration of tablets containing levodopa and carbidopa, according to claim 1.

19. A method of improving interquartile range of bradykinesia score (BK75-BK25) as assessed by the wearable motion sensor device in a subject compared with the subject receiving oral administration of tablets containing levodopa and carbidopa, according to claim 1.

20. A method of improving median dyskinesia score (DK50) as assessed by the PKG wearable device in a subject compared with the subject receiving oral administration of tablets containing levodopa and carbidopa, according to claim 1.

21. A method of improving of tremor and daytime somnolence as assessed by the PKG wearable device in a subject compared with the subject receiving oral administration of tablets containing levodopa and carbidopa, according to claim 1.

22. A method of improving MDS-UPDRS Part I score, Part III score, Part IV score, and total score of Parts I-III in a subject compared with the subject receiving oral administration of tablets containing levodopa and carbidopa, according to claim 1.

23. A method of improving “On” time with non-troublesome dyskinesia, and “On” time with troublesome dyskinesia as assessed by a PD Diary in a subject compared with the subject receiving oral administration of tablets containing levodopa and carbidopa, according to claim 1 to a patient in need of treatment for Parkinson's disease.

24. A method of improving average daily normalized “Off” time, “On” time without troublesome dyskinesia, “On” time without dyskinesia, “On” time with non-troublesome dyskinesia, and “On” time with troublesome dyskinesia as assessed by the PD Diary in a subject compared with the subject receiving oral administration of tablets containing levodopa and carbidopa, according to claim 1 to a patient in need of treatment for Parkinson's disease.

25. A method of improving average daily absolute “Off” time, “On” time without troublesome dyskinesia, “On” time without dyskinesia, “On” time with non-troublesome dyskinesia, “On” time with troublesome dyskinesia, and “Asleep” time as assessed by the PD Diary without normalizing in a subject compared with the subject receiving oral administration of tablets containing levodopa and carbidopa, according to claim 1.

26. A method of improving PDSS-2 domain scores in a subject compared with the subject receiving oral administration of tablets containing levodopa and carbidopa, according to claim 1.

27. A method of improving PDQ-39 domain scores in a subject compared with the subject receiving oral administration of tablets containing levodopa and carbidopa, according to claim 1.

28. A method of improving EQ-5D-5L visual analogue scale (VAS) score in a subject compared with the subject receiving oral administration of tablets containing levodopa and carbidopa, according to claim 1.

29. The method of claim 1, wherein the weight ration of foslevodopa to foscarbidopa is about 20:1.

30. The method of claim 1, wherein the composition comprises about 240 mg/mL of foslevodopa and about 12 mg/mL of foscarbidopa.

31. The method of claim 1, wherein the composition has a pH of between about 6.6 and about 8.1.

32. A method of improving “On” time with non-troublesome dyskinesia, and “Off” time with non-troublesome dyskinesia as assessed by a PD Diary in a subject compared with the subject receiving oral administration of tablets containing levodopa and carbidopa, according to claim 1 to a patient in need of treatment for Parkinson's disease, wherein the average “On” time is between 2.5-4.5 hours and the average “Off” time is between 2.5-4.5 hours.

33. The method of claim 32, wherein the average “On” time is about 3.6 hours.

34. The method of claim 32, wherein the average “Off” time is about 3.4 hours.

35. The method of claim 1, wherein the composition has an osmolality up to about 2700 mOsmol/kg.

36. The method of claim 1, wherein the composition has an osmolality between about 2200 and about 2500 mOsmol/kg.

37. A method of improving “On” time with non-troublesome dyskinesia as assessed by a PD Diary in a subject compared with the subject receiving oral administration of tablets containing levodopa and carbidopa, according to claim 1 to a patient in need of treatment for Parkinson's disease, wherein the average time to reach “On” time is between 10-60 minutes relative to the subject receiving oral administration of tablets containing levodopa and carbidopa.

38. The method of claim 37, wherein the average time to reach “On” time is from about 15 minutes to about 50 minutes.

39. The method of claim 37, wherein the time to reach “On” time is about 28.9 minutes.

40. A method of improving “On” time with non-troublesome dyskinesia as assessed by a PD Diary in a subject compared with the subject receiving oral administration of tablets containing levodopa and carbidopa, according to claim 1 to a patient in need of treatment for Parkinson's disease, wherein the frequency of “On” time is about twice as frequent relative to the subject receiving oral administration of tablets containing levodopa and carbidopa.

41. A method of improving “Off” time with non-troublesome dyskinesia as assessed by a PD Diary in a subject compared with the subject receiving oral administration of tablets containing levodopa and carbidopa, according to claim 1 to a patient in need of treatment for Parkinson's disease, wherein the frequency of “Off” time within 30 minutes of waking is about one-fourth less frequent relative to the subject receiving oral administration of tablets containing levodopa and carbidopa.

42. The method of claim 1, wherein the frequency of “Off” time is sustained for at least 24 hours.
